(19) United States
(12) Patent Application Publication
(10) Pub. No.: US 2023/0310013 A1
(43) Pub. Date: Oct. 5, 2023

(54) APPARATUS, SYSTEM, AND METHOD FOR PATIENT-SPECIFIC INSTRUMENTATION

(71) Applicant: Treace Medical Concepts, Inc., PONTE VEDRA, FL (US)

(72) Inventors: Adam PERLER, St. Petersburg, FL (US); James Q. SPITLER, Winter Garden, FL (US)

(21) Appl. No.: 18/129,151

(22) Filed: Mar. 31, 2023

Related U.S. Application Data
(60) Provisional application No. 63/326,249, filed on Mar. 31, 2022.
(60) Provisional application No. 63/387,080, filed on Dec. 12, 2022.

(51) Int. Cl. A61B 17/17; A61B 17/15

(52) U.S. Cl. CPC A61B 17/1775; A61B 17/152; A61B 2017/565

(57) ABSTRACT

An apparatus, system, and method is disclosed for correcting a condition present in a patient. The apparatus may include a bone positioner having: a bone attachment feature configured to couple the bone positioner to at least one of a first bone and a second bone; and a positioning member configured to position the second bone a patient-specific distance relative to the first bone for remediating a condition present in a patient's foot. The apparatus may also include a trajectory guide configured to guide one or more fasteners into one or more bones at a patient-specific trajectory.

### CROSS-REFERENCE TO RELATED APPLICATIONS

This application claims the benefit of U.S. Provisional Application No. 63/326,249, filed Mar. 31, 2022, which is hereby incorporated by reference in its entirety. This application also claims the benefit of U.S. Provisional Application No. 63/387,080, filed Dec. 12, 2022, which is hereby incorporated by reference in its entirety.

### TECHNICAL FIELD

The present disclosure relates to surgical devices, systems, instruments, and methods. More specifically, the present disclosure relates to patient-specific instruments, implants, instruments, and/or methods of designing and using the same.

### BACKGROUND

Various bone conditions may be corrected using surgical procedures, in which one or more tendons, ligaments, and/or bones may be cut, replaced, repositioned, reoriented, reattached, fixated and/or fused. These surgical procedures require the surgeon to properly locate, position, and/or orient one or more osteotomy cuts, fixation guides, fixators, bone tunnels, points of attachment for ends of grafts or soft tissue and the like. Determining and locating an optimal location and trajectory for one or more steps of the surgical procedures and/or securing instruments that can guide or assist in steps of the surgical procedures such as performing osteotomies, deploying fixation, and the like, can be challenging, given conventional techniques and instruments. What is needed is one or more instruments to facilitate locating, aligning, orienting, planning, preparing for, initiating, executing, and/or completing such surgical procedures. Existing solutions for guiding orthopedic surgical procedures are inadequate and error prone.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1A is a flowchart diagram depicting a method for remediating a condition, according to one embodiment.

FIG. 1B is a flowchart diagram depicting a method for remediating a condition, according to one embodiment.

FIG. 2A is a dorsal perspective view of bones of a foot.

FIG. 2B is a lateral perspective view of bones of a foot.

FIG. 2C is a medial perspective view of bones of a foot.

FIG. 2D is a dorsal perspective view of bones of a foot.

FIG. 3 is a flowchart diagram depicting a method for generating one or more patient-specific instruments, according to one embodiment.

FIG. 4 illustrates an exemplary system configured to generate one or more patient-specific instruments, according to one embodiment.

FIG. 5 illustrates an exemplary system configured to generate one or more patient-specific instruments, according to one embodiment.

FIG. 6 illustrates an exemplary system for remediating a condition present in a patient's foot, according to one embodiment.

FIG. 7 is a side perspective view of a resection guide according to one embodiment, shown secured to a bone.

FIGS. 8A-8D are a top perspective, bottom perspective, top, and bottom, views respectively of the resection guide of FIG. 7.

FIG. 9A is a side perspective view of a resection guide according to one embodiment, shown secured to a bone.

FIG. 9B is a perspective view of a resection guide according to one embodiment, that includes a handle.

FIGS. 10A-10D are a top perspective, bottom perspective, top, and bottom, views respectively of a resection guide, according to one embodiment.

FIG. 11 is a side perspective view of a rotation guide according to one embodiment, shown secured to a bone.

FIGS. 12A-12D are a top perspective, bottom perspective, top, and bottom, views respectively of a rotation guide, according to one embodiment.

FIGS. 12E-12H are a front, back, left side, and right side, views respectively of a rotation guide, according to one embodiment.

FIG. 13 is an exploded view of the rotation guide of FIG. 11.

FIG. 14 is a perspective medial view of foot bones with a positioner according to one embodiment.

FIG. 15A is a perspective view of a positioner according to one embodiment.

FIG. 15B is an exploded view of a positioner according to one embodiment.

FIGS. 16A-16H illustrate views of a positioner according to one or more embodiments.

FIG. 17 is a dorsal perspective view of a positioner according to one embodiment.

FIG. 18A is an anterior view of a positioner according to one embodiment.

FIG. 18B is an anterior view of a positioner according to one embodiment.

FIG. 19 is a side view of a positioner according to one embodiment.

FIG. 20 illustrates an exemplary system for remediating a condition present in a patient's foot, according to one embodiment.

FIG. 21 is a flowchart diagram depicting a method for remediating a condition, according to one embodiment.

FIGS. 22A-22D illustrate different views of one or more stages in a surgical procedure that includes one or more embodiments of the present disclosure.

FIGS. 23A-23E illustrate different views of one or more stages in a surgical procedure that includes one or more embodiments of the present disclosure.

FIGS. 24A-24C illustrate different views of one or more stages in a surgical procedure that includes one or more embodiments of the present disclosure.

FIGS. 25A-25C illustrate different views of one or more stages in a surgical procedure that includes one or more embodiments of the present disclosure.

FIGS. 26A-26D illustrate different views of one or more stages in a surgical procedure that includes one or more embodiments of the present disclosure.

FIG. 27 illustrates a stage in a surgical procedure that includes one or more embodiments of the present disclosure.

FIG. 28 illustrates a stage in a surgical procedure that includes one or more embodiments of the present disclosure.

FIG. 29 illustrates a stage in a surgical procedure that includes one or more embodiments of the present disclosure.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the disclosure but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. (Search “coupling” on Wikipedia.com Jul. 26, 2021. CC-BY-SA 3.0 Modified. Accessed Jul. 27, 2021.) A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

As used herein, a “marking” or “marker” refers to a symbol, letter, lettering, word, phrase, icon, design, color, diagram, indicator, figure, or combination of these designed, intended, structured, organized, configured, programmed, arranged, or engineered to communication information and/or a message to a user receiving, viewing, or encountering the marking. The marking can include one or more of a tactile signal, a visual signal or indication, an audible signal, and the like. In one embodiment, a marking may comprise a number or set letters, symbols, or words positioned on a surface, structure, color, color scheme, or device to convey a desired message or set of information.

“Patient specific” refers to a feature, an attribute, a characteristic, a structure, function, structure, device, guide, tool, instrument, apparatus, member, component, system, assembly, module, or subsystem or the like that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific attribute or feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, a trajectory for an instrument, implant, or anatomical part of a patient, a lateral offset, and/or other features.

“Patient-specific instrument” refers to an instrument, implant, or guide designed, engineered, and/or fabricated for use with a specific patient. In one aspect, a patient-specific instrument is unique to a patient and may include features unique to the patient such as a surface contour or other features.

“Patient-specific positioning guide” or “Patient-specific positioner” refers to an instrument, implant, positioner, structure, or guide designed, engineered, and/or fabricated for use as a positioner with a specific patient. In one aspect, a patient-specific positioning guide is unique to a patient and may include features unique to the patient such as patient-specific offsets, translation distances, openings, angles, orientations, anchor a surface contour or other features.

“Patient-specific cutting guide” refers to a cutting guide designed, engineered, and/or fabricated for use with a specific patient. In one aspect, a patient-specific cutting guide is unique to a patient and may include features unique to the patient such as a surface contour or other features.

“Patient-specific resection guide” refers to a guide designed, engineered, and/or fabricated for use in resection for a specific patient. In one aspect, a patient-specific resection guide is unique to a patient and may include features unique to the patient such as a surface contour or other features.

“Patient-specific trajectory guide” refers to a trajectory guide designed, engineered, and/or fabricated for use with a specific patient. In one aspect, a patient-specific trajectory guide is unique to a single patient and may include features unique to the patient such as a surface contour or other features.

“Patient specific instrument” (PSI) refers to a structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient. In certain aspects, one patient. In one aspect, a patient specific instrument is unique to a single patient and may include features unique to the patient such as a surface contour, component position, component orientation, and/or other features. In other aspects, one patient specific instrument may be useable with a number of patients having a particular class of characteristics.

As used herein, a “handle” or “knob” refers to a structure used to hold, control, or manipulate a device, apparatus, component, tool, or the like. A “handle” may be designed to be grasped and/or held using one or two hands of a user. In certain embodiments, a handle or knob may be an elongated structure. In one embodiment, a knob may be a shorter stubby structure.

As used herein, “implant” refers to a medical device manufactured to replace a missing biological structure, support a damaged biological structure, or enhance an existing biological structure. Often medical implants are man-made devices, but implants can also be natural occurring structures. The surface of implants that contact the body may be made of, or include a biomedical material such as titanium, cobalt chrome, stainless steel, carbon fiber, another metallic alloy, silicone, polymer, Synthetic polyvinyl alcohol (PVA) hydrogels, biomaterials, biocompatible polymers such as PolyEther Ether Ketone (PEEK) or a polylactide polymer (e.g. PLLA) and/or others, or apatite, or any combination of these depending on what is functional and/or economical. Implants can have a variety of configurations and can be wholly, partially, and/or include a number of components that are flexible, semiflexible, pliable, elastic, supple, semi-rigid, or rigid. In some cases implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants can be used to treat bone fractures, osteoarthritis, scoliosis, spinal stenosis, discomfort, and pain. Examples of orthopedic implants include, but are not limited to, a wide variety of pins, rods, screws, anchors, spacers, sutures, all-suture implants, ball all-suture implants, self-locking suture implants, cross-threaded suture implants, plates used to anchor fractured bones while the bones heal or fuse together, and the like. (Search “implant (medicine)” on Wikipedia.com May 26, 2021. CC-BY-SA 3.0 Modified. Accessed Jun. 30, 2021.)

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others. In one embodiment, a body may include a housing or frame or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “bone engagement surface” refers to a surface of an object, instrument, or apparatus, such as an implant that is oriented toward or faces one or more bones of a patient. In one aspect, the bone engagement surface may abut, touch, or contact a surface of a bone. In another aspect, the bone engagement surface or parts of the bone engagement surface may be close to, but not abut, touch, or contact a surface of the bone. In certain aspects, the bone engagement surface can be configured to engage with a surface of one or more bones. Such a bone engagement surface may include projections and recesses that correspond to and match projections and recesses of the one or more bone surfaces.

As used herein, a “deploy” or “deployment” refers to an act, action, process, system, method, means, or apparatus for inserting an implant or prosthesis into a part, body part, and/or patient. “Deploy” or “deployment” can also refer to an act, action, process, system, method, means, or apparatus for placing something into therapeutic use. A device, system, component, medication, drug, compound, or nutrient may be deployed by a human operator, a mechanical device, an automated system, a computer system or program, a robotic system, or the like.

“Joint” or “Articulation” refers to the connection made between bones in a human or animal body which link the skeletal system to form a functional whole. Joints may be biomechanically classified as a simple joint, a compound joint, or a complex joint. Joints may be classified anatomically into groups such as joints of hand, elbow joints, wrist joints, axillary joints, sternoclavicular joints, vertebral articulations, temporomandibular joints, sacroiliac joints, hip joints, knee joints, articulations of foot, and the like. (Search “joint” on Wikipedia.com Dec. 19, 2021. CC-BY-SA 3.0 Modified. Accessed Jan. 20, 2022.)

“Topographical” refers to the physical distribution of parts, structures, or features on the surface of, or within, an organ or other anatomical structure, or organism. (Search “define topographical” on google.com. Oxford Languages, Copyright 2022. Oxford University Press. Web., Modified. Accessed 15 Feb. 2022.)

“Landmark registration features” or “Landmark” refers to a structure configured to engage with a feature, aspect, attribute, or characteristic of a first object to orient and/or position a second object that includes the landmark registration feature with respect to the first object. A variety of structures can serve as a landmark registration feature. For example, a landmark registration feature may include a protrusion, a projection, a tuberosity, a cavity, a void, a divot, a tab, an extension, a hook, a curve, or the like. In the context of bones of a patient a landmark registration feature can include any protuberance, void, divot, concave section, sesamoid, bone spur or other feature on, or extending from, a bone of a patient.

“Bone attachment feature” refers to a structure, feature, component, aspect configured to securely connect, couple, attach, and/or engage a structure, component, object, or body with a bone and/or a bone fragment. Examples of a bone attachment feature, include, but are not limited to, a pin, K-wire, screw, or other fastener alone, or in combination with, a hole, passage, and/or opening.

As used herein, “patient-specific osteotomy procedure” refers to an osteotomy procedure that has been adjusted, tailored, modified, or configured to specifically address the needs or desires or a particular patient. In certain aspects, one patient-specific osteotomy procedure may be useable in connection with only one patient. In other aspects, one patient-specific osteotomy procedure may be useable with a number of patients having a particular class of characteristics.

As used herein, a “stop” refers to an apparatus, instrument, structure, member, device, component, system, or assembly structured, organized, configured, designed, arranged, or engineered to prevent, limit, impede, stop, or restrict motion or movement and/or operation of the another object, member, structure, component, part, apparatus, system, or assembly.

As used herein, a “fastener”, “fixation device”, or “fastener system” refers to any structure configured, designed, or engineered to join two structures. Fasteners may be made of a variety of materials including metal, plastic, composite materials, metal alloys, plastic composites, and the like. Examples of fasteners include, but are not limited to screws, rivets, bolts, nails, snaps, hook and loop, set screws, bone screws, nuts, posts, pins, thumb screws, and the like. Other examples of fasteners include, but are not limited to wires, Kirschner wires (K-wire), anchors, bone anchors, plates, bone plates, intramedullary nails or rods or pins, implants, sutures, soft sutures, soft anchors, tethers, interbody cages, fusion cages, and the like.

In certain embodiments, the term fastener may refer to a fastener system that includes two or more structures configured to combine to serve as a fastener. An example of a fastener system is a rod or shaft having external threads and an opening or bore within another structure having corresponding internal threads configured to engage the external threads of the rod or shaft.

In certain embodiments, the term fastener may be used with an adjective that identifies an object or structure that the fastener may be particularly configured, designed, or engineered to engage, connect to, join, contact, or couple together with one or more other structures of the same or different types. For example, a “bone fastener” may refer to an apparatus for joining or connecting one or more bones, one or more bone portions, soft tissue and a bone or bone portion, hard tissue and a bone or bone portion, an apparatus and a bone or portion of bone, or the like.

In certain embodiments, a fastener may be a temporary fastener. A temporary fastener is configured to engage and serve a fastening function for a relatively short period of time. Typically, a temporary fastener is configured to be used until another procedure or operation is completed and/or until a particular event. In certain embodiments, a user may remove or disengage a temporary fastener. Alternatively, or in addition, another structure, event, or machine may cause the temporary fastener to become disengaged.

As used herein, a “fixator” refers to an apparatus, instrument, structure, device, component, member, system, assembly, or module structured, organized, configured, designed, arranged, or engineered to connect two bones or bone fragments or a single bone or bone fragment and another fixator to position and retain the bone or bone fragments in a desired position and/or orientation. Examples of fixators include both those for external fixation as well as those for internal fixation and include, but are not limited to pins, wires, Kirschner wires, screws, anchors, bone anchors, plates, bone plates, intramedullary nails or rods or pins, implants, interbody cages, fusion cages, and the like.

As used herein, an “anchor” refers to an apparatus, instrument, structure, member, part, device, component, system, or assembly structured, organized, configured, designed, arranged, or engineered to secure, retain, stop, and/or hold, an object to or at a fixed point, position, or location. Often, an anchor is coupled and/or connected to a flexible member such as a tether, chain, rope, wire, thread, suture, suture tape, or other like object. Alternatively, or in addition, an anchor may also be coupled, connected, and/or joined to a rigid object or structure. In certain embodiments, an anchor can be a fixation device. Said another way, a fixation device can function as an anchor.

“Connector” refers to any structure configured, engineered, designed, adapted, and/or arranged to connect one structure, component, element, or apparatus to another structure, component, element, or apparatus. A connector can be rigid, pliable, elastic, flexible, and/or semiflexible. Examples of a connector include but are not limited any fastener.

As used herein, “manufacturing tool” or “fabrication tool” refers to a manufacturing or fabrication process, tool, system, or apparatus which creates an object, device, apparatus, feature, or component using one or more source materials. A manufacturing tool or fabrication tool can use a variety of manufacturing processes, including but not limited to additive manufacturing, subtractive manufacturing, forging, casting, and the like. The manufacturing tool can use a variety of materials including polymers, thermoplastics, metals, biocompatible materials, biodegradable materials, ceramics, biochemicals, and the like. A manufacturing tool may be operated manually by an operator, automatically using a computer numerical controller (CNC), or a combination of these techniques.

As used herein, “osteotomy procedure” or “surgical osteotomy” or “osteotomy” refers to a surgical operation in which one or more bones are cut to shorten or lengthen them or to change their alignment. The procedure can include removing one or more portions of bone and/or adding one or more portions of bone or bone substitutes. (Search “osteotomy” on Wikipedia.com Feb. 3, 22, 2021. CC-BY-SA 3.0 Modified. Accessed Feb. 15, 2022.) As used herein, “patient-specific osteotomy procedure” refers to an osteotomy procedure that has been adjusted, tailored, modified, or configured to specifically address the anatomy, physiology, condition, abnormalities, needs, or desires of a particular patient. In certain aspects, one patient-specific osteotomy procedure may be useable in connection with only one patient. In other aspects, one patient-specific osteotomy procedure may be useable with a number of patients having a particular class of characteristics. In certain aspects, a patient-specific osteotomy procedure may refer to a non-patient-specific osteotomy procedure that includes one or more patient-specific implants and/or instrumentation. In another aspects, a patient-specific osteotomy procedure may refer to a patient-specific osteotomy procedure that includes one or more patient-specific implants, patient-specific surgical steps, and/or patient-specific instrumentation.

“Wedge osteotomy” refers to an osteotomy procedure in which one or more wedges are used as part of the procedure. Generally, wedge osteotomies can be of one of two types, open wedge and closing wedge. The type of osteotomy refers to how the procedure changes the relation between two parts of a bone involved in the osteotomy. In an open wedge osteotomy a wedge of bone or graft or other material is inserted in between two parts of a bone. Consequently, a wedge shape is “opened” in the bone. In a close wedge osteotomy or closing wedge osteotomy a wedge of bone is removed from a bone. Consequently, a wedge shape formed in the bone is “closed.”

As used herein, “anatomic data” refers to data identified, used, collected, gathered, and/or generated in connection with an anatomy of a human or animal. Examples of anatomic data may include location data for structures, both independent, and those connected to other structures within a coordinate system. Anatomic data may also include data that labels or identifies one or more anatomical structures. Anatomic data can include volumetric data, material composition data, and/or the like. Anatomic data can be generated based on medical imaging data or measurements using a variety of instruments including monitors and/or sensors. Anatomic data can be gathered, measured, or collected from anatomical models and/or can be used to generate, manipulate, or modify anatomical models.

A bone model or anatomic model of a patient's body or body part(s) may be generated by computing devices that analyze medical imaging images. Structures of a patient's body can be determined using a process called segmentation.

“Positioner” or “positioning guide” refers to any structure, apparatus, surface, device, system, feature, or aspect configured to position, move, translate, manipulate, or arrange one object in relation to another. In certain embodiments, a positioner can be used for one step in surgical procedure to position, arrange, orient, and/or reduce one bone or bone fragment relative to another. In such embodiments, the positioner may be referred to as a bone positioner. In certain embodiments, the term positioner or positioning guide may be preceded by an adjective that identifies the structure, implement, component, or instrument that may be used with, positioned by, and/or guided by with the positioner. For example, a “pin positioner” may be configured to accept a pin or wire such as a K-wire and serve to position or place the pin relative to another structure such as a bone.

“Reduction guide” or “reducer” refers to any structure, apparatus, surface, device, system, feature, or aspect configured, designed, engineered, or fabricated to reduce or aide a user in the reduction of one bone or bone fragment or implant in relation to another bone or bone fragment or implant.

“Rotation guide” or “rotator” refers to any structure, apparatus, surface, device, system, feature, or aspect configured, designed, engineered, or fabricated to rotate or aide a user in the rotation of one structure relative to another structure. In certain embodiments, a rotation guide or rotator may be used to help a surgeon rotate one or more bones, parts of bones, bone fragment, an implant, or other anatomical structure, either alone or in relation to another one or more bones, parts of bones, bone fragments, implants, or other anatomical structures.

“Trajectory guide” or “trajectory indicator” or “targeting guide” refers to any structure, apparatus, surface, device, system, feature, or aspect configured to indicate, identify, guide, place, position, or otherwise assist in marking or deploying a fastener or other structure along a desired trajectory for one or more subsequent steps in a procedure.

As used herein, a “guard” refers to an apparatus, instrument, structure, member, device, component, system, or assembly structured, organized, configured, designed, arranged, or engineered to prevent, limit, impede, stop, or restrict motion, action, or movement and/or operation of the another object, member, structure, component, part, apparatus, system, or assembly beyond a certain parameter such as a boundary. Said another way, a “guard” refers to an apparatus, instrument, structure, member, device, component, system, or assembly structured, organized, configured, designed, arranged, or engineered to retain, maintain, hold, keep, or restrict motion, action, or movement and/or operation of the another object, member, structure, component, part, apparatus, system, or assembly within or at one or more parameters such as a boundary.

As used herein, “artificial intelligence” refers to intelligence demonstrated by machines, unlike the natural intelligence displayed by humans and animals, which involves consciousness and emotionality. The distinction between artificial intelligence and natural intelligence categories is often revealed by the acronym chosen. ‘Strong’ AI is usually labelled as artificial general intelligence (AGI) while attempts to emulate ‘natural’ intelligence have been called artificial biological intelligence (ABI). Leading AI textbooks define the field as the study of “intelligent agents”: any device that perceives its environment and takes actions that maximize its chance of achieving its goals. The term “artificial intelligence” can also be used to describe machines that mimic “cognitive” functions that humans associate with the human mind, such as “learning” and “problem solving”. (Search “artificial intelligence” on Wikipedia.com Jun. 25, 2021. CC-BY-SA 3.0 Modified. Accessed Jun. 25, 2021.)

As used herein, “segmentation” or “image segmentation” refers the process of partitioning an image into different meaningful segments. These segments may correspond to different tissue classes, organs, pathologies, bones, or other biologically relevant structures. Medical image segmentation accommodates imaging ambiguities such as by low contrast, noise, and other imaging ambiguities.

Certain computer vision techniques can be used or adapted for image segmentation. For example, the techniques and or algorithms for segmentation may include, but are not limited to: Atlas-Based Segmentation: For many applications, a clinical expert can manually label several images; segmenting unseen images is a matter of extrapolating from these manually labeled training images. Methods of this style are typically referred to as atlas-based segmentation methods. Parametric atlas methods typically combine these training images into a single atlas image, while nonparametric atlas methods typically use all of the training images separately. Atlas-based methods usually require the use of image registration in order to align the atlas image or images to a new, unseen image.

Image registration is a process of correctly aligning images; Shape-Based Segmentation: Many methods parametrize a template shape for a given structure, often relying on control points along the boundary. The entire shape is then deformed to match a new image. Two of the most common shape-based techniques are Active Shape Models and Active Appearance Models; Image-Based Segmentation: Some methods initiate a template and refine its shape according to the image data while minimizing integral error measures, like the Active contour model and its variations; Interactive Segmentation: Interactive methods are useful when clinicians can provide some information, such as a seed region or rough outline of the region to segment. An algorithm can then iteratively refine such a segmentation, with or without guidance from the clinician. Manual segmentation, using tools such as a paint brush to explicitly define the tissue class of each pixel, remains the gold standard for many imaging applications. Recently, principles from feedback control theory have been incorporated into segmentation, which give the user much greater flexibility and allow for the automatic correction of errors; Subjective surface Segmentation: This method is based on the idea of evolution of segmentation function which is governed by an advection-diffusion model. To segment an object, a segmentation seed is needed (that is the starting point that determines the approximate position of the object in the image). Consequently, an initial segmentation function is constructed. With the subjective surface method, the position of the seed is the main factor determining the form of this segmentation function; and Hybrid segmentation which is based on combination of methods. (Search “medical image computing” on Wikipedia.com Jun. 24, 2021. CC-BY-SA 3.0 Modified. Accessed Jun. 24, 2021.)

As used herein, “medical imaging” refers to a technique and process of imaging the interior of a body for clinical analysis and medical intervention, as well as visual representation of the function of some organs or tissues (physiology). Medical imaging seeks to reveal internal structures hidden by the skin and bones, as well as to diagnose and treat disease. Medical imaging may be used to establish a database of normal anatomy and physiology to make possible identification of abnormalities. Medical imaging in its widest sense, is part of biological imaging and incorporates radiology, which uses the imaging technologies of X-ray radiography, magnetic resonance imaging, ultrasound, endoscopy, elastography, tactile imaging, thermography, medical photography, nuclear medicine functional imaging techniques as positron emission tomography (PET) and single-photon emission computed tomography (SPECT). Another form of X-ray radiography includes computerized tomography (CT) scans in which a computer controls the position of the X-ray sources and detectors. Magnetic Resonance Imaging (MRI) is another medical imaging technology. Measurement and recording techniques that are not primarily designed to produce images, such as electroencephalography (EEG), magnetoencephalography (MEG), electrocardiography (ECG), and others, represent other technologies that produce data susceptible to representation as a parameter graph vs. time or maps that contain data about the measurement locations. In certain embodiments bone imaging includes devices that scan and gather bone density anatomic data. These technologies may be considered forms of medical imaging in certain disciplines. (Search “medical imaging” on Wikipedia.com Jun. 16, 2021. CC-BY-SA 3.0 Modified. Accessed Jun. 23, 2021.) Data, including images, text, and other data associated with medical imaging is referred to as patient imaging data. As used herein, “patient imaging data” refers to data identified, used, collected, gathered, and/or generated in connection with medical imaging and/or medical imaging data. Patient imaging data can be shared between users, systems, patients, and professionals using a common data format referred to as Digital Imaging and Communications in Medicine (DICOM) data. DICOM data is a standard format for storing, viewing, retrieving, and sharing medical images.

As used herein, “medical image computing” or “medical image processing” refers to systems, software, hardware, components, and/or apparatus that involve and combine the fields of computer science, information engineering, electrical engineering, physics, mathematics and medicine. Medical image computing develops computational and mathematical methods for working with medical images and their use for biomedical research and clinical care. One goal for medical image computing is to extract clinically relevant information or knowledge from medical images. While closely related to the field of medical imaging, medical image computing focuses on the computational analysis of the images, not their acquisition. The methods can be grouped into several broad categories: image segmentation, image registration, image-based physiological modeling, and others. (Search “medical image computing” on Wikipedia.com Jun. 24, 2021. CC-BY-SA 3.0 Modified. Accessed Jun. 24, 2021.) Medical image computing may include one or more processors or controllers on one or more computing devices. Such processors or controllers may be referred to herein as medical image processors. Medical imaging and medical image computing together can provide systems and methods to image, quantify and fuse both structural and functional information about a patient in vivo. These two technologies include the transformation of computational models to represent specific subjects/patients, thus paving the way for personalized computational models. Individualization of generic computational models through imaging can be realized in three complementary directions: definition of the subject-specific computational domain (anatomy) and related subdomains (tissue types); definition of boundary and initial conditions from (dynamic and/or functional) imaging; and characterization of structural and functional tissue properties. Medical imaging and medical image computing enable in the translation of models to the clinical setting with both diagnostic and therapeutic applications. (Id.) In certain embodiments, medical image computing can be used to generate a bone model, a patient-specific model, and/or a patent specific instrument from medical imaging and/or medical imaging data.

As used herein, “model” refers to an informative representation of an object, person or system. Representational models can be broadly divided into the concrete (e.g. physical form) and the abstract (e.g. behavioral patterns, especially as expressed in mathematical form). In abstract form, certain models may be based on data used in a computer system or software program to represent the model. Such models can be referred to as computer models. Computer models can be used to display the model, modify the model, print the model (either on a 2D medium or using a 3D printer or additive manufacturing technology). Computer models can also be used in environments with models of other objects, people, or systems. Computer models can also be used to generate simulations, display in virtual environment systems, display in augmented reality systems, or the like. Computer models can be used in Computer Aided Design (CAD) and/or Computer Aided Manufacturing (CAM) systems. Certain models may be identified with an adjective that identifies the object, person, or system the model represents. For example, a “bone” model is a model of a bone, and a “heart” model is a model of a heart. (Search “model” on Wikipedia.com Jun. 13, 2021. CC-BY-SA 3.0 Modified. Accessed Jun. 23, 2021.) As used herein, “additive manufacturing” refers to a manufacturing process in which materials are joined together in a process that repeatedly builds one layer on top of another to generate a three-dimensional structure or object. Additive manufacturing may also be referred to using different terms including: additive processes, additive fabrication, additive techniques, additive layer manufacturing, layer manufacturing, freeform fabrication, ASTM F2792 (American Society for Testing and Materials), and 3D printing. Additive manufacturing can build the three-dimensional structure or object using computer-controlled equipment that applies successive layers of the material(s) based on a three-dimensional model that may be defined using Computer Aided Design (CAD) software. Additive manufacturing can use a variety of materials including polymers, thermoplastics, metals, ceramics, biochemicals, and the like. Additive manufacturing may provide unique benefits, as an implant together with the pores and/or lattices can be directly manufactured (without the need to generate molds, tool paths, perform any milling, and/or other manufacturing steps).

“Repository” refers to any data source or dataset that includes data or content. In one embodiment, a repository resides on a computing device. In another embodiment, a repository resides on a remote computing or remote storage device. A repository may comprise a file, a folder, a directory, a set of files, a set of folders, a set of directories, a database, an application, a software application, content of a text, content of an email, content of a calendar entry, and the like. A repository, in one embodiment, comprises unstructured data. A repository, in one embodiment, comprises structured data such as a table, an array, a queue, a look up table, a hash table, a heap, a stack, or the like. A repository may store data in any format including binary, text, encrypted, unencrypted, a proprietary format, or the like.

As used herein, a “sleeve” refers to structure that is narrow and longer longitudinally than the structure is wide. In certain embodiments, a sleeve serves to surround, enclose, wrap, and/or contain something else. In certain embodiments, a sleeve may surround, enclose, wrap, and/or contain a passage or void. (Search “sleeve” on wordhippo.com. WordHippo, 2021. Web. Accessed 15 Nov. 2021. Modified.) In certain embodiments, the term sleeve may be preceded by an adjective that identifies the structure, implement, component or instrument that may be used with, inserted into or associated with the sleeve. For example, a “pin sleeve” may be configured to accept a pin or wire such as a K-wire, a “drive sleeve” may be configured to accept a drill or drill bit, a “fixation member sleeve” may be configured to accept a fastener or fixation member.

As used herein, “registration” or “image registration” refers to a method, process, module, component, apparatus, and/or system that seeks to achieve precision in the alignment of two images. As used here, “image” may refer to either or both an image of a structure or object and another image or a model (e.g., a computer based model or a physical model, in either two dimensions or three dimensions). In the simplest case of image registration, two images are aligned. One image may serve as the target image and the other as a source image; the source image is transformed, positioned, realigned, and/or modified to match the target image. An optimization procedure may be applied that updates the transformation of the source image based on a similarity value that evaluates the current quality of the alignment. An iterative procedure of optimization may be repeated until a (local) optimum is found. An example is the registration of CT and PET images to combine structural and metabolic information. Image registration can be used in a variety of medical applications: Studying temporal changes; Longitudinal studies may acquire images over several months or years to study long-term processes, such as disease progression. Time series correspond to images acquired within the same session (seconds or minutes). Time series images can be used to study cognitive processes, heart deformations and respiration; Combining complementary information from different imaging modalities. One example may be the fusion of anatomical and functional information.

Since the size and shape of structures vary across modalities, evaluating the alignment quality can be more challenging. Thus, similarity measures such as mutual information may be used; Characterizing a population of subjects. In contrast to intra-subject registration, a one-to-one mapping may not exist between subjects, depending on the structural variability of the organ of interest. Inter-subject registration may be used for atlas construction in computational anatomy. Here, the objective may be to statistically model the anatomy of organs across subjects; Computer-assisted surgery: in computer-assisted surgery pre-operative images such as CT or MRI may be registered to intra-operative images or tracking systems to facilitate image guidance or navigation. There may be several considerations made when performing image registration: The transformation model. Common choices are rigid, affine, and deformable transformation models. B-spline and thin plate spline models are commonly used for parameterized transformation fields. Non-parametric or dense deformation fields carry a displacement vector at every grid location; this may use additional regularization constraints. A specific class of deformation fields are diffeomorphisms, which are invertible transformations with a smooth inverse; The similarity metric. A distance or similarity function is used to quantify the registration quality. This similarity can be calculated either on the original images or on features extracted from the images. Common similarity measures are sum of squared distances (SSD), correlation coefficient, and mutual information. The choice of similarity measure depends on whether the images are from the same modality; the acquisition noise can also play a role in this decision. For example, SSD may be the optimal similarity measure for images of the same modality with Gaussian noise. However, the image statistics in ultrasound may be significantly different from Gaussian noise, leading to the introduction of ultrasound specific similarity measures.

Multi-modal registration may use a more sophisticated similarity measure; alternatively, a different image representation can be used, such as structural representations or registering adjacent anatomy; The optimization procedure. Either continuous or discrete optimization is performed. For continuous optimization, gradient-based optimization techniques are applied to improve the convergence speed. (Search “medical image computing” on Wikipedia.com Jun. 24, 2021. CC-BY-SA 3.0 Modified. Accessed Jun. 25, 2021.)

As used herein, a “resection” refers to a method, procedure, or step that removes tissue from another anatomical structure or body. A resection is typically performed by a surgeon on a part of a body of a patient. A resection is one type of osteotomy. (Search “surgery” on Wikipedia.com May 26, 2021. CC-BY-SA 3.0 Modified. Accessed May 26, 2021.) Resection may be used as a noun or a verb. In the verb form, the term is “resect” and refers to an act of performing, or doing, a resection. Past tense of the verb resect is resected.

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region. “Malrotation” refers to a condition in which a part, typically a part of a patient's body has rotated from a normal position to an unnormal or uncommon position.

As used herein, a “guide” refers to a part, component, member, or structure designed, adapted, configured, or engineered to guide or direct one or more other parts, components, or structures. A guide may be part of, integrated with, connected to, attachable to, or coupled to, another structure, device, or instrument. In one embodiment, a guide may include a modifier that identifies a particular function, location, orientation, operation, type, and/or a particular structure of the guide. Examples of such modifiers applied to a guide, include, but are not limited to, “pin guide” that guides or directs one or more pins, a “cutting guide” that guides or directs the making or one or more cuts, a placement, deployment, or insertion guide that guides or directs the placement, positioning, orientation, deployment, installation, or insertion of a fastener and/or implant, a “cross fixation guide” that guides deployment of a fastener or fixation member, an “alignment guide” that guides the alignment of two or more objects or structures, a “resection guide” that serves to guide resection of soft or hard tissue, such as in an osteotomy, a “reduction guide” can serve to guide reduction of one or more bone segments or fragments, an “placement guide” that serves to identify how an object can be placed in relation to another object or structure, and the like. Furthermore, guides may include modifiers applied due to the procedure or location within a patient for which the guide is to be used. For example, where a guide is used at a joint, the guide may be referred to herein as an “arthrodesis guide”.

As used herein, “feature” refers to a distinctive attribute or aspect of something. (Search “feature” on google.com. Oxford Languages, 2021. Web. 20 Apr. 2021.) A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “attachment feature,” “securing feature,” “placement feature,” “protruding feature,” “engagement feature,” “disengagement feature,” “resection feature”, “guide feature”, and the like.

Those of skill in the art will appreciate that a resection feature may take a variety of forms and may include a single feature or one or more features that together form the resection feature. In certain embodiments, the resection feature may take the form of one or more slots or cut channels. Alternatively, or in addition, a resection feature may be referenced using other names including, but not limited to, channel, cut channels, and the like.

“Cut channel” refers to a channel, slot, hole, or opening, configured to facilitate making a cut. In certain embodiments, a cut channel is one example of a resection feature, resection member, and/or resection guide. “Rotation slot” refers to a channel, slot, hole, or opening, configured to facilitate rotating one structure in relation to another structure.

“Hole” refers to a gap, an opening, an aperture, a port, a portal, a space or recess in a structure, a void in a structure, or the like. In certain embodiments, a hole can refer to a structure configured specifically for receiving something and/or for allowing access. In certain embodiments, a hole can pass through a structure. In other embodiments, an opening can exist within a structure but not pass through the structure. A hole can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “hole” can include one or more modifiers that define specific types of “holes” based on the purpose, function, operation, position, or location of the “hole.” As one example, a “fastener hole” refers to an “hole” adapted, configured, designed, or engineered to accept or accommodate a “fastener.”

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance, but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening.” As one example, a “fastener opening” refers to an “opening” adapted, configured, designed, or engineered to accept or accommodate a “fastener.”

As used herein, an “interface,” “user interface,” or “engagement interface” refers to an area, a boundary, or a place at which two separate and/or independent structures, members, apparatus, assemblies, components, and/or systems join, connect, are coupled, or meet and act on, or communicate, mechanically and/or electronically, with each other. In certain embodiments, “interface” may refer to a surface forming a common boundary of two bodies, spaces, structures, members, apparatus, assemblies, components, or phases. (search “interface” on Merriam-Webster.com. Merriam-Webster, 2021. Web. 15 Nov. 2021. Modified.) In certain embodiments, the term interface may be used with an adjective that identifies a type or function for the interface. For example, an engagement or coupling interface may refer to one or more structures that interact, connect, or couple to mechanically join or connect two separate structures, each connected to a side of the interface. In another example, a user interface may refer to one or more mechanical, electrical, or electromechanical structures that interact with or enable a user to provide user input, instructions, input signals, data, or data values and receive output, output data, or feedback.

“Cortical bone” refers to a type of bone tissue. Cortical bone is a type of bone tissue typically found between an external surface of a bone and an interior area of the bone. Cortical bone is more dense and typically stronger structurally than other types of bone tissue. “Cortical surface” refers to a surface of cortical bone.

“Cortex” refers to an area of bone that extends from an external surface of the bone towards a center part of the bone. The cortex is typically comprised of cortical bone.

“Metatarsal” is a bone of a foot of a human or animal. In a human, a foot includes five metatarsals which are identified by number starting from the most medial metatarsal, which is referred to as a first metatarsal and moving laterally the next metatarsal is the second metatarsal, and the naming continues in like manner for the third, fourth, and fifth metatarsal. The metatarsal bone includes three parts a base is a part that is at a proximal end of the metatarsal, a head is a part that is at a distal end of the metatarsal, and a shaft connects the base to the head.

“Capital fragment” refers to a distal end of a metatarsal or other long bone has been separated from the metatarsal or other long bone by an osteotomy. Typically, the capital fragment includes at least a portion of, or all of a head of the metatarsal or other long bone.

“Transosseous placement feature” refers to a placement feature that extends through one or more bones and that enables, or facilitates, placement of another device, apparatus, or instrument.

“Patient specific feature” refers to a feature, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features. “Medial resection guide” refers to a resection guide designed, engineered, fabricated, or intended for use with, one, in, or about a medial part, section, surface, portion, or aspect of an anatomical structure such as a bone, digit, limb, or other anatomical structure for one or more steps of a resection procedure. “Lateral resection guide” refers to a resection guide designed, engineered, fabricated, or intended for use with, one, in, or about a lateral part, section, surface, portion, or aspect of an anatomical structure such as a bone, digit, limb, or other anatomical structure for one or more steps of a resection procedure.

“Bone fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

“Cut surface” refers to a surface of an object that is created or formed by the removal of one or more parts of the object that includes the original surface. Cut surfaces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface can include manual, mechanical, motorized, hydraulic, automated, robotic, and the like. In certain embodiments, the cut surface(s) are planar.

“Orientation” refers to a direction, angle, position, condition, state, or configuration of a first object, component, part, apparatus, system, or assembly relative to another object, component, part, apparatus, system, assembly, reference point, reference axis, or reference plane.

“Longitudinal axis” or “Long axis” refers to an axis of a structure, device, object, apparatus, or part thereof that extends from one end of a longest dimension to an opposite end. Typically, a longitudinal axis passes through a center of the structure, device, object, apparatus, or part thereof along the longitudinal axis. The center point used for the longitudinal axis may be a geometric center point and/or a mass center point.

As used herein, a “base” refers to a main or central structure, component, or part of a structure. A base is often a structure, component, or part upon which, or from which other structures extend into, out of, away from, are coupled to, or connect to. A base may have a variety of geometric shapes and configurations. A base may be rigid or pliable. A base may be solid or hollow. A base can have any number of sides. In one embodiment, a base may include a housing, frame, or framework for a larger system, component, structure, or device. In certain embodiments, a base can be a part at the bottom or underneath a structure designed to extend vertically when the structure is in a desired configuration or position.

“Cutting tool” refers to any tool that can be used to cut or resect another object. In particular, a cutting tool can refer to a manual or power tool for cutting or resecting tissue of a patient. Examples, of cutting tools include, but are not limited to, a burr, an oscillating saw, a reciprocating saw, a grater saw, a drill, a mill, a side-cutting burr, or the like.

The present disclosure discloses surgical systems and methods by which a bone condition, that can include a deformity, may be corrected or otherwise addressed. Known methods of addressing bone conditions are often limited to a finite range of discretely sized instruments. A patient with an unusual condition, or anatomy that falls between instrument sizes, may not be readily treated with such systems.

Furthermore, patient-specific instruments may be used for various other procedures on the foot, or on other bones of the musculoskeletal system. For example, patient-specific instruments and/or other instruments may be used for various procedures including resection and translation of a head of a long bone, determining where to perform an osteotomy on one or more joints or part of one or more bones, determining ligament or tendon attachment or anchoring points, determining where to form bone tunnels or position anchors, tendon or graft deployment, and the like.

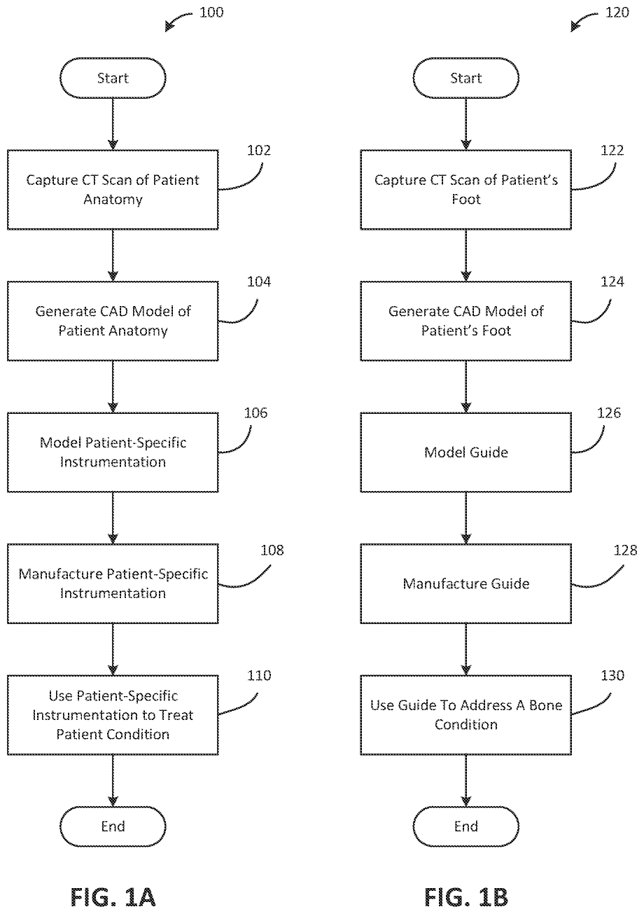

FIG. 1A is a flowchart diagram depicting a method 100 for correcting a bone condition, according to one embodiment. The method 100 may be used for any of a wide variety of bone conditions, including but not limited to deformities, fractures, joint failure, and/or the like. Further, the method 100 may provide correction with a wide variety of treatments, including but not limited to arthroplasty, arthrodesis, fracture repair, and/or the like.

As shown, the method 100 may begin with a step 102 in which a CT scan (or another three-dimensional image, also referred to as medical imaging) of the patient's anatomy is obtained. The step 102 may include capturing a scan of only the particular bone(s) to be treated, or may include capture of additional anatomic information, such as the surrounding tissues. Additionally or alternatively, the step 102 may include receiving a previously captured image, for example, at a design and/or fabrication facility. Performance of the step 102 may result in possession of a three-dimensional model of the patient's anatomy, or three-dimensional surface points that can be used to construct such a three-dimensional model.

After the step 102 has been carried out, the method 100 may proceed to a step 104 in which a CAD model of the patient's anatomy (including one or more bones) is generated. The CAD model may be one example of a bone model. The CAD model may be of any known format, including but not limited to SolidWorks, Catia, AutoCAD, or DXF. In some embodiments, customized software may be used to generate the CAD model from the CT scan. The CAD model may only include the bone(s) to be treated and/or may include surrounding tissues. In alternative embodiments, the step 104 may be omitted, as the CT scan may capture data that can directly be used in future steps without the need for conversion.

In one embodiment, the CAD model generated and/or patient-specific instrumentation, implants, and/or plan for conducting an operative procedure, may be enhanced by the use of advanced computer analysis system, machine learning, and/or automated/artificial intelligence. For example, these technologies may be used to revise a set of steps for a procedure such that a more desirable outcome is achieved.

In a step 106, the CAD model and/or CT scan data may be used to model patient-specific instrumentation that can be used to correct the condition, as it exists in the patient's anatomy. In some embodiments, any known CAD program may be used to view and/or manipulate the CAD model and/or CT scan, and generate one or more instruments that are matched specifically to the size and/or shape of the patient's bone(s). In some embodiments, such instrumentation may include a targeting guide, trajectory guide, drill guide, cutting guide, tendon trajectory guide, capital fragment positioning guide, or similar guide that can be attached to one or more bones, with one or more features that facilitate work on the one or more bones pursuant to a procedure such as arthroplasty or arthrodesis. In some embodiments, performance of the step 106 may include modelling an instrument with a bone engagement surface that is shaped to match the contour of a surface of the bone, such that the bone engagement surface can lie directly on the corresponding contour.

In a step 108, the model(s) may be used to manufacture patient-specific instrumentation and/or implants. This may be done via any known manufacturing method, including casting, forging, milling, additive manufacturing, and/or the like. Additive manufacturing may provide unique benefits, as the model may be directly used to manufacture the instrumentation and/or implants (without the need to generate molds, tool paths, and/or the like beforehand). Such instrumentation may optionally include a targeting guide, trajectory guide, drill guide, cutting guide, positioner, positioning guide, tendon trajectory guide, or the like.

In addition to, or in the alternative to the step 108, the model(s) may be used to select from available sizes of implants and/or instruments or instruments having various attributes and advise the surgeon accordingly. For example, where a range of guides are available for a given procedure, analysis of the CAD data may facilitate pre-operative selection of the optimal guide and/or optimal placement of the guide on the bone. Similarly, if a range of implants and/or instruments may be used for a given procedure, analysis of the CAD data may facilitate pre-operative selection of the optimal implant(s). More particularly, properly-sized spacers, screws, bone plates, and/or other hardware may be pre-operatively selected.

Thus, the result of the step 108 may provision, to the surgeon, of one or more of the following: (1) one or more patient-specific instruments; (2) one or more patient-specific implants; (3) an instrument, selected from one or more available instrument sizes and/or configurations; (4) an implant, selected from one or more available implant sizes and/or configurations; (5) instructions for which instrument(s) to select from available instrument sizes and/or configurations; (6) instructions for which implant(s) to select from available implant sizes and/or configurations; (7) instructions for proper positioning or anchorage of one or more instruments to be used in the procedure; and (8) instructions for proper positioning or anchorage of one or more implants to be used in the procedure. These items may be provided to the surgeon directly, or to a medical device company or representative, for subsequent delivery to the surgeon.

In a step 110, the manufactured instrumentation may be used in surgery to facilitate treatment of the condition. In some embodiments, this may include placing the modelled bone engagement surface against the corresponding contour of the bone used to obtain its shape, and then using the resection feature(s) to guide resection of one or more bones. Then the bone(s) may be further treated, for example, by attaching one or more joint replacement implants (in the case of joint arthroplasty), or by attaching bone segments together (in the case of arthrodesis or fracture repair). Prior to completion of the step 110, the instrumentation may be removed from the patient, and the surgical wound may be closed.

As mentioned previously, the method 100 may be used to correct a wide variety of bone conditions. One example of the method 100 will be shown and described in connection with FIG. 1B, for correction of a bunion deformity of the foot.

In certain embodiments, one or more of a method, apparatus, and/or system of the disclosed solution can be used for training a surgeon to perform a patient-specific procedure or technique. In one embodiment, the CAD model generated and/or patient-specific instrumentation, implants, and/or plan for conducting an operative procedure can be used to train a surgeon to perform a patient-specific procedure or technique.

In one example embodiment, a surgeon may submit a CT scan of a patient's foot to an apparatus or system that implements the disclosed solution. Next, a manual or automated process may be used to generate a CAD model and for making the measurements and correction desired for the patient. In the automated process, advanced computer analysis system, machine learning and automated/artificial intelligence may be used to generate a CAD model and/or one or more patient-specific instruments and/or operation plans. For example, a patient-specific instrument may be fabricated that is registered to the patient's anatomy using a computer-aided machine (CAM) tool. In addition, a CAM tool may be used to fabricate a 3D structure representative of the patient's anatomy, referred to herein as a patient-specific synthetic cadaver. (e.g. one or more bones of a patient's foot). Next, the patient-specific instrument and the patient-specific synthetic cadaver can be provided to a surgeon who can then rehearse an operation procedure in part or in full before going into an operating room with the patient.

In certain embodiments, the patient-specific instrument or instrument can be used to preposition and/or facilitate pre-drilling holes for a plate system for fixation purposes. Such plate systems may be optimally placed, per a CT scan, after a correction procedure for optimal fixation outcome. In another embodiment, the CAD model and/or automated process such as advanced computer analysis, machine learning and automated/artificial intelligence may be used to measure a depth of the a through a patient-specific resection guide for use with robotics apparatus and/or systems which would control the depth of each cut within the guide to protect vital structures below or adjacent to a bone being cut. In another embodiment, the CAD model and/or automated process such as advanced computer analysis, machine learning and automated/artificial intelligence may be used to define desired fastener (e.g. bone screw) length and/or trajectories through a patient-specific instrument and/or implant. The details for such lengths, trajectories, and components can be detailed in a report provided to the surgeon preparing to perform a procedure.

FIG. 1B is a flowchart diagram depicting a method 120 for correcting or remediating a bone condition, according to one embodiment. The method 120 may be used to prepare for an orthopedic procedure which corrects or remediates a bone, muscle, and/or tendon condition of a patient.

As shown, the method 120 may begin with a step 122 in which a CT scan (or another three-dimensional image) of the patient's foot is obtained. The step 122 may include capturing a scan of select bones of a patient or may include capturing additional anatomic information, such as the entire foot. Additionally or alternatively, the step 122 may include receipt of previously captured image data. Capture of the entire foot in the step 122 may facilitate proper alignment of the first metatarsal with the rest of the foot (for example, with the second metatarsal). Performance of the step 122 may result in generation of a three-dimensional model of the patient's foot, or three-dimensional surface points that can be used to construct such a three-dimensional model.

After the step 122 has been carried out, the method 120 may proceed to a step 124 in which a CAD model of the relevant portion of the patient's anatomy is generated. The CAD model may optionally include the bones of the entire foot, like the CT scan obtained in the step 122. In alternative embodiments, the step 124 may be omitted in favor of direct utilization of the CT scan data, as described in connection with the step 104.

In a step 126, the CAD model and/or CT scan data may be used to model patient-specific instrumentation that can be used to correct or remediate a bone condition. Such instrumentation may include a guide. In one example, the guide can seat or abut or contact a surface of a bone and including an opening that guides a trajectory for a fastener for a procedure. In some embodiments, performance of the step 126 may include modelling the guide with a bone engagement surface that is shaped to match contours of the surfaces of the bone, such that the bone engagement surface can lie directly on the corresponding contours of the bone.

In a step 128, the model(s) may be used to manufacture patient-specific instrumentation and/or instruments. This may include manufacturing an instrument with the bone engagement surface and/or other features as described above. As in the step 108, the step 128 may additionally or alternatively involve provision of one or more instruments and/or implants from among a plurality of predetermined configurations or sizes. Further, the step 128 may additionally, or alternatively, involve provision of instructions for placement and/or anchorage of one or more instruments and/or instruments to carry out the procedure.

In a step 130, the manufactured instrument may be used in surgery to facilitate treatment of the condition. In certain embodiments, a bone engagement surface of the instrument may be placed against the corresponding contours of the bone. The instrument may include an opening and/or trajectory guide to guide insertion of a trajectory guide such as a temporary fastener such as a K-wire. The instrument may then be removed, and the remaining steps of a surgical procedure performed.

Method 100 and method 120 are merely exemplary. Those of skill in the art will recognize that various steps of the method 100 and the method 120 may be reordered, omitted, and/or supplemented with additional steps not specifically shown or described herein.

As mentioned previously, the method 120 is one species of the method 100; the present disclosure encompasses many different procedures, performed with respect to many different bones and/or joints of the body. Exemplary steps and instrumentation for the method 120 will further be shown and described in connection with the present disclosure. Those of skill in the art will recognize that the method 120 may be used in connection with different instruments; likewise, the instruments of the present disclosure may be used in connection with methods different from the method 100 and the method 120.

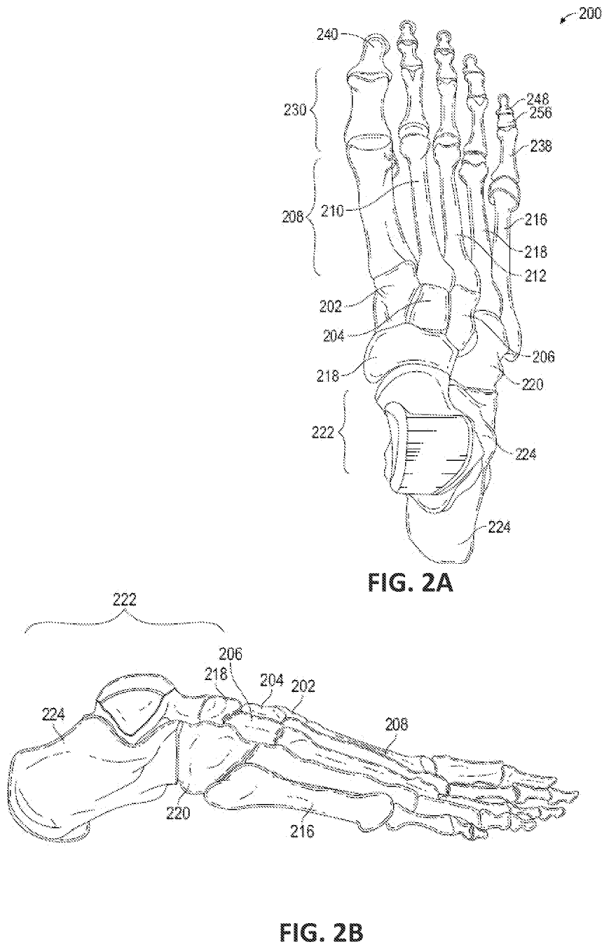

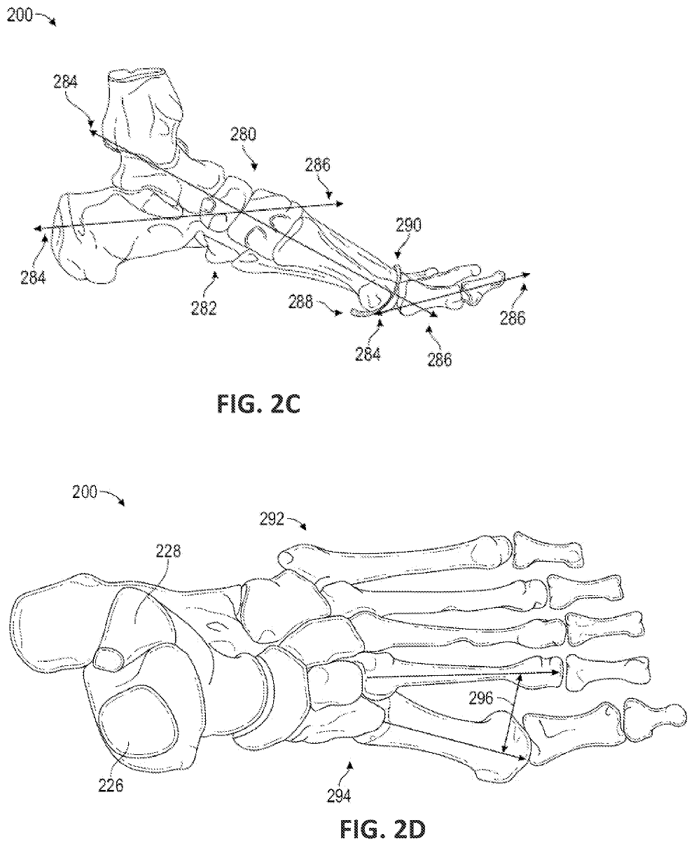

FIG. 2A is a perspective dorsal view of a foot 200. The foot 200 may have a medial cuneiform 202, an intermediate cuneiform 204, lateral cuneiform 206, a first metatarsal 208, a second metatarsal 210, third metatarsal 212, fourth metatarsal 214, fifth metatarsal 216, navicular 218, cuboid 220, talus 222, and calcaneus 224, among others. The medial cuneiform 202 and the intermediate cuneiform 204 may be joined together at a first metatarsocuneiform joint, and the first metatarsal 208 and the second metatarsal 210 may be joined together at a second metatarsocuneiform joint. The foot 200 includes a set of proximal phalanges numbered first through fifth (230, 232, 234, 236, 238) and a set of distal phalanges numbered first through fifth (240, 242, 244, 246, 248) and a set of middle phalanges numbered second through fifth (250, 252, 254, 256).

FIG. 2B is a perspective lateral view of a foot 200, with bones of the foot labeled.

FIG. 2C is a perspective medial view of a foot illustrating a dorsal side 280 and a plantar side 282. The foot 200, as illustrated, may have a tibia 226 and a fibula 228, among others. Dorsal refers to the top of the foot. Plantar refers to the bottom of the foot. Proximal 284 is defined as “closer to the primary attachment point”. Distal 286 is defined as “further away from the attachment point”. Plantar-flex or plantarflexion 288 means movement toward the plantar side 282 of a foot or hand, toward the sole or palm. Dorsiflex or dorsiflexion 290 means movement toward the dorsal side 280 of a foot or hand, toward the top. FIG. 2D is a perspective dorsal view of the foot 200. A transverse plane is the plane that shows the top of the foot. A lateral side 292 means a side furthest away from the midline of a body, or away from a plane of bilateral symmetry of the body. A medial side 294 means a side closest to the midline of a body, or toward a plane of bilateral symmetry of the body. For a Lapidus procedure, the intermetatarsal (IM) angle 296 is the angle to be corrected to remove the hallux valgus (bunion) deformity.

Every patient and/or condition is different; accordingly, the degree of angular adjustment needed in each direction may be different for every patient. Use of a patient-specific instrument may help the surgeon obtain an optimal realignment, target, or position a bone tunnel, position one or more resections and/or fasteners and the like. Thus, providing patient-specific instruments, jigs, and/or instrumentation may provide unique benefits.

The present patient-specific instrumentation may be used to correct a wide variety of conditions. Such conditions include, but are not limited to, angular deformities of one bone in either the lower or upper extremities (for example, tibial deformities, calcaneal deformities, femoral deformities, and radial deformities). The present disclosure may also be used to treat an interface between two bones (for example, the ankle joint, metatarsal cuneiform joint, lisfranc's joint, complex Charcot deformity, wrist joint, knee joint, etc.). As one example, an angular deformity or segmental malalignment in the forefoot may be treated, such as is found at the metatarsal cuneiform level, the midfoot level such as the navicular cuneiform junction, hindfoot at the calcaneal cuboid or subtalar joint or at the ankle between the tibia and talar junction. Additionally, patient-specific instruments could be used in the proximal leg between two bone segments or in the upper extremity such as found at the wrist or metacarpal levels.

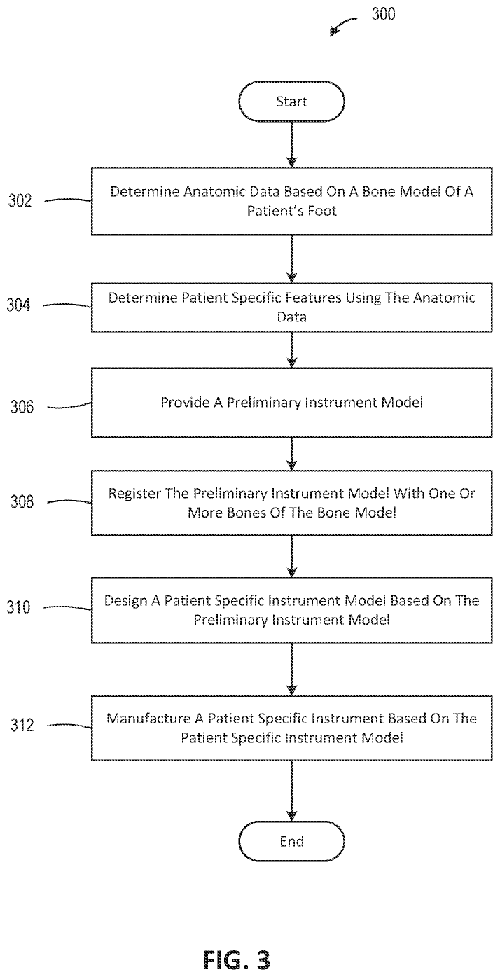

FIG. 3 illustrates a flowchart diagram depicting a method 300 for generating one or more instruments (which may or may not be patient-specific) configured to correct or address a bone or foot condition, according to one embodiment. Prior to steps of the method 300, a bone model (also referred to as CAD model above) is generated. The bone model may be generated using medical imaging of a patient's foot and may also be referred to as an anatomic model. The medical imaging image(s) may be used by computing devices to generate patient imaging data. The patient imaging data may be used to measure and account for orientation of one or more structures of a patient's anatomy. In certain embodiments, the patient imaging data may serve, or be a part of, anatomic data for a patient.

In one embodiment, the method 300 begins after a bone model of a patient's body or body part(s) is generated. In a first step 302, the method 300 may review the bone model and data associated with the bone model to determine anatomic data of a patient's foot.

After step 302, the method 300 may determine 304 one or more angles (e.g., trajectory angle) and/or patient-specific features for a procedure using the anatomic data. “Trajectory angle” refers to a recommended angle for deployment of an instrument, graft, body part, or resection feature angle relative to a bone of a patient for a procedure. In certain embodiments, determining steps, instruments, and/or implants for a corrective procedure may employ advanced computer analysis system, expert systems, machine learning, and/or automated/artificial intelligence.

Next, the method 300 may proceed and a preliminary instrument model is provided 306 from a repository of template models. A preliminary instrument model is a model of a preliminary instrument.

As used herein, “preliminary instrument” refers to a instrument configured, designed, and/or engineered to serve as a template, prototype, archetype, or starting point for creating, generating, or fabricating a patient-specific instrument. In one aspect, the preliminary instrument may be used, as-is, without any further changes, modifications, or adjustments and thus become a patient-specific instrument. In another aspect, the preliminary instrument may be modified, adjusted, or configured to more specifically address the goals, objectives, or needs of a patient or a surgeon and by way of the modifications become a patient-specific instrument. The patient-specific instrument can be used by a user, such as a surgeon, to guide steps in a surgical procedure, such as an osteotomy, graft harvest (e.g., autograft, allograft, or xenograft), minimally invasive surgical (MIS) procedure, and/or a tendon transfer procedure. Accordingly, a preliminary instrument model can be used to generate a patient-specific instrument. The patient-specific instrument model may be used in a surgical procedure to facilitate one or more steps of the procedure, and may be used to generate a patient-specific instrument that can be used in a surgical procedure for the patient.

In certain embodiments, the preliminary instrument model may be generated based on anatomic data and/or a bone model or a combination of these, and no model or predesigned structure, template, or prototype. Alternatively, or in addition, the preliminary instrument model may be, or may originate from, a template instrument model selected from a set of template instrument models. Each model in the set of template instrument models may be configured to fit for an average patient's foot. The template instrument model may subsequently be modified or revised by an automated process or manual process to generate the preliminary instrument model used in this disclosure.

As used herein, “template instrument” refers to an instrument configured, designed, and/or engineered to serve as a template for creating, generating, or fabricating a patient-specific instrument. In one aspect, the template instrument may be used, as-is, without any further changes, modifications, or adjustments and thus become a patient-specific instrument. In another aspect, the template instrument may be modified, adjusted, or configured to more specifically address the goals, objectives, or needs of a patient or a surgeon and by way of the modifications become a patient-specific instrument. The patient-specific instrument can be used by a user, such as a surgeon, to guide making one or more resections of a structure, such as a bone for a procedure. Accordingly, a template instrument model can be used to generate a patient-specific instrument model. The patient-specific instrument model may be used in a surgical procedure to address, correct, or mitigate effects of the identified deformity and may be used to generate a patient-specific instrument that can be used in a surgical procedure for the patient.

Next, the method 300 may register 308 the preliminary instrument model with one or more bones of the bone model. This step 308 facilitates customization and modification of the preliminary instrument model to generate a patient-specific instrument model from which a patient-specific instrument can be generated. The registration step 308 may combine two models and/or patient imaging data and positions both models for use in one system and/or in one model.

Next, the method 300 may design 310 a patient-specific instrument and/or procedure model based on the preliminary instrument model. The design step 310 may be completely automated or may optionally permit a user to make changes to a preliminary instrument model or partially completed patient-specific instrument model before the patient-specific instrument model is complete. A preliminary instrument model and patient-specific instrument model are two examples of an instrument model. As used herein, “instrument model” refers to a model, either physical or digital, that represents an instrument, tool, apparatus, or device. Examples, of an instrument model can include a cutting instrument model, a resection instrument model, an alignment instrument model, a reduction instrument model, a patient-specific tendon trajectory instrument model, graft harvesting instrument model, minimally invasive surgical (MIS) positioner model, or the like. In one embodiment, a patient-specific instrument and a patient-specific instrument model may be unique to a particular patient and that patient's anatomy and/or condition.

The method 300 may conclude by a step 312 in which patient-specific instrument may be manufactured based on the patient-specific instrument model. Various manufacturing tools, devices, systems, and/or techniques can be used to manufacture the patient-specific instrument.

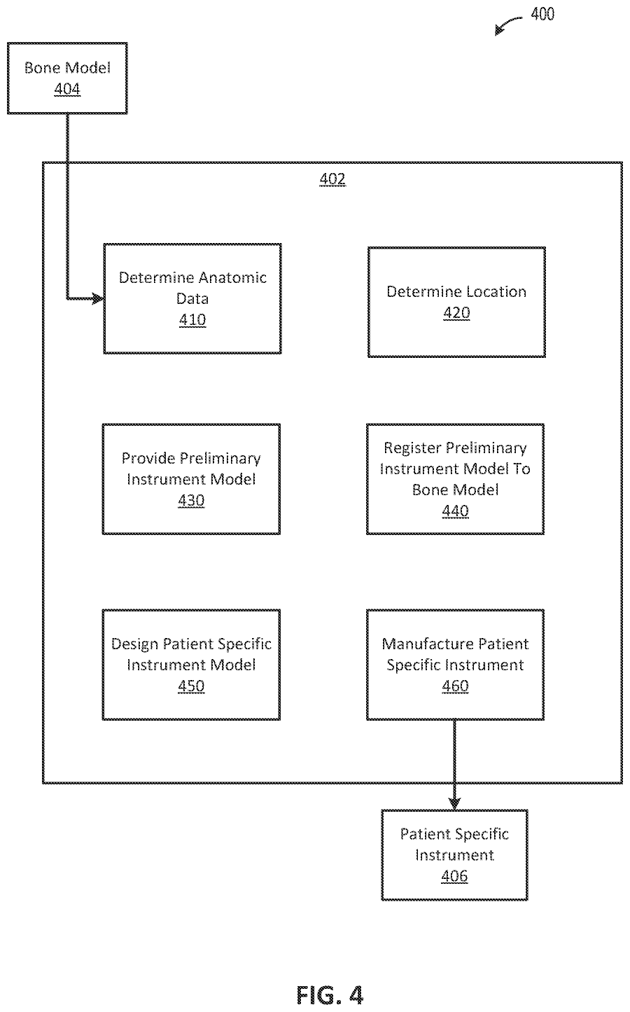

FIG. 4 illustrates an exemplary system 400 configured to generate one or more patient-specific instruments configured to facilitate surgical procedures, according to one embodiment. The system 400 may include an apparatus 402 configured to accept, review, receive or reference a bone model 404 and provide a patient-specific instrument 406. In one embodiment, the apparatus 402 is a computing device. In another embodiment, the apparatus 402 may be a combination of computing devices and/or software components or a single software component such as a software application.

The apparatus 402 may include a determination module 410, a location module 420, a provision module 430, a registration module 440, a design module 450, and a manufacturing module 460. Each of which may be implemented in one or more of software, hardware, or a combination of hardware and software.

The determination module 410 determines anatomic data 412 from a bone model 404. In certain embodiments, the system 400 may not include a determination module 410 if the anatomic data is available directly from the bone model 404. In certain embodiments, the anatomic data for a bone model 404 may include data that identifies each anatomic structure within the bone model 404 and attributes about the anatomic structure. For example, the anatomic data may include measurements of the length, width, height, and density of each bone in the bone model. Furthermore, the anatomic data may include position information that identifies where each structure, such as a bone is in the bone model 404 relative to other structures, including bones. The anatomic data may be in any suitable format and may be stored separately or together with data that defines the bone model 404.

In one embodiment, the determination module 410 may use advanced computer analysis system such as image segmentation to determine the anatomic data. The determination module 410 may determine anatomic data from one more sources of medical imaging data, images, files, or the like. Alternatively, or in addition the determination module 410 may use software and/or systems that implement one or more artificial intelligence methods (e.g., machine learning and/or neural networks) for deriving, determining, or extrapolating, anatomic data from medical imaging or the bone model. In one embodiment, the determination module 410 may perform an anatomic mapping of the bone model 404 to determine each unique aspect of the intended osteotomy procedure and/or bone resection and/or bone translation. The anatomic mapping may be used to determine coordinates to be used for an osteotomy procedure, position and manner of resections to be performed either manually or automatically or using robotic surgical assistance, a width for bone cuts, an angle for bone cuts, a predetermined depth for bone cuts, dimensions and configurations for resection instruments such as saw blades, milling bit size and/or speed, saw blade depth markers, and/or instructions for automatic or robotic resection operations.

In one embodiment, the determination module 410 may use advanced computer analysis system such as image segmentation to determine the anatomic data. The determination module 410 may determine anatomic data from one more sources of medical imaging data, images, files, or the like. The determination module 410 may perform the image segmentation using 3D modeling systems and/or artificial intelligence (AI) segmentation tools. In certain embodiments, the determination module 410 is configured to identify and classify portions of bone based on a condition of the bone, based on the bone condition. Such classifications may include identifying bone stability, bone density, bone structure, bone deformity, bone structure, bone structure integrity, and the like. Accordingly, the determination module 410 may identify portions or sections or one or more bones based on a quality metric for the bone. Advantageously, that determination module 410 can identify high quality bone having a viable structure, integrity, and/or density versus lower quality bone having a nonviable structure, integrity, and/or density and a plurality of bone quality levels in between.

Accordingly, the determination module 410 can guide a surgeon to determine which areas of one or more bones of a patient are within a “soft tissue envelope” (bone of undesirable quality) as that bone relates to a particular deformity or pathology. Identifying the quality of one or more bones of the patient can aid a surgeon in determining what type of correction or adjustment is needed. For example, an ulceration that occurs due to a boney deformity can be mapped using the determination module 410 in a way that a correction can be performed to correct the deformity and reduce pressure to an area and address the structures that were causing the pressure ulceration/skin breakdown.

In addition, the determination module 410 and/or another component of the apparatus 402 can be used to perform anatomic mapping which may include advanced medical imaging, such as the use of CT scan, ultrasound, MRI, X-ray, and bone density scans can be combined to effectively create an anatomic map that determines the structural integrity of the underlying bone.

Identifying the structural integrity of the underlying bone can help in determining where bone resections (e.g., osteotomies) can be performed to preserve the densest bone in relation to conditions such as Charcot neuropathic, arthropathy where lesser dense bone can fail and collapse. It is well documented in the literature that failure to address and remove such lesser dense bone can ultimately lead to failure of a reconstruction and associated hardware.

The present disclosure provides, by way of at least the exemplary system 400, an anatomic map that can be part of anatomic data. The anatomic map can combine structural, deformity, and bone density information and can be utilized to determine the effective density of bone and help to determine where bone should be resected in order to remove the lesser dense bone while maintaining more viable bone to aid in the planning of the osteotomy/bone resection placement.

The location module 420 determines or identifies one or more recommended locations and/or trajectory angles for deployment of an instrument, graft, and/or soft tissue based on the anatomic data 412 and/or the bone model 404. In one embodiment, the location module 420 may compare the anatomic data 412 to a general model that is representative of most patient's anatomies and may be free from deformities or anomalies. The location module 420 can operate autonomously and/or may facilitate input and/or revisions from a user. The location module 420 may be completely automated, partially automated, or completely manual. A user may control how automated or manual the determining of the location and/or trajectory angles is.

The provision module 430 is configured to provide a preliminary instrument model 438. The provision module 430 may use a variety of methods to provide the preliminary instrument model. In one embodiment, the provision module 430 may generate a preliminary instrument model. In the same, or an alternative embodiment, the provision module 430 may select a template instrument model for a tendon (or tendon substitute) deployment procedure configured to enable locating the position and/or providing the trajectory provided by the location module 420. In one embodiment, the provision module 430 may select a template instrument model for a minimally invasive surgical (MIS) bunion correction procedure configured to enable locating the position and/or providing the trajectory for the fixation deployment. In one embodiment, the provision module 430 may select a template instrument model from a set of template instrument models (e.g., a library, set, or repository of template instrument models).

The registration module 440 registers the preliminary instrument model with one or more bones or other anatomical structures of the bone model 404. As explained above, registration is a process of combining medical imaging data, patient imaging data, and/or one or more models such that the preliminary instrument model can be used with the bone model 404.

The design module 450 designs a patient-specific instrument (or patient-specific instrument model) based on the preliminary instrument model. The design operation of the design module 450 may be completely automated, partially automated, or completely manual. A user may control how automated or manual the designing of the patient-specific instrument (or patient-specific instrument model) is.

The manufacturing module 460 may manufacture a patient-specific instrument 406 using the preliminary instrument model. The manufacturing module 460 may use a patient-specific instrument model generated from the preliminary instrument model. The manufacturing module 460 may provide the patient-specific instrument model to one or more manufacturing tools and/or fabrication tool (e.g., additive and/or subtractive). The patient-specific instrument model may be sent to the tools in any format such as an STL file or any other CAD modeling or CAM file or method for data exchange. In one embodiment, a user can adjust default parameters for the patient-specific instrument such as types and/or thicknesses of materials, dimensions, and the like before the manufacturing module 460 provides the patient-specific instrument model to a manufacturing tool.

Effective connection of the guide to one or more bones can ensure that surgical steps are performed in desired locations and/or with desired orientations and mitigate undesired surgical outcomes.

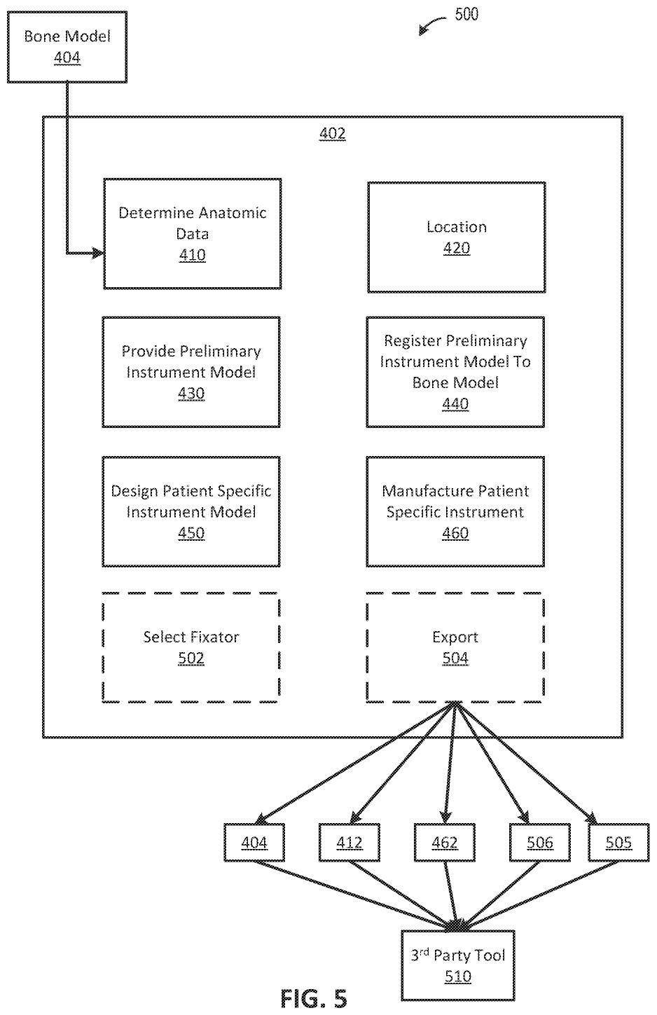

FIG. 5 illustrates an exemplary system 500 configured to generate one or more patient-specific instruments configured to correct a bone condition, according to one embodiment. The system 500 may include similar components or modules to those described in relation to FIG. 4. In addition, the system 500 may include a fixator selector 502 and/or an export module 504.

The fixator selector 502 enables a user to determine which fixator(s) to use for a MIS bunion correction procedure planned for a patient. In one embodiment, the fixator selector 502 may recommend one or more fixators based on the bone model 404, the location, the trajectory, or input from a user or a history of prior MIS bunion correction procedures performed. The fixator selector 502 may select a fixator model from a set of predefined fixator models or select a physical fixator from a set of fixators. The fixators may include a plate and associated accessories such as screws, anchors, and the like.

In one embodiment, the fixator selector 502 includes an artificial intelligence or machine learning module. The artificial intelligence or machine learning module is configured to implement one or more of a variety of artificial intelligence modules that may be trained for selecting fixator(s) based on anatomic data 412 and/or other input parameters. In one embodiment, the artificial intelligence or machine learning module may be trained using a large data set of anatomic data 412 for suitable fixator(s) identified and labeled in the dataset by professionals for use to treat a particular condition. The artificial intelligence or machine learning module may implement, or use, a neural network configured according to the training such that as the artificial intelligence or machine learning module is able to select or recommend suitable fixator(s).

The export module 504 is configured to enable exporting of a patient-specific instrument model 462 for a variety of purposes including, but not limited to, fabrication/manufacture of a patient-specific instrument 406 and/or fixator(s), generation of a preoperative plan, generation of a physical bone model matching the bone model 404, and the like. In one embodiment, the export module 504 is configured to export the bone model 404, anatomic data 412, a patient-specific instrument model 462, a preoperative plan 506, a fixator model 508, or the like. In this manner the custom instrumentation and/or procedural steps for a procedure (e.g., a graft harvesting procedure, minimally invasive surgical (MIS) procedure, or the like) can be used in other tools. The preoperative plan 506 may include a set of step by step instructions or recommendation for a surgeon or other staff in performing a procedure (e.g., a graft harvesting procedure, minimally invasive surgical (MIS) procedure, or the like). The preoperative plan 506 may include images and text instructions and may include identification of instrumentation to be used for different steps of the procedure (e.g., a graft harvesting procedure, minimally invasive surgical (MIS) procedure, or the like). The instrumentation may include the patient-specific instrument 406 and/or one or more fixators/fasteners. In one embodiment, the export module 504 may provide a fixator model which can be used to fabricate a fixator for the procedure.

The exports (404, 412, 462, 506, and 508) may be inputs for a variety of 3rd party tools 510 including a manufacturing tool, a simulation tool, a virtual reality tool, an augmented reality tool, an operative procedure simulation tool, a robotic assistance tool, and the like. A surgeon can then use these tools when performing a procedure or for rehearsals and preparation for the procedure. For example, a physical model of the bones, patient-specific instrument 406, and/or fixators can be fabricated, and these can be used for a rehearsal operative procedure. Alternatively, a surgeon can use the bone model 404, preliminary instrument model 438, and/or a fixator model to perform a simulated procedure using an operative procedure simulation tool.

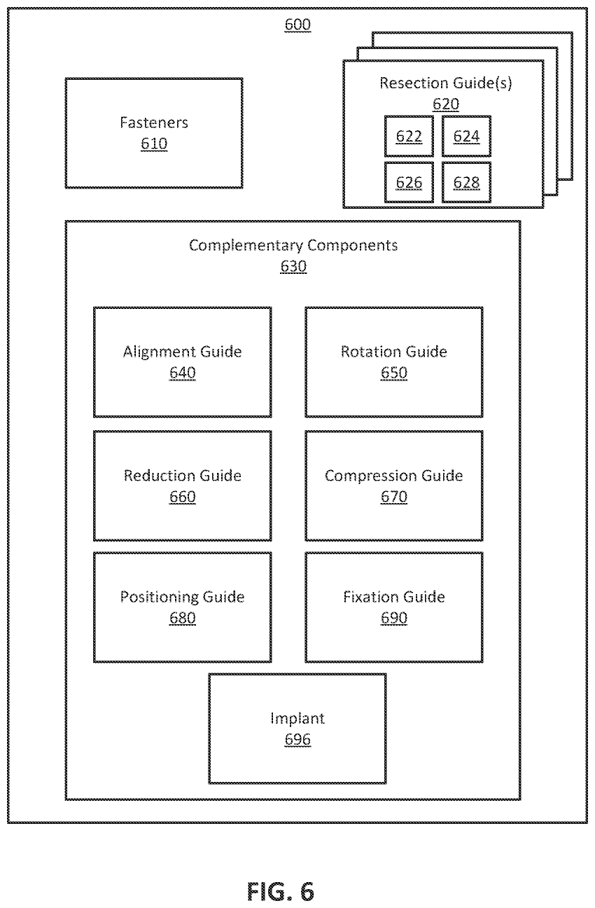

FIG. 6 illustrates an exemplary system 600, according to one embodiment. The system 600 can include one or more fasteners 610, one or more resection guides 620, and one or more complementary components 630. While a system 600 can be used for a variety of procedures, one or more features, components, and/or aspects of the system 600 may be particularly suited for one or more osteotomies on one or more bones of a structure such as a patient's foot, ankle, wrist, hand, shoulder, or the like.

In certain embodiments, the one or more fasteners 610 can include one or more permanent fasteners and/or one or more temporary fasteners. Typically, the fasteners 610 may be used during a variety of different steps of a procedure. Temporary fasteners are often used because they can securely hold bone or parts/fragments of bones while steps of the procedure are conducted. A common temporary fastener that can be used with system 600 is a K-wire, also referred to as a pin, guide pin, and/or anchor pin.

The one or more resection guides 620 assist a surgeon in performing different resection or dissection steps for an osteotomy or other procedure. In certain embodiments, a resection guide 620 includes one or more resection features 622 and one or more bone attachment features 624. The resection features 622 can take a variety of forms and/or embodiments. In one embodiment, the resection features 622 take the form of a cut channel or slot or other opening. Similarly, the bone attachment features 624 can take a variety of forms and/or embodiments. The resection features 622 provide a guide for a surgeon using a cutting tool to resect a bone, one or more bones, or other tissues of a patient. In certain embodiments, the resection features 622 may guide a surgeon in performing a resection, and osteotomy, and/or a dissection. The bone attachment features 624 serve to secure the resection guide 620 to one or more bones and/or one or more other structures. Often, a bone attachment feature 624 can include a hole in the resection guide 620 together with a temporary fastener such as a K-wire or pin.

The bone attachment features 624 facilitate attachment of a resection guide 620 to one or more bones, or bone fragments, of a patient. The bone attachment features 624 may include any of a wide variety of fasteners including, but not limited to, holes, spikes, fastening devices, and/or the like. Effective connection of the resection guide 620 to one or more bones across a joint and/or to one or more bones can ensure that cut surfaces are formed in desired locations and orientations and mitigate removal of hard tissue and/or soft tissue in undesired locations and/or orientations.

In certain embodiments, a resection guide 620 may include one or more bone engagement surfaces 626 and/or one or more landmark registration features 628. In certain embodiments, a landmark registration feature 628 may extend from one or more sides of the resection guide 620 and engage with one or more landmarks of a bone or joint or anatomical structure of a patient. Registration of the landmark registration feature 628 to a landmark of a bone can serve to confirm that a surgeon has located a desired placement and/or orientation for a resection guide 620.

In certain embodiments, the bone engagement surfaces 626 are patient-specific: contoured to match a surface of: one or more bones the resection guide 620 contacts during the procedure or one or more joints proximal to the resection guide 620 during the procedure. Alternatively, or in addition, the bone engagement surface 626 may not be patient-specific, and may, or may not, contact a bone surface during use of the resection guide 620. In one embodiment, a skin contact surface may be used in addition to or in place of a bone engagement surface. Those of skill in the art appreciate that one or more sides of any of the members of the system 600 may include one or more bone engagement surfaces 626. Consequently, one or more sides of the fasteners 610, the resection guide(s) 620, the complementary components 630, and/or the implants 696 may include one or more bone engagement surfaces 626.

In certain embodiments, the resection guide s 620 and/or aspects of the resection guide s 620 may be integrated into other components and/or instruments, such as a pin guide, a trajectory guide, an alignment guide or the like.

The complementary components 630 serve to assist a surgeon during one or more steps of a procedure. Those of skill in the art appreciate that a number of components can serve as complementary components 630. One or more of the features, functions, or aspects of the complementary components 630 can include patient-specific features.

Examples of complementary components 630 include, but are not limited to, an alignment guide 640, a rotation guide 650, a reduction guide 660, a compression guide 670, a positioning guide 680, a fixation guide 690, and/or one or more implants 696. In general, the complementary components 630 serve to assist a surgeon in performing the function included in the name of the complementary component 630. Thus, an alignment guide 640 can help a surgeon align bones, parts of bones, or other parts of a patient as part of a procedure. A rotation guide 650 can help a surgeon rotate one or more bones, parts of bones, or other parts of a patient as part of a procedure. In one embodiment, a rotation guide 650 may hold one bone fragment stable while another bone fragment is rotated into a desired position.

A reduction guide 660 can help a surgeon position and/or orient one or more bones, parts of bones, or other parts of a patient as part of a procedure in order to reduce the bone, bones, bone parts, or other parts and/or in order to position and/or orient the bone, bones, bone parts, or other parts to a desired position and/or orientation. In certain embodiments, aspects and/or features of a reduction guide 660 can be integrated into one or more other components of an osteotomy system 600, such as components of the complementary components 630. A compression guide 670 can help a surgeon compress one or more bones, parts of bones, or other parts of a patient together or against an implant as part of a procedure. A positioning guide 680 (also referred to as a positioner) can help a surgeon position one or more bones, parts of bones, or other parts of a patient as part of a procedure. For example, a positioning guide 680 may hold one bone or bone fragment stable and hold one or more other bone fragments in a desired position while permanent or temporary fixation is deployed. In certain embodiments, the positioning guide 680 may hold bone fragments in a reduced position, and thus may function as both a positioning guide 680 and/or a reduction guide 660.

In certain embodiments, the positioning guide 680 may be designed and fabricated to be patient-specific. The patient-specific aspects can include a patient-specific bone engagement surface, a predefined angle for reorienting one or more bone or bone parts within one or more planes, a predefined position for bone attachment features 624 or fasteners 610, a predefined or patient-specific offset or amount of translation that is provided, or the like. Alternatively, or in addition, the positioning guide 680 may be selected from a kit, collection, or repository of a number of positioning guides 680: each having a different configuration for one or more aspects/attributes of the positioning guide 680. For example, each member of the repository/kit may include a different positioning angle (repositioning or correction angle), the angles may differ by 2 degrees for example. In such an embodiment, each positioning guide 680 may not be patient-specific to a particular patient but may provide the desired amount of positioning to meet the goals of the surgeon. In certain embodiments, a preoperative plan generated based on the present disclosure may include a recommendation for the positioning guide 680 to be used, even if the recommended positioning guide 680 is not patient-specific to the particular patient.

A fixation guide 690 can help a surgeon in completing one or more temporary or permanent fixation steps for one or more bones, parts of bones, or other parts of a patient as part of a procedure. The fixation guide 690 may include and/or may use one or more components of a fastener or fixation system including implant hardware of the fastener or fixation system.

One example of a complementary components 630 may include a compressor/distractor. The compressor/distractor can be used to compress or distract bones or parts of bones involved in a procedure.

Advantageously, the system 600 can help a surgeon overcome one or more of the challenges in performing an osteotomy procedure, particularly on bones of a hand or of a foot of a patient, such as on the forefoot, midfoot, or hindfoot. One challenge during an osteotomy procedure can be maintaining control of, and/or position, and/or orientation of a bone, one or more bones, and/or bone pieces/fragments, particularly once a resection or dissection is performed. Advantageously, the fasteners 610, resection guide(s) 620, and/or complementary components 630 can be configured to assist in overcoming this challenge.

Advantageously, the system 600 can help a surgeon in positioning, placing, and/or orienting a resection guide accurately. Modern techniques may include preoperative planning, simulation, or even practice using computer models, 3D printed models, virtual reality systems, augmented reality systems or the like. However, simulations and models are still different from actually positioning a resection guide on a patient's bone, joint, or body part during the procedure. The system 600 can include a number of features, including patient-specific features, to assist the surgeon with the positioning. In one embodiment, the resection guide 620 can include one or more landmark registration features 628.

Advantageously, the system 600 can help a surgeon in securing guides of the osteotomy system 600, such as a resection guide, as well as how to readily remove the guide (e.g., resection guide) without disturbing a reduction, shifting, reorienting, or repositioning one or more bones or parts of bones while removing the guide. In certain embodiments, the system 600 is configured to permit removal of a guide while keeping temporary fasteners in place for use in subsequent steps of an osteotomy procedure. Alternatively, or in addition, the system 600 may facilitate positioning of temporary fasteners during one step of a wedge osteotomy procedure for use in a subsequent step of the wedge osteotomy procedure. Removal of a guide during an osteotomy procedure can be particularly challenging where translation and/or rotation of the bones involved in the osteotomy procedure is required for the success of the osteotomy procedure. Advantageously, the system 600 accommodates translation and/or rotation of the bones during the osteotomy procedure while facilitating a successful outcome for the osteotomy procedure.

Advantageously, the components of the system 600 can be specifically designed for a particular patient. Alternatively, or in addition, the components of the system 600 can be specifically designed for a class of patients. Each of the components of the system 600 can be designed, adapted, engineered and/or manufactured such that each feature, attribute, or aspect of the component is specifically designed to address one or more specific indications present in a patient. Advantageously, the cuts made for the osteotomy procedure can be of a size, position, orientation, and/or angle that provides from an optimal osteotomy with minimal risk of undesirable resection. In one embodiment, the components of the system 600 can be configured such that an osteotomy is performed that enables a correction in more than one plane in relation to the parts of the body of the patient. For example, cut channels or resection features 622 in a resection guide 620 can be oriented and configured such that when the bones are fused/fixated the correction results from translation, rotation, and/or movement of bones or bone parts in two or more planes (e.g., sagittal and transverse) once the fragments or bones are reduced.

In certain embodiments, the exemplary system 600 may include a plurality of fasteners 610, resection guides 620, and/or complementary components 630. For example, a surgeon may plan to resect a plurality of osteotomies from the bone(s) in order to accomplish a desired correction. In one example, one or more wedge segments may be resected from a medial side of a patient's foot and another one or more wedge segments may be resected from a lateral side of the patient's foot. These wedge segments may extend part way into the foot, or through from one side of the foot to the other. Of course, multiple wedge segments may be formed on one side of the foot as well.

Additionally, a surgeon may use one or more components in an exemplary system 600 to make multiple cuts in the bone(s). The multiple cuts may be centered over or around an apex of a deformity or positioned at other locations within the foot such that when the multiple cuts are made, any resected segments removed, or added bone void fillers introduced, and/or bones and/or bone fragments translated and/or rotated the combined angles, surfaces, removed segments, and/or added portions cooperate to provide a desired correction. Each of the components of the exemplary system 600 can be identified, defined, and reviewed using the apparatuses, systems, and/or methods of the present disclosure.

In certain embodiments, the components of the system 600 may be made as small as possible to minimize the amount of soft tissue that is opened in the patient for the osteotomy procedure. Alternatively, or in addition, walls and/or sides of the components may be beveled and/or angled to avoid contact with other hard tissue or soft tissues in the operating field for the osteotomy procedure.

Those of skill in the art will appreciate that for certain osteotomy procedure a complementary component 630 may not be needed or a given complementary component 630 may be optional for use in the osteotomy procedure. Similarly, those of skill in the art will appreciate that certain features of the fasteners 610, resection guides 620, and/or complementary components 630 can be combined into one or more of apparatus or devices or may be provided using a plurality of separate devices.

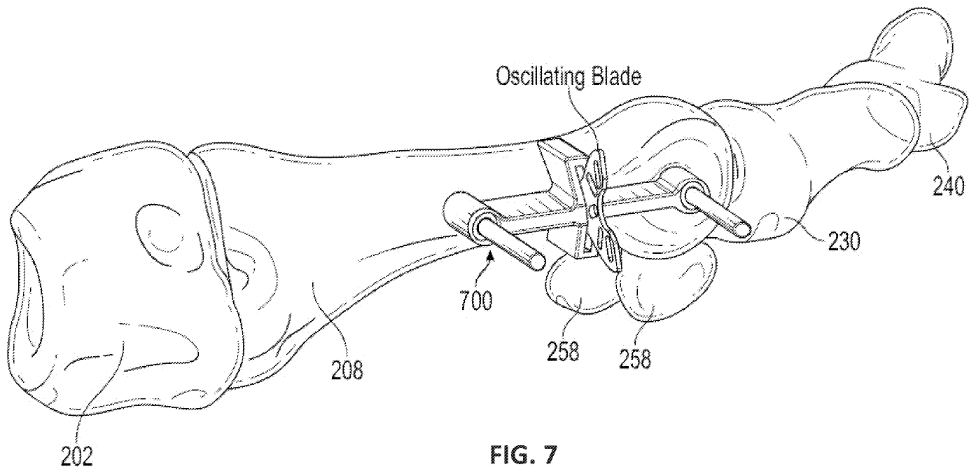

FIG. 7 is a side perspective view of a resection guide 700 (also referred to as a cutting guide 700 according to one embodiment) shown secured to a bone. In this example, the resection guide 700 is secured to a medial side of a first metatarsal 208. The first metatarsal 208 includes two sesamoids 258. Of course, the resection guide 700 can be secured to other bones of a patient to guide a surgeon in making cuts or performing one or more osteotomies in one or more bones. The resection guide 700 may also be referred to as an oscillating blade cut jig based on the type of cutting tool the resection guide 700 is designed to accept.

When secured as illustrated in FIG. 7, a user may operate the resection guide 700 by inserting a planar cutting tool 760 such as a rectangular oscillating blade attached to a manual, mechanical, pneumatic, or electric driver into a slot or channel or guide feature in a cutting guide. Next, the user may cut into the bone.

In certain embodiments, the slot or channel or guide feature is larger than a distal end of the rectangular oscillating blade. Consequently, a user can pivot the rectangular oscillating blade such that the rectangular oscillating blade is directed dorsally, in relation to the bone, as the rectangular oscillating blade aims and moves dorsally, the rectangular oscillating blade cuts the bone towards the dorsal surface of the bone. Similarly, the user can pivot the rectangular oscillating blade such that the rectangular oscillating blade is directed plantarly, in relation to the bone, as the rectangular oscillating blade aims and moves plantarly, the rectangular oscillating blade cuts the bone towards the plantar surface of the bone. In this manner, a user can resection a bone.

Advantageously, such resections can be done using very small incisions, incisions just large enough to insert the resection guide 700 and engage with, anchor to, the bone. Alternatively, or in addition, the resection guide 700 may contact or register with the medial skin surface and a narrow incision may be made to accept the distal end of the oscillating blade. In this manner, a majority of the bone resection can be done subcutaneously. Accordingly, the resection guide 700 provides a minimally invasive surgical (MIS) instrument for use in osteotomies.

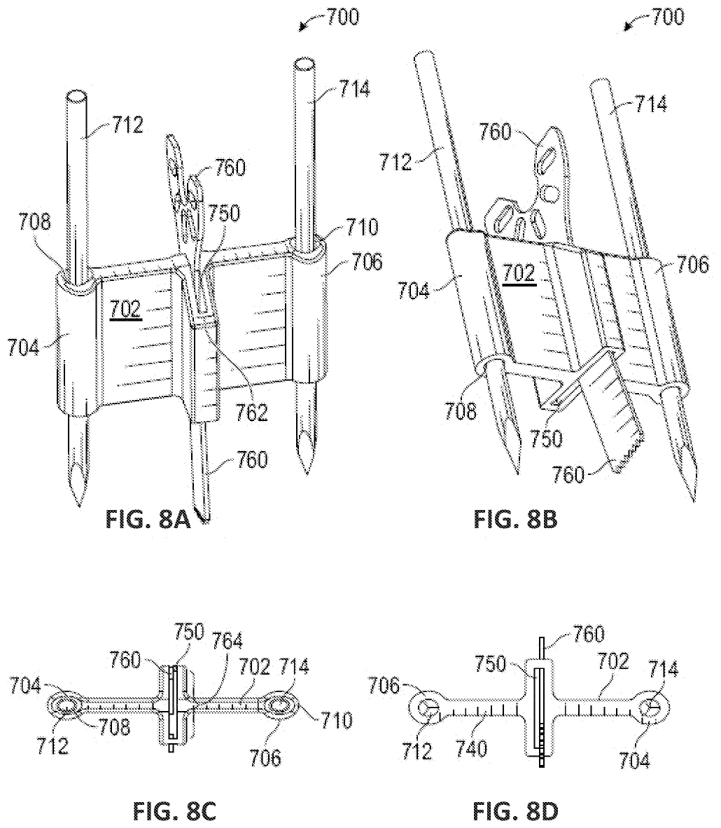

FIGS. 8A-8D are a top perspective, bottom perspective, top, and bottom, views respectively of the resection guide of FIG. 7.

FIG. 8A is a top perspective view of the resection guide 700 of FIG. 7. The resection guide 700 may include a body 702 having a proximal end 704 and a distal end 706. The proximal end 704 and/or distal end 706 may each include at least one hole, a proximal pin hole 708 and a distal pin hole 710 configured to receive a fastener. The proximal pin hole 708 may be configured to receive a proximal pin 712. The distal pin hole 710 may be configured to receive a distal pin 714.

The body 702 includes a guide feature that can be implemented as a slot, a hole, or a cut channel 750 in the body 702. FIG. 8A illustrates a cutting tool 760, such as an oscillating blade that is configured to fit within the cut channel 750. In certain embodiments, the cut channel 750 may include an opening, such as a slot that includes a width 762 that is greater than the thickness of the cutting tool 760. In certain embodiments, the width 762 may be just greater than the thickness of an oscillating blade that is configured to fit within the cut channel 750. In another embodiment, the width 762 may be greater than a thickness of another cutting tool 760 such as a rotary tool such as a surgical bur. In such an embodiment, the cut channel 750 may serve as a boundary for performing an osteotomy on the bone, (e.g., a metatarsal). In one embodiment, the cut channel 750 is positioned to guide resection of the first metatarsal for the osteotomy that forms the a capital fragment from a head of a long bone. Often the cut channel 750 is perpendicular to a long axis of the bone being cut. Alternatively, or in addition, the cut channel 750 may have other angles or orientations relative to the long axis of the bone being cut.

In certain embodiments, the cut channel 750 may include an opening, such as a slot that includes a length 764 that is greater than a width of the cutting tool 760. In certain embodiments, the length 764 may be just greater than the thickness of an oscillating blade or another cutting tool 760 such as a rotary tool such as a surgical bur. Having the length 764 greater than the cutting tool 760 can enable a surgeon to perform an osteotomy on the bone, (e.g., a metatarsal) by pivoting the cutting tool 760 near the cut channel 750 in one or both of a dorsal direction and/or a plantar direction. Such pivoting use of the cut channel 750 allows the resection guide 700 to be of minimal size while the surgeon performs resection subcutaneously.

In certain embodiments, the cut channel 750 may be positioned between the proximal end 704 and the distal end 706 based on patient imaging data. Thus, the position of the cut channel 750 may be in a center between the proximal end 704 and the distal end 706. Alternatively, or in addition, the cut channel 750 may be closer to the proximal end 704 than the distal end 706. Alternatively, or in addition, the cut channel 750 may be closer to the distal end 706 than the proximal end 704. Similarly, the size and angle of the cut channel 750 relative to a longitudinal axis of the body 702 and/or an orientation of the cut channel 750 relative to a longitudinal axis of a bone that is to be resected may be defined based on patient imaging data.

FIG. 8B is a bottom perspective view of the resection guide 700 of FIG. 7.

FIG. 8C is a top view of the resection guide 700 of FIG. 7.

FIG. 8D is a bottom view of the resection guide 700 of FIG. 7. FIG. 8D illustrates an embodiment of the resection guide 700 that includes a body 702 having an inferior surface 740 that can be planar. In certain embodiments, the inferior surface 740 may not be contoured to substantially match a surface contour of bone (e.g., a metatarsal) where the resection guide 700 is to be anchored/secured.

In another embodiment, the inferior surface 740 may be contoured to substantially match a surface contour of bone (e.g., a metatarsal) where the resection guide 700 is to be anchored/secured. In such an embodiment, the inferior surface 740 may be contoured based on patient imaging data of the bone involved in an osteotomy procedure. Alternatively, or in addition, the inferior surface 740 may be contoured based on an expected contour for a bone that will be used with the resection guide 700. Alternatively, or in addition, the inferior surface 740 may be contoured based on placement of the resection guide 700 on a surface of skin of a patient for a procedure.

In certain embodiments, the proximal end 704 and distal end 706 may straddle a joint with the proximal end 704 on one side of the joint and the distal end 706 on the other side of the joint. In another embodiment, the proximal end 704 and distal end 706 may be secured along a length of the same bone.

In certain embodiments, where the resection guide 700 that includes a body 702 having an inferior surface 740 that is planar, a registration process may be used to position the resection guide 700 in a desired position by registering on a surface of a patient's skin near a bone to be resected, such as medial side of a metatarsal. Advantageously, having a high degree of detail from the medical imaging permits the models to have a high degree of detail that is reflected in patient-specific instrumentation generated from the models. The patient-specific instrumentation then more readily registered to the surface of one or more bone(s) and/or the surface of the skin of the patient to facilitate desired placement, positioning, and orientation of the patient-specific instrumentation.

Those of skill in the art will appreciate that a variety of surgical techniques may be used with the resection guide 700 as well as with other components described herein. In such surgical techniques, placement of these other guides may rely on proper placement of the proximal pin 712 or proper placement of the distal pin 714 that goes into a distal end of a bone, such as a distal end of a metatarsal that is referred to as a capital fragment after the osteotomy. Placement of the distal pin 714 in the capital fragment and/or a proximal pin 712 in another part of a bone can serve as a reference point for one or more other guides used in a surgical technique such that a desirable outcome can be achieved.

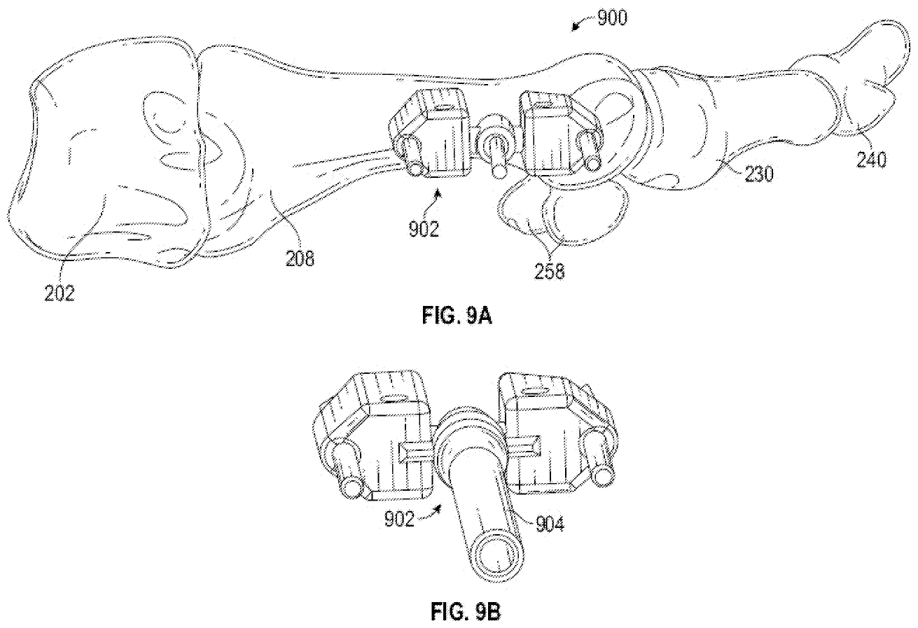

FIG. 9A is a side perspective view of an osteotomy system 900 according to one embodiment. The osteotomy system 900 includes a pivoting resection guide 902 (which may be also referred to as a swiveling resection guide) according to one embodiment, shown secured to a bone. In this example, the pivoting resection guide 902 is secured to a metatarsal bone. Of course, the pivoting resection guide 902 can be secured to other bones of a patient to guide a surgeon in making cuts in one or more bones. The pivoting resection guide 902 may also be referred to as a pivoting cut jig, swiveling resection guide, rotating resection guide, arc resection guide, or a pivoting cutting guide based on how the pivoting resection guide 902 operates.

When secured as illustrated in FIG. 9A, a user may operate the pivoting resection guide 902 by inserting a cutting tool such as a burr, a burr drill bit, or a drill bit attached to a manual, mechanical, pneumatic, or electric driver into a hole in the guide 902. Next, the user may cut into the bone. In the case of a drill bit with longitudinal flutes, the drill bit may initially drill a hole in the bone. Next, a user can pivot the pivoting resection guide 902 such that the drill bit (specifically a distal end of the drill bit) moves dorsally in relation to the bone, as the cutter guide pivots, the drill bit cuts bone in the dorsal direction. Similarly, the user can pivot the cutter guide such that the drill bit moves plantarly in relation to the bone, as the cutter guide pivots, the drill bit cuts bone in the plantar direction. In this manner, a user can resection a bone.

Advantageously, such resections can be done using very small incisions, incisions just large enough to insert the drill bit. Alternatively, or in addition, such resections can be done using incisions just large enough to insert the pivoting resection guide 902 and engage the pivoting resection guide 902 the bone. In certain embodiments, the bone resection can be done subcutaneously. In other embodiments, bone resection can be done percutaneously through an incision made by of made for the drill bit while the pivoting resection guide 902 presses against a surface of the skin at the resection location. Accordingly, the pivoting resection guide 902 provides a minimally invasive surgical (MIS) instrument for use in osteotomies. Those of skill in the art will appreciate that while a small puncture incision may work initially for use of a pivoting resection guide 902 a larger incision may be needed to accept other instruments that will be used to complete a surgical procedure.

FIG. 9B is a perspective view of a cutting guide, such as pivoting resection guide 902, according to one embodiment, that includes a handle 904. The handle 904 may engage the pivoting resection guide 902 and may serve to facilitate pivoting or swiveling the pivoting resection guide 902 while resecting. A surgeon may move the handle 904 to direct the resection.

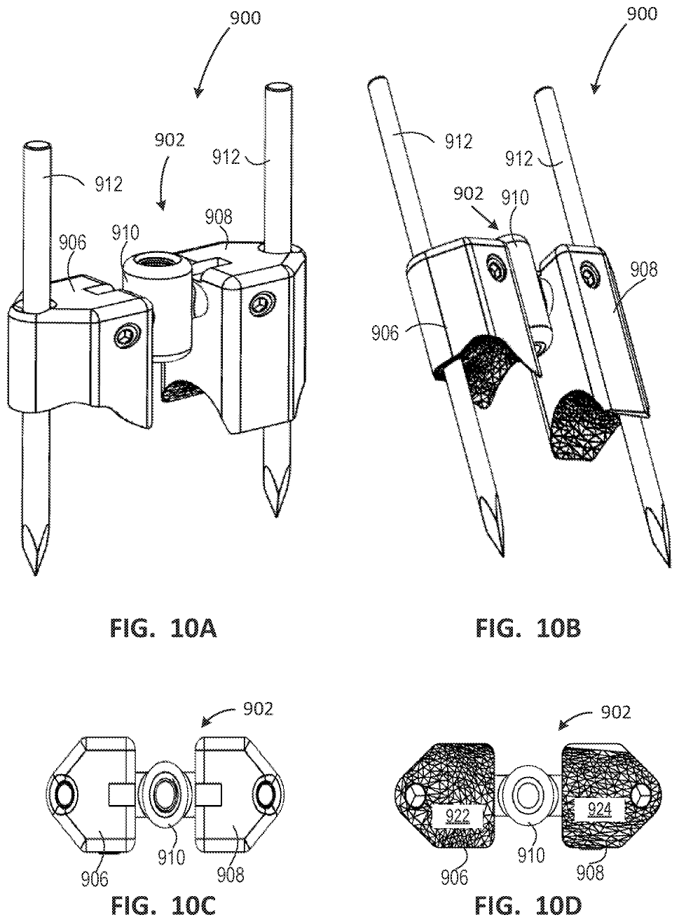

FIG. 10A is a top perspective view of a pivoting resection guide 902, according to one embodiment. The pivoting resection guide 902 may include one or more bone attachment features and a cutter guide 910 configured to pivot or swivel about an axis transverse to a long axis of the cutter guide 910.

In the illustrated embodiment, the pivoting resection guide 902 includes a first bone attachment feature 906, a second bone attachment feature 908, and a cutter guide 910 positioned between the first bone attachment feature 906 and the second bone attachment feature 908. The first bone attachment feature 906 and/or second bone attachment feature 908 may include one or more holes, each hole configured to receive a pin such as a fastener 916. The first bone attachment feature 906 and/or second bone attachment feature 908 are configured to secure or anchor the pivoting resection guide 902 to one or more bones of a patient. The cutter guide 910 is configured to accept a cutting tool and guide the cutting tool along a predetermined trajectory as the cutter guide 910 swivels or pivots about an axis (e.g., an axis transverse to a long axis of the cutter guide 910).

FIG. 10B is a bottom perspective view of the pivoting resection guide 902, according to one embodiment.

FIG. 10C is a top view of the pivoting resection guide 902, according to one embodiment.

FIG. 10D is a bottom view of the pivoting resection guide 902, according to one embodiment. FIG. 10D illustrates an embodiment of the pivoting resection guide 902 that includes a first bone attachment feature 906 having a first bone engagement surface 922 that is contoured to substantially match a surface contour of bone (e.g., a proximal metatarsal surface contour) where the pivoting resection guide 902 is to be anchored/secured. In certain embodiments, the pivoting resection guide 902 can include a second bone attachment feature 908 having a second bone engagement surface 924 that is contoured to substantially match a surface contour of bone (e.g., a distal metatarsal surface contour) where the pivoting resection guide 902 is to be anchored/secured. Alternatively, or in addition, the first bone engagement surface 922 and/or second bone engagement surface 924 may be contoured based on placement of the pivoting resection guide 902 on a surface of skin of a patient for a procedure.

In certain embodiments, the inferior surfaces of the first bone attachment feature 906 and/or the second bone attachment feature 908 may be planar and may not be configured to conform, or substantially match a surface contour of bone (e.g., a metatarsal) where the pivoting resection guide 902 is to be anchored/secured. Alternatively, or in addition, the inferior surfaces of the first bone attachment feature 906 and/or the second bone attachment feature 908 may be contoured to sit on a surface of skin of a patient that covers the bone (e.g., a metatarsal).

In certain embodiments, the first bone attachment feature 906 and second bone attachment feature 908 may straddle a joint with the first bone attachment feature 906 on one side of the joint and the second bone attachment feature 908 on the other side of the joint. In another embodiment, the first bone attachment feature 906 and second bone attachment feature 908 may be secured along a length of the same bone.

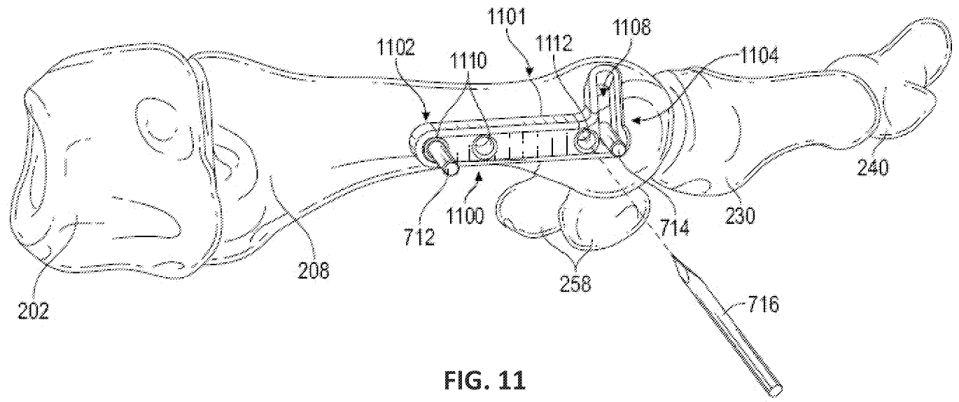

FIG. 11 is a side perspective view of a rotation guide 1100 according to one embodiment, shown secured to a bone. In certain embodiments, the rotation guide 1100 may also be referred to as an angular pin guide. In the example of FIG. 11, the rotation guide 1100 is secured to a first metatarsal 208 bone. Of course, the rotation guide 1100 can be secured to other bones of a patient to guide a surgeon in repositioning and/or reorienting one or more bones. The rotation guide 1100 may also be referred to as a de-rotation guide because the rotation guide 1100 can be used to rotate or de-rotate a whole bone or a segment of bone.

When secured as illustrated in FIG. 11, a user may use the rotation guide 1100 after resecting the bone. In the illustrated example, a user has resected a metatarsal bone (e.g., first metatarsal 208), separating a metatarsal head from the shaft of the metatarsal, the metatarsal head separated from the shaft becomes a capital fragment. The user may want to rotate (or de-rotate since the bone or bone segment may have rotated to an unnatural orientation) a bone or bone segment, such as a head (aka capital fragment) of a metatarsal bone to a preferred and/or more optimal rotational position relative to the shaft. In one embodiment, the user may rotate the capital fragment to place the sesamoids 258 on the plantar side of the foot.

Advantageously, a user can secure the rotation guide 1100 to the metatarsal bone across the resected portion 1101. In one embodiment, a user can place the rotation guide 1100 over the same pins used when for performing the osteotomy of the first metatarsal 208 to form the capital fragment. For example, after the osteotomy the proximal pin 712 and distal pin 714 remain in the bone fragments. A surgeon can slide the rotation guide 1100 over the proximal pin 712 on the proximal end 1102, by way of one of the holes 1110 and the distal pin 714 on the distal end 1104 by way of the rotation slot 1108. In one embodiment, inserting the distal pin 714 through the rotation slot 1108 may draw the capital fragment towards the cut face of the shaft and close a gap formed by an osteotomy. The rotation slot 1108 enables rotation of the capital fragment relative to the first metatarsal.

Having placed the proximal pin 712 and distal pin 714, earlier before the osteotomy, and now using these same pins with the rotation guide 1100 gives the surgeon control over the position of each bone fragment relative to the other. With the distal pin 714 in the rotation slot 1108 a surgeon can rotate the capital fragment as desired to a desired position.

In one embodiment, the rotation guide 1100 may be secured to the bone using two or more proximal pins that pass through the one or more holes 1110 positioned towards the proximal end 1102. Those of skill in the art will appreciate that the rotation guide 1100 can have a shorter or a longer rotation slot 1108. Alternatively, or in addition, a user may choose a rotation guide 1100 from a kit where each rotation guide 1100 has a different length rotation slot 1108 to allow for more or less rotation of the capital fragment.

With the rotation guide 1100 secured to the bone, a user can then rotate a bone segment about a longitudinal axis of the bone by moving the distal pin 714 within the rotation slot 1108. Advantageously, once a desired rotated position of the bone segment is achieved, a user may secure the rotated position by inserting a fastener, such as another pin, referred to herein as an anchor pin 716, into an anchor hole 1112. The anchor hole 1112 is configured to receive the anchor pin 716 that is deployed in the bone segment (e.g., capital fragment) having a desired rotated position relative to the first metatarsal. With an anchor pin 716 in anchor hole 1112, the bone segment is secured in a desired rotated position.

In certain embodiments, the rotation slot 1108 may be positioned anywhere between, or at, the proximal end 1102 and the distal end 1104. In one embodiment, the rotation slot 1108 may be sized, positioned, and/or oriented based on patient imaging data.

In one embodiment, the position of the rotation slot 1108 may be near a center between the proximal end 1102 and the distal end 1104. Alternatively, or in addition, the rotation slot 1108 may be closer to the proximal end 1102 than the distal end 1104. Alternatively, or in addition, the rotation slot 1108 may be closer to the distal end 1104 than the proximal end 1102. Similarly, the size, length, width, and angle of the rotation slot 1108 relative to the rotation guide body 1114 and/or an orientation of the rotation slot 1108 relative to a longitudinal axis of a bone may be defined based on patient imaging data. Alternatively, or in addition, a plurality of rotation guides 1100 may be provided that each enable a different number of degrees of rotation of the capital fragment in relation to the metatarsal.

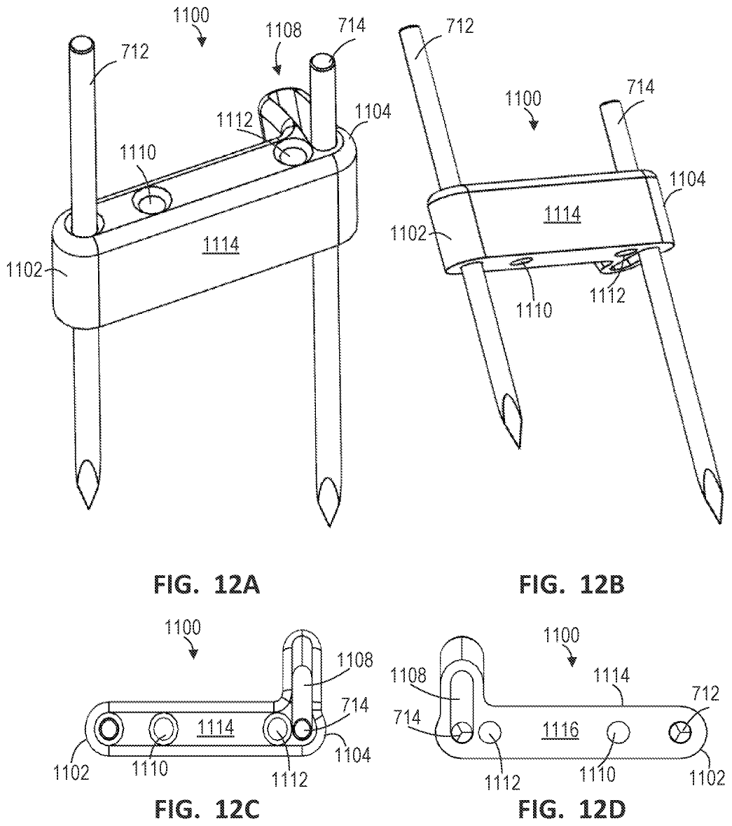

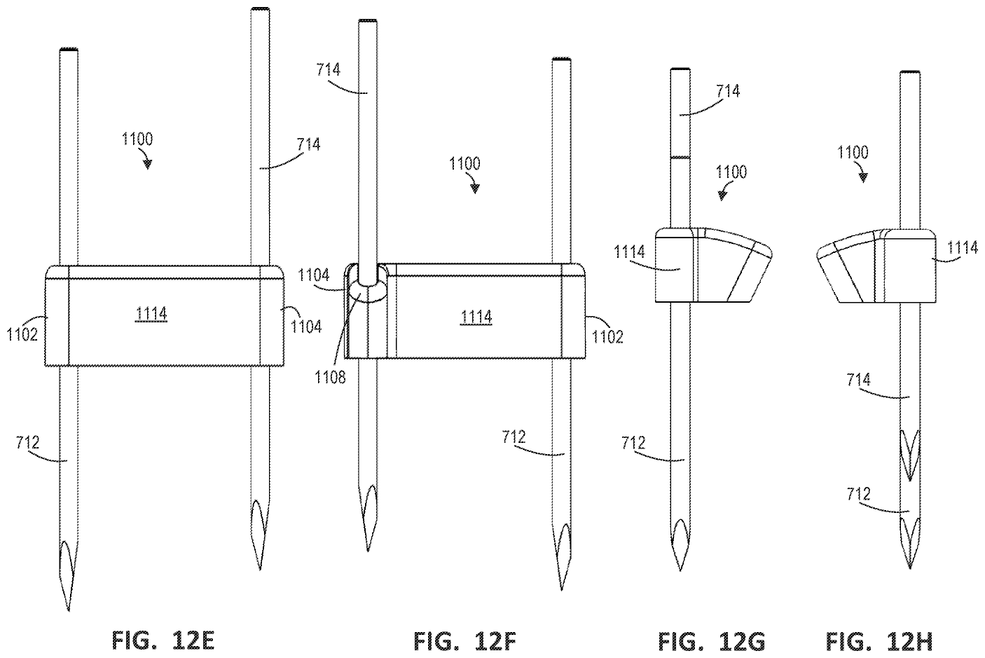

FIG. 12A is a top perspective view of the rotation guide 1100 of FIG. 11. The rotation guide 1100 includes a rotation guide body 1114. The rotation guide body 1114 may include at least two pin holes configured to engage two or more pins. The rotation guide body 1114 may include a proximal end and a distal end.

FIG. 12B is a bottom perspective view of the rotation guide 1100 of FIG. 11.

FIG. 12C is a top view of the rotation guide 1100 of FIG. 11.

FIG. 12D is a bottom view of the rotation guide 1100 of FIG. 11. FIG. 12D illustrates an embodiment of the rotation guide 1100 that includes a rotation guide body 1114 having an inferior surface 1116 that can be planar. In certain embodiments, the inferior surface 1116 may not be contoured to substantially match a surface contour of bone (e.g., a metatarsal) where the rotation guide 1100 is to be anchored/secured. In another embodiment, the inferior surface 1116 may be contoured to substantially match a surface contour of bone (e.g., a metatarsal) where the rotation guide 1100 is to be anchored/secured. In such an embodiment, the inferior surface 1116 may be contoured based on patient imaging data of the bone involved in an osteotomy procedure. Alternatively, or in addition, the inferior surface 1116 may be contoured based on an expected contour for a bone that will be used with the rotation guide 1100, such that the inferior surface 1116 may have a general shape or curve or contour similar to that of the surface of the bone, but not an exact match. Alternatively, or in addition, the inferior surface 1116 may be contoured based on placement of the rotation guide 1100 on a surface of skin of a patient for a procedure.

In certain embodiments, the proximal end 1102 and distal end 1104 may straddle a joint with the proximal end 1102 on one side of the joint and the distal end 1104 on the other side of the joint. In another embodiment, the proximal end 1102 and distal end 1104 may be secured along a length of the same bone.

FIG. 12E is a front view of the rotation guide 1100 of FIG. 11.

FIG. 12F is a back view of the rotation guide 1100 of FIG. 11.

FIG. 12G is a left side view of the rotation guide 1100 of FIG. 11.

FIG. 12H is a right side view of the rotation guide 1100 of FIG. 11.

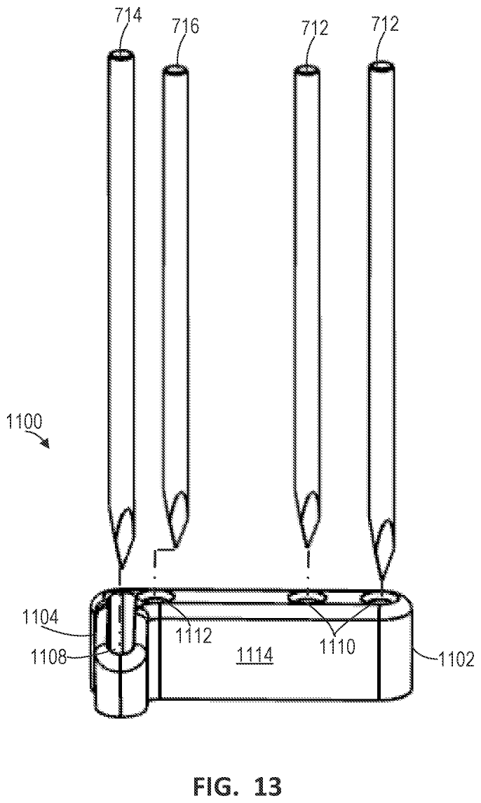

FIG. 13 is an exploded view of the rotation guide 1100 of FIG. 11. FIG. 13 illustrates two proximal pins 712, a distal pin 714, and an anchor pin 716. In one embodiment, the anchor pin 716 is inserted after the distal pin 714 is inserted and moved within the rotation slot 1108 to a desired position. Once the anchor pin 716 is inserted, the distal pin 714 can be removed and the proximal pin(s) 712 and anchor pin 716 remain in the bone segments. Advantageously, the proximal pin(s) 712 and anchor pin 716 are aligned with each other such that the rotation guide 1100 can be readily removed by sliding the rotation guide 1100 over the free ends of the proximal pin(s) 712 and anchor pin 716 while these pins remain in the bone fragments.

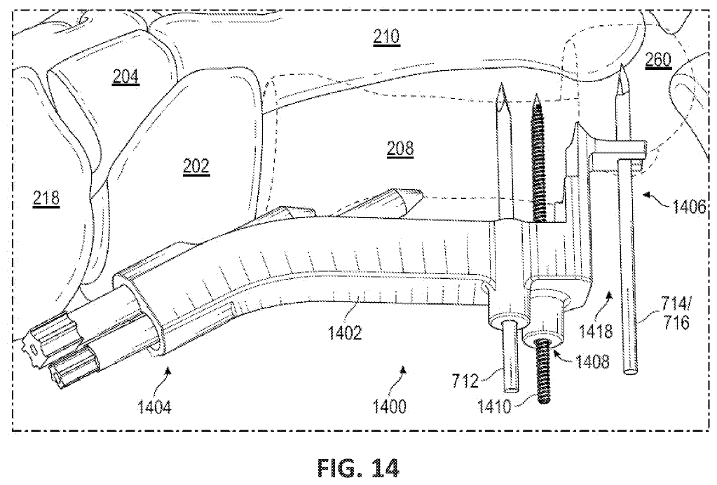

FIG. 14 is a perspective medial view of foot bones with a bone positioner 1400 deployed according to one embodiment. In certain embodiments, the bone positioner 1400 may also be referred to as an offset slide pin guide.

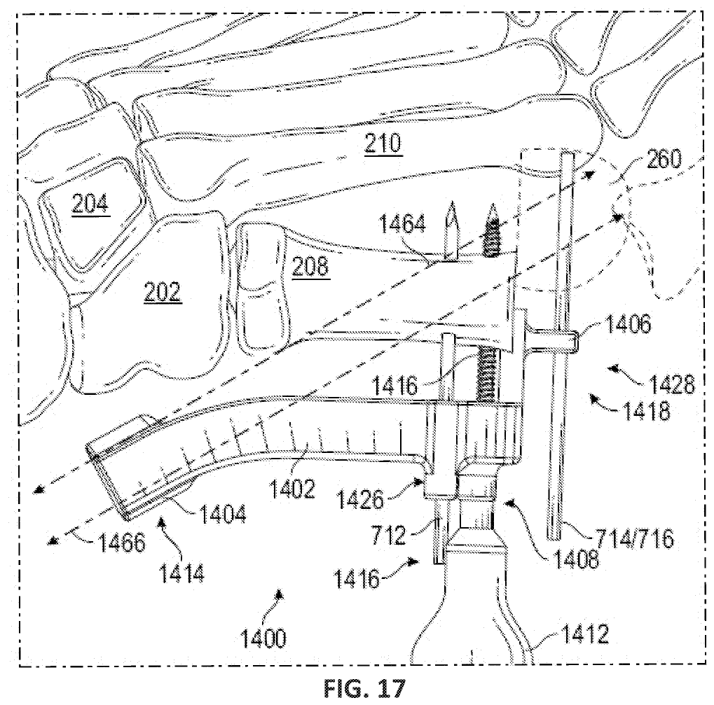

The bone positioner 1400 includes a body 1402 and has a proximal end 1404 and a distal end 1406. The bone positioner 1400 is secured to the first metatarsal 208 by one or more proximal pin(s) 712 and to the capital fragment 260 by one or more distal pin(s) 714 in FIG. 14. In certain embodiments, the anchor pin 716 may be used in place of one or more distal pin(s) 714. In FIG. 14, the bone positioner 1400 is deployed the capital fragment 260 positioned and a surgeon can proceed with a surgical procedure by deploying one or more guide pins into the first metatarsal 208. FIG. 14 also illustrates one example of a reduction guide 1408 that includes a shaft 1410 and a knob or handle 1412 (See FIG. 17).

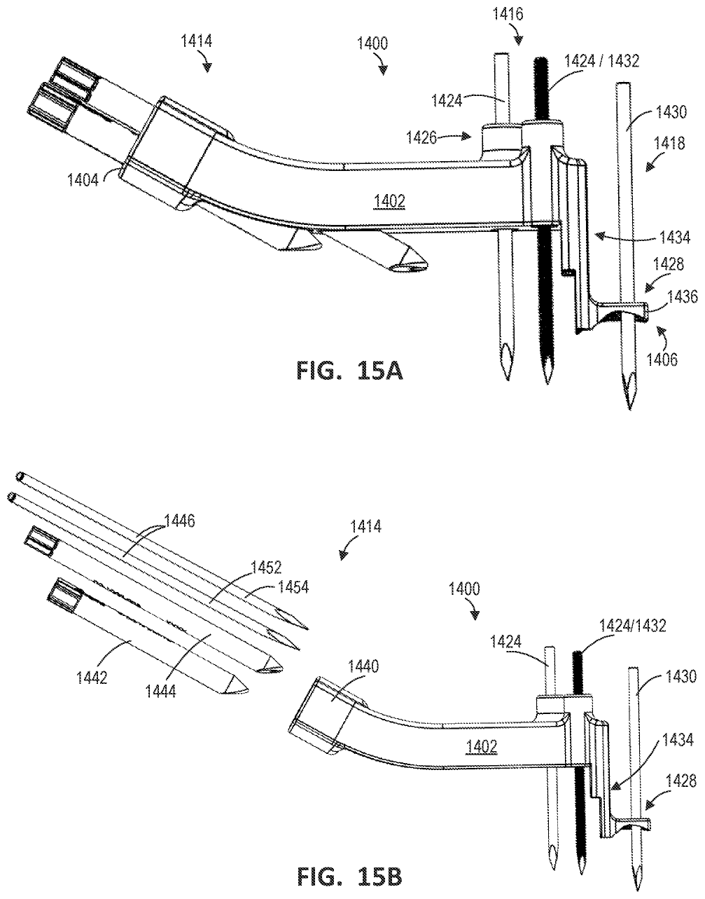

FIG. 15A is a perspective view of a bone positioner 1400 according to one embodiment. The bone positioner 1400 includes a trajectory guide 1414, a bone attachment feature 1416, and a positioning member 1418. In certain embodiments, the trajectory guide 1414 and the positioning member 1418 may, respectively, be referred to as a targeting feature and a positioning feature.

The trajectory guide 1414 provides a predetermined orientation and trajectory for deployment of fasteners, such as guide pins, into a first bone and/or a second bone or bone fragment such as a capital fragment in order to provide temporary or permanent fixation during a surgical procedure. In certain embodiments, the trajectory guide 1414 is configured to guide one or more fasteners into one or more bones at a patient-specific trajectory. A patient-specific trajectory is a trajectory that may be unique to a particular patient. In this manner, the trajectory guide 1414 may be a patient-specific trajectory guide. The guide pins may serve to guide formation of bone tunnels and/or deployment of temporary or permanent fasteners such as cannulated screws. The cannulated screws may be self-tapping and self-drilling.

Advantageously, a user or surgeon can configure the trajectory for the fasteners such that the fasteners will take an optimal angle and/or path through one or more bones, one or more spaces near bones, and/or into one or more bones. Since embodiments of the bone positioner 1400 can be fabricated based on medical imaging of a patient, the trajectory guide 1414 can be customized to the particular patient, patient needs, surgeon preferences, and the like and can also be customized to one or more types or brands of hardware fasteners and/or implants that are to be used for the surgical procedure. Alternatively, or in addition, when the bone positioner 1400 is designed and is in the form of a model, a user and/or surgeon can define, refine, adjust, and/or modify the trajectory provided by the trajectory guide 1414 such that an optimal placement of temporary and/or permanent fasteners is achieved.

The bone attachment feature 1416 secures the bone positioner 1400 to one or more bones of a patient. In the illustrated embodiment, the bone attachment feature 1416 is configured to couple the bone positioner 1400 to at least one of the first bone and the second bone. The first bone can be the first metatarsal 208 and the second bone can be a capital fragment 260. In certain embodiments, the bone attachment feature 1416 includes structure for engaging and securing bone. In other embodiments, the bone attachment feature 1416 may be combined with other features or aspects such as a reduction guide 1408 such that the bone attachment feature 1416 and reduction guide 1408 together provide a plurality of features and/or aspects.

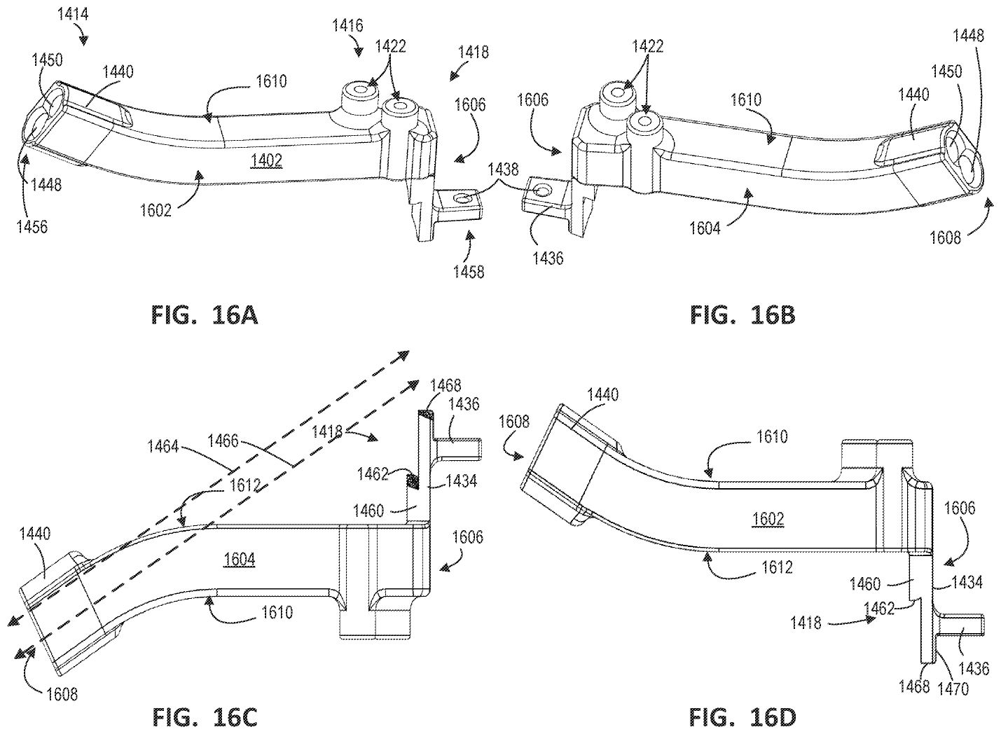

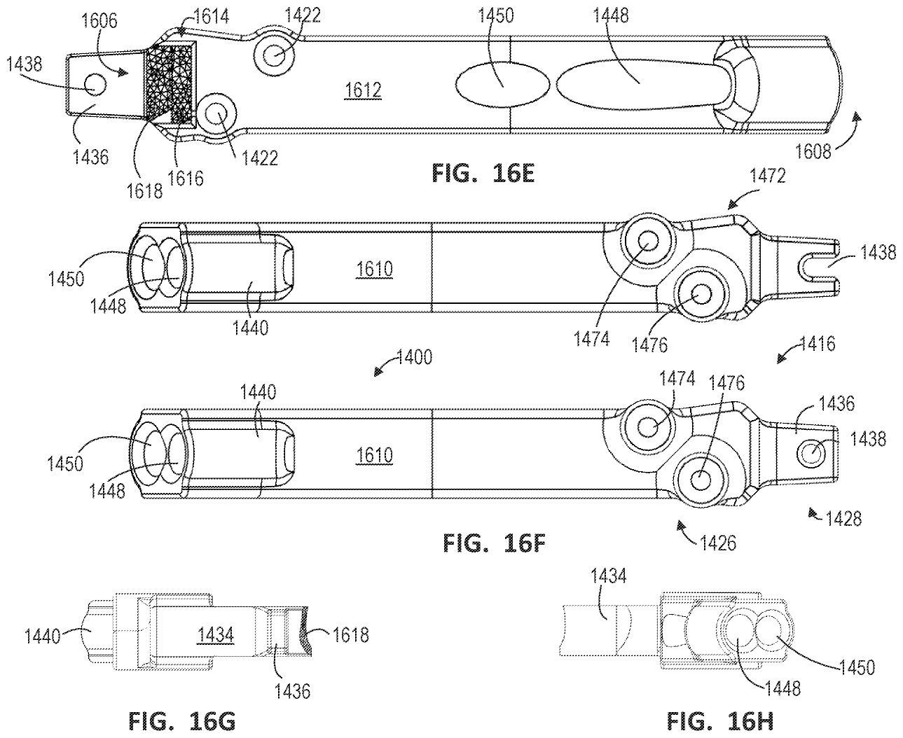

In the illustrated embodiment, the bone attachment feature 1416 includes two holes 1422 (See FIGS. 16A, 16B) that extend from one side of the bone positioner 1400 to the other. The holes can each be sized to accept one or more fasteners 1424. The one or more fasteners 1424 cooperate with the body 1402 to connect to at least a first bone. Alternatively, or in addition, the bone attachment feature 1416 includes a proximal bone attachment feature 1426 and a distal bone attachment feature 1428. The proximal bone attachment feature 1426 is configured to engage a first bone such as a first metatarsal 208 and the distal bone attachment feature 1428 is configured to engage a second bone such as a capital fragment 260. The distal bone attachment feature 1428 can include one or more fasteners 1430. In one embodiment, the fastener 1430 is the same pin, the distal pin 714, aka the anchor pin 716, used earlier in a surgical procedure.

In the illustrated embodiment, one of the one or more fasteners 1424 includes a threaded fastener 1432. The threaded fastener 1432 includes threads on the outside surface of the fastener extending from a proximal end of the threaded fastener 1432 toward a distal end. The threaded fastener 1432 may serve as part of the bone attachment feature 1416 (or proximal bone attachment feature 1426) and may, in certain embodiments, serve as a part of a reduction guide 1408, explained in more detail herein. The threaded fastener 1432 can stabilizes the bone positioner 1400 against the bone and/or skin for the procedure. The fastener 1424 and the threaded fastener 1432 can anchor the bone positioner 1400 to a bone or part of a bone such as a first metatarsal 208 or a shaft of a first metatarsal 208. Having two fasteners prevents the bone positioner 1400 from pivoting dorsally or plantarly during a procedure. In addition, a proximal bone attachment feature 1426 with two fasteners can prevent the first metatarsal 208 from twisting during a surgical procedure.

The positioning member 1418 can position a bone fragment such as a capital fragment during one or more stages of a surgical procedure. In certain embodiments, the positioning member 1418 can be combined with and/or may cooperate with the reduction guide 1408 to position and/or reduce segments of bone relative to each other.

In the illustrated embodiment, the positioning member 1418 positions and is configured to position the second bone a patient-specific distance relative to the first bone for remediating a condition present in a patient's foot. In one embodiment, the first bone is a first metatarsal 208 and the second bone is a capital fragment 260. Advantageously, the positioning member 1418 engages the two bone segments such that deployment of the bone positioner 1400 may automatically translate, rotate, and/or otherwise orient and/or position one bone relative to the other bone such that the bones are positioned to a desired position and/or orientation relative to each other for a subsequent step in a surgical procedure. In one embodiment, the subsequent step may be deployment of one or more fasteners to secure the bones in the newly positioned relationship relative to each other.

The positioning member 1418 can include a leg 1434 and a foot 1436. The leg 1434 supports and extends the foot 1436 a predetermined distance from the body 1402. In certain embodiments, the predetermined distance is patient-specific distance, unique to a particular patient, to a particular procedure, and/or to satisfy specific surgeon preferences. The patient-specific distance is a distance determined to position the capital fragment 260 for an optimal outcome of the surgical procedure. In certain embodiments, the patient-specific distance may be predetermined and provided in a preoperative plan provided to a surgeon. Alternatively, or in addition, a surgeon may be provided with a set of one or more bone positioners 1400 each with a positioning member 1418 that provided a different patient-specific distance greater than or less than an original plan or an originally recommended patient-specific distance (e.g., +10% greater, +15% greater, etc. or −10% less, −15% less, etc. and/or 10 mm greater, 15 mm greater, etc. or 10 mm less, 15 mm less, etc.).

In the illustrated embodiment, the foot 1436 may include an inferior surface that is a bone engagement surface and is contoured to engage surface of a bone fragment, such as a capital fragment, to translate and position the fragment relative to the body 1402 and/or another part of the body of a patient, such as for example a shaft of a first metatarsal 208. Alternatively, or in addition, the foot 1436 may include an inferior surface that includes a part that is a bone engagement surface and another part that engages a surface of skin of a patient medial to the capital fragment 260.

The foot 1436 may include an opening 1438 (See FIGS. 16A, 16B) that accepts a fastener 1430 that may engage the bone fragment, such as a capital fragment.

FIG. 15B illustrates more details about one embodiment of the trajectory guide 1414. The trajectory guide 1414 may include a mount 1440, one or more sleeves, and one or more fasteners, also referred to as guide pins 1446. In one embodiment, the mount 1440 supports a proximal sleeve 1442 and a distal sleeve 1444.

The mount 1440 supports and orients the one or more sleeves 1442, 1444 for deployment of the guide pins 1446 for temporary or permanent fixation within one or more bones of a patient. Advantageously, the mount 1440 includes one or more openings, such as a proximal opening 1448 and a distal opening 1450 (See FIG. 16A) that are sized and configured to accept and hold the respective sleeves 1442,1444 in a slip fit or friction fit engagement. In one embodiment, the sleeves 1442,1444 are configured to include openings 1448, 1450 and accept guide pins 1446 in which the guide pins 1446 have the same cross-sectional diameter.

In one embodiment, the cross-sectional diameter and/or length of the guide pins 1446 are different. Accordingly, the sleeves 1442,1444 may each have openings 1448, 1450 that are configured to accept guide pins 1446 of different cross-sectional diameters. For example, the proximal sleeve 1442 may be configured to include a proximal opening 1448 configured to accept a proximal guide pin 1452 having a cross-sectional diameter of approximately 1.6 millimeters. And the distal sleeve 1444 may be configured to include a distal opening 1450 configured to accept a distal guide pin 1454 having a cross-sectional diameter of approximately 1.4 millimeters.

In certain surgical procedures, deploying a guide pin transosseous (through one bone cortex on one side and out another bone cortex on another side) may require selection of a specific type, material, and/or cross-sectional diameter. This can be because a guide pin may bend or start to veer off course, off the trajectory as the distal end contacts the hard cortical surface of the bone (either the external surface and/or the internal surface). Thus, a surgeon may choose a proximal guide pin 1452 that has a larger cross-sectional diameter to mitigate the potential for veering. Similarly, the configuration of the proximal guide pin 1452 and/or the distal guide pin 1454 may be selected to support the planned permanent fasteners, e.g., cannulated screws. For example, a 1.6 millimeter diameter proximal guide pin 1452 may be suitable for deployment of a 4.0 mm diameter cannulated screw and a 1.4 millimeter diameter distal guide pin 1454 may be suitable for deployment of a 3.5 mm diameter cannulated screw. In an MIS bunion surgical procedure that may include a modified Mitchell osteotomy, the proximal fastener that is deployed using the proximal guide pin 1452 may enter the shaft of the first metatarsal 208 through the medial cortex, traverse the bone and exit through the lateral cortex traveling from a proximal medial point to a distal lateral point, the same proximal guide pin 1452 (and subsequent fastener) may then enter the capital fragment 260 through a cut face created by the osteotomy. Because the proximal fastener exits the bone and then re-enters a surgeon may desire to use a stronger fastener having a greater cross-sectional diameter. The distal fastener that is deployed using the distal guide pin 1454 may enter the shaft of the first metatarsal 208 through the medial cortex, traverse the bone and exit through a cut face of the first metatarsal 208 created by the osteotomy and may then enter the capital fragment 260 through a cut face of the capital fragment 260 created by the osteotomy. Because the distal fastener may remain within bone, the distal fastener may provide sufficient fixation with a smaller cross-sectional diameter.

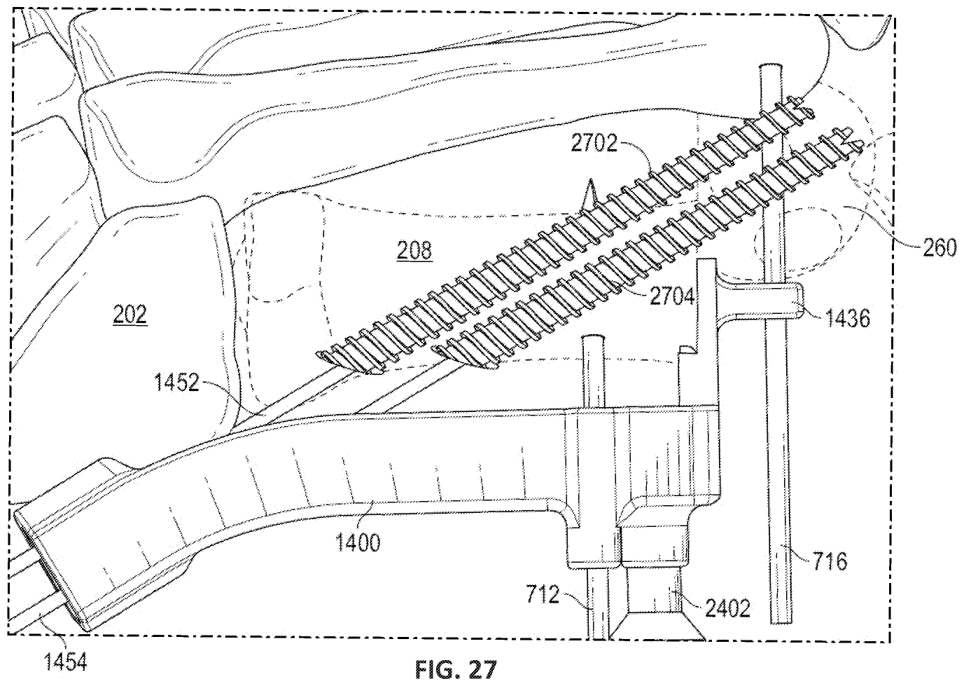

In one embodiment, the mount 1440 is configured to orient one or more sleeves 1434 for deployment of permanent fasteners, such as bone screw implants, (See FIG. 27) for a minimally invasive surgical procedure that shifts a capital fragment of a first metatarsal 208 laterally to address a bunion condition. The mount 1440 may be oriented and positioned based on imaging data based on a specific patient anatomy and/or a surgeon preference.

Advantageously, the sleeves 1442,1444 used with the mount 1440 are not permanently affixed and can be changed intraoperatively if a surgeon decides to use a different size of fasteners 1446, or permanent fasteners. A distal end of the sleeves 1442,1444 may be angled to contact a surface of a bone such as a first metatarsal 208. In certain embodiments, the sleeves 1442,1444 may include markings that clearly identify for a surgeon which opening of the mount 1440 the sleeve is to be used in. For example, in one embodiment, a proximal sleeve 1442 may be a first color and the distal sleeve 1444 may be of a different color. Furthermore, to facilitate installation of the sleeves 1442,1444 openings in the mount 1440 may be sized to accept only one of the two sleeves 1442,1444.

FIGS. 16A-16H illustrate views of a body 1402 of bone positioner 1400 according to one or more embodiments. The body 1402 includes a proximal end 1456 and a distal end 1458. The body 1402 connects the positioning member 1418 and the trajectory guide 1414. In one embodiment, the positioning member 1418 is near the distal end 1458 of the body 1402 and the trajectory guide 1414 is near the proximal end 1456 of the body 1402.

The example positioner body 1402 includes a plurality of sides including an inferior side 1602, a superior side 1604, a distal side 1606, a proximal side 1608, a medial side 1610, and a lateral side 1612. FIG. 16A is an inferior perspective view of the body 1402. FIG. 16B is a superior perspective view of the body 1402.

FIG. 16C is a view of superior side 1604 of the body 1402. FIG. 16D is a view of an inferior side 1602 of the body 1402. FIGS. 16C, 16D illustrate aspects of the positioning member 1418. The positioning member 1418 can include a base 1460, a leg 1434, and a foot 1436. The base 1460 may connect the body 1402 to the leg 1434. In the illustrated embodiment, the base 1460 is configured to engage with a first bone such as a cortex of a first metatarsal 208. In one embodiment, the base 1460 may include a bone engagement surface 1462 configured to match a contour of a cortex of a distal end of the first metatarsal 208. Alternatively, or in addition, the surface of the base may simply be curved or may be straight and flat.

The leg 1434 extends from the base 1460 towards a second bone, (e.g., a capital fragment 260). Those of skill in the art will appreciate that because the bone positioner 1400 is custom designed and/or fabricated, the length of the leg 1434 can be patient-specific and can be defined based on medical imaging data about a particular patient and/or can include a length prescribed by a surgeon for a surgical procedure.

In certain embodiments, the configuration of the positioning member 1418 can be based on a patient-specific distance a surgeon desires for the translation of the second bone, i.e., the capital fragment 260. Advantageously, the patient-specific distance that the positioning member 1418 translates the second bone can be determined based on a bone model of one or more bones of a foot of the patient. As discussed above, the bone model is generated based on medical imaging of the patient's foot. In this manner, the bone positioner 1400 is a patient-specific bone positioner.

FIG. 16C illustrates a trajectory 1464 for a proximal guide pin 1452 and a trajectory 1466 for a distal guide pin 1454. Advantageously, because the trajectory guide 1414 is designed for a specific patient and/or specific procedure, the trajectories 1464, 1466 can be predefined based on a model of one or more bones of a patient and a patient-specific trajectory 1464 and/or trajectory 1466 can be determined therefrom. In this manner, the trajectory guide 1414 is a patient-specific trajectory guide.

The leg 1434 can cooperate in defining a patient-specific distance for positioning the second bone. In the illustrated embodiment, the leg 1434 includes a lateral surface 1468 that may contact a cortex of the second bone (e.g., capital fragment 260). In the illustrated embodiment, the lateral surface 1468 is separated from the foot 1436 by an extension 1470. The extension 1470 may extend an offset distance from the foot 1436. The foot 1436 serves to hold a fastener such as anchor pin 716 in place during the surgical procedure. The length of the extension 1470, the offset distance, can be configured to account for skin pressed between a lateral surface of the foot 1436 and the lateral surface 1468 of the leg 1434 that contacts the second bone. In one embodiment, the foot 1436 can be configured to contact with the capital fragment 260 and engage with the capital fragment 260. Alternatively, or in addition, the foot 1436 may engage with the second bone (e.g., a capital fragment 260) by way of pressing against a surface of skin adjacent to the second bone (e.g., a capital fragment 260) and the skin then presses against the second bone to translate the bone as desired.

Those of skill in the art will appreciate each of the structures of the positioning member 1418 can be customized and sized to meet the needs of a particular patient and/or surgeon. In one embodiment, the length of the leg 1434, the position of the foot 1436 on the leg 1434, the thickness of the foot 1436, and/or the length of the extension 1470 can each be configured to meet the needs of a particular patient and/or surgeon because models are used to plan the surgical procedure and design the positioning member 1418. In one embodiment, the distance between the bone engagement surface 1462 and the lateral surface 1468 comprises a patient-specific lateral offset. The patient-specific lateral offset is a distance the second bone is to move laterally to assume a desired position for the surgical procedure.

FIG. 16E is a view of a lateral side 1612 of the body 1402. In the illustrated embodiment, the body 1402 includes a bone engagement surface 1614. The bone engagement surface 1614 can serve to engage at least one of a first bone and second bone. Alternatively, or in addition, the bone engagement surface 1614 can be configured to match a surface contour of at least one of a first bone and second bone. For example, the bone engagement surface 1614 may be configured to match a surface contour of a distal end of shaft of a first metatarsal 208 and to match a medial surface contour of a capital fragment 260. The bone engagement surface 1614 may be part of the bone attachment feature 1416 and/or part of the positioning member 1418.

The bone engagement surface 1614 may include a first engagement surface 1616 and a second engagement surface 1618. The first engagement surface 1616 may be patient-specific and may be configured to register to, and engage with, a bone surface of a first bone, such as a distal shaft of a first metatarsal 208. The second engagement surface 1618 may be patient-specific and may be configured to register to, and engage with, a bone surface of a second bone, such as a fragment such as a capital fragment 260. The bone engagement surface 1614 can assist a surgeon in properly positioning the bone positioner 1400 for a minimally invasive distal first metatarsal 208 bunion procedure. The lateral side 1612 may be named the lateral side 1612 because, during use, this side is directed toward a lateral side of a patient.

FIG. 16E also illustrates the proximal opening 1448 and distal opening 1450 as oval shapes because the openings 1448, 1450 may exit the body 1402 at an angle (a trajectory 1464 and trajectory 1466) and the openings 1438 are large enough to accept the sleeves 1442, 1444. The proximal opening 1448 is configured to accept and engage a proximal sleeve 1442. The distal opening 1450 is configured to accept and engage a distal sleeve 1444. Advantageously, the proximal opening 1448 and the distal opening 1450 orient proximal sleeve 1442 and the distal sleeve 1444 respectively at one or two patient-specific angles relative to a long axis of the first bone, e.g., the first metatarsal 208. As mentioned, the proximal opening 1448 and distal opening 1450 may have the same cross-sectional diameters and accept sleeves 1442, 1444 of the same diameters. Or, the proximal opening 1448 and distal opening 1450 may have different cross-sectional diameters and accept sleeves 1442, 1444 of different diameters, for example for deploying pin guides of different diameters.

FIG. 16F is a view of a medial side 1610 of a first bone positioner 1400 and a second bone positioner 1472. The second bone positioner 1472 may be another embodiment of the bone positioner 1400. In the second bone positioner 1472, the opening 1438 on the foot 1436 may be different. In the bone positioner 1400, the opening 1438 is a circular and/or cylindrical opening 1438 that extends from one surface of the foot 1436 to the other. In the second bone positioner 1472 the opening 1438 has a semi-circular cross section and is open on one side forming a shape like a horseshoe. Those of skill in the art will appreciate that the different designs for the opening 1438 may have different advantages for certain patients, surgeons, and/or surgical procedures.

In the illustrated embodiment, the opening 1438 extends from one surface of the foot to the opposite surface of the foot 1436 at a 90 degree angle, perpendicular. Alternatively, or in addition, those of skill in the art will appreciate that the opening 1438 can extend at any angle between the surfaces of the foot 1436. For example, the opening 1438 may be angled proximal to a top surface of the foot 1436, alternatively, the opening 1438 may be angled distal to a top surface of the foot 1436, alternatively, the opening 1438 may be angled plantarly to a top surface of the foot 1436, alternatively, the opening 1438 may be angled dorsally to atop surface of the foot 1436 or any angle between these. In certain embodiments, the length of the opening 1438 can be predetermined and/or can be defined such that an anchor pin 716 within the opening 1438 will maintain a stable position and/or orientation relative to the bone positioner 1400 or second bone positioner 1472.

FIG. 16F illustrates a bone positioner 1400 that includes a bone attachment feature 1416. The bone attachment feature 1416 is configured to couple the bone positioner 1400 to both a first bone and second bone. Alternatively, or in addition, the bone attachment feature 1416 may couple to just the first bone or the bone attachment feature 1416 can couple to just the second bone. In embodiments where the bone attachment feature 1416 couples to one of two bones, a user may engage the other bone for desired translation for a surgical procedure. For example, a surgeon may insert an instrument in an intramedullary canal of a first metatarsal 208 after the osteotomy to move the first metatarsal 208 relative to a capital fragment 260.

The bone attachment feature 1416 includes a proximal bone attachment feature 1426 and a distal bone attachment feature 1428. The proximal bone attachment feature 1426 is configured to engage a first bone such as a first metatarsal 208 and the distal bone attachment feature 1428 is configured to engage a second bone such as a capital fragment 260. The distal bone attachment feature 1428 can include one or more fasteners 1430.

FIG. 16F illustrates that the proximal bone attachment feature 1426 includes two openings or holes, a proximal or first opening 1474 and a distal or second opening 1476. The proximal opening 1474 is configured to accept a fastener that engages or can be deployed to engage a first bone, such as a first metatarsal 208. In one embodiment, the distal opening 1476 is configured to accept a fastener that also engages or can be deployed to also engage the first bone.

In another embodiment, the distal opening 1476 or the proximal opening 1474 can be configured to accept a reduction guide 1408. The reduction guide 1408 may be configured to draw at least one of the first bone (e.g., first metatarsal 208) and the bone positioner 1400 toward each other when activated in one manner and extend at least one of the first bone (e.g., first metatarsal 208) and the bone positioner 1400 away from each other when activated in another manner. Examples of a reduction guide 1408 that can be used with the distal opening 1476 or proximal opening 1474 and/or the distal bone attachment feature 1428 are discussed herein.

In the illustrated embodiment, the proximal opening 1474 and distal opening 1476 extend through the body 1402 from the medial side 1610 to the lateral side 1612 and perpendicular to each side. Those of skill in the art will appreciate that the openings 1474, 1476 can extend through the body 1402 at any angle needed for a procedure or to accommodate anatomy of a patient. In the illustrated embodiment, the proximal opening 1474 and distal opening 1476 are positioned in an offset configuration along the longitudinal axis of body 1402. This positioning is advantageous because with the proximal opening 1474 and distal opening 1476 in the positions illustrated fasteners that pass though the proximal opening 1474 and/or distal opening 1476 do not interfere with guide pins 1446 that will be or are deployed through the bone(s) as part of the procedure.

FIG. 16G is a view of a distal side 1606 of a bone positioner 1400. FIG. 16G illustrates a leg 1434, the second engagement surface 1618, and the mount 1440. FIG. 16H is a view of a proximal side 1608 of a body 1402. FIG. 16H illustrates openings 1438 and a leg 1434 of the body 1402.

FIG. 17 is a dorsal perspective view of a positioner 1400 according to one embodiment. The bone positioner 1400 is secured to a first metatarsal 208 and a capital fragment 260 separated from the first metatarsal 208 by an osteotomy. The proximal bone attachment feature 1426 engages the first metatarsal 208 and the distal bone attachment feature 1428 engages the capital fragment 260. In the illustrated embodiment, the proximal bone attachment feature 1426 includes a proximal pin 712 and a shaft 1410 that extend through the body 1402. The distal bone attachment feature 1428 engages the capital fragment 260 by way of a distal pin 714 or anchor pin 716 that extends through the foot 1436.

In certain embodiments, the bone attachment feature 1416 may include a reduction feature in the form of a reduction guide 1408. In another embodiment, the bone positioner 1400 may include a separate reduction guide 1408. In the illustrated embodiment, a reduction guide 1408 may be integrated into the bone attachment feature 1416. For example, the proximal bone attachment feature 1426 may include a fastener that serves as part of the proximal bone attachment feature 1426 and serves as a shaft 1410 for a reduction guide 1408.

In the illustrated embodiment, the reduction guide 1408 may include the shaft 1410 that engages with the first metatarsal 208 and extends through the body 1402 and away from the medial side 1610. In the illustrated embodiment, the shaft 1410 includes external threads along an external surface of the shaft 1410. The external threads may be configured to engage with a first bone such as the first metatarsal 208. In such an embodiment, the shaft 1410 may be deployed into the first metatarsal 208 by rotating the shaft 1410 in a direction that leverages the threads to advance the shaft 1410 into the first metatarsal 208.

Alternatively, or in addition, the shaft 1410 may include no external threads along the part of the surface of that engages with the first metatarsal 208. Instead, a conventional surface of a pin/fastener such as a K-wire may engage with the first metatarsal 208 and retain the shaft 1410 in place. In such an alternative embodiment, the shaft 1410 may include external threads along a length that is configured to engage with the knob or handle 1412.

With the shaft 1410 (and at least a threaded portion of the shaft 1410) extending away from the bone positioner 1400, the reduction guide 1408 may also include a knob or handle 1412 configured to engage with the shaft 1410. In one embodiment, the knob or handle 1412 includes an opening with internal threads configured to engage with the external threads of the shaft 1410. The opening may extend part way or all the way through the knob or handle 1412.

With the bone positioner 1400 in a desired position engaging both the first metatarsal 208 and the capital fragment 260 and at least the shaft 1410 deployed into the first metatarsal 208, a user may engage the knob or handle 1412 at a proximal end of the shaft 1410 and advance the knob along the shaft 1410 until a distal end of the knob or handle 1412 presses against the bone positioner 1400 (e.g., traverses along the shaft 1410).

At this stage, a user can begin or complete a reduction of the first metatarsal 208 relative to the capital fragment 260. In one embodiment, a user can rotate the knob or handle 1412 in a first direction (e.g., clockwise) about a long axis to the shaft 1410 and thereby draw the first metatarsal 208 towards the bone positioner 1400 as well as cause the knob or handle 1412 to traverse the shaft 1410. Similarly, the user can rotate the knob or handle 1412 in a second direction (e.g., counter-clockwise), which may be opposite the first direction, about the long axis to the shaft 1410 and thereby extend the first metatarsal 208 away from the bone positioner 1400.

As the knob or handle 1412 draws the first metatarsal 208 towards the bone positioner 1400 and/or the bone positioner 1400 towards the first metatarsal 208, the positioning member 1418 draws closer to and can engage with a medial surface of the first metatarsal 208 (e.g., by way of the first engagement surface 1616 engaging with the medial surface of the first metatarsal 208). Similarly, the positioning member 1418 draws closer to and can engage with a medial surface of the capital fragment 260 (e.g., by way of the second engagement surface 1618 engaging with the medial surface of the first metatarsal 208). In this manner, activation of the reduction guide 1408 can cooperate with the positioning member 1418 and/or the bone attachment feature 1416 to reduce the bone fragments.

In certain embodiments, the external threads may be double threads. A double thread is a pair of external threads that are along the surface of the shaft 1410. Double threads may be advantageous because the advancing or retracting of the knob or handle 1412 can be faster than using a single thread.

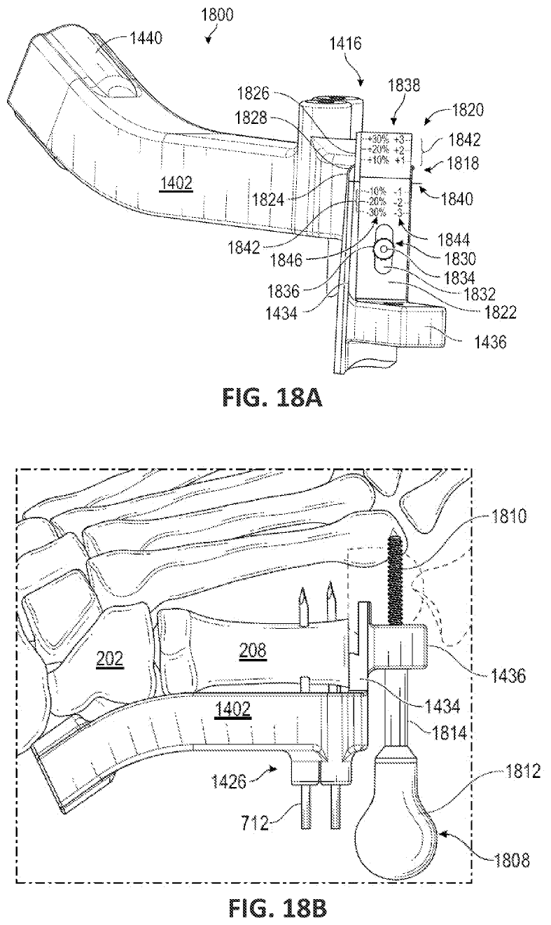

FIG. 17 also illustrates a trajectory 1464 and a trajectory 1466 that indicate the path for the guide pins 1446 through the first metatarsal 208 and into the capital fragment 260. Those of skill in the art will appreciate that the bone positioner 1400 of FIG. 17 can be a patient-specific bone positioner 1400 having each of its components specifically designed and/or configured to meet the needs of a specific patient and/or surgeon. Thus the attributes, features, and aspects of each of the components of the bone positioner 1400 and/or a system that includes the bone positioner 1400 can be predetermined and planned with a goal of achieving an optimal outcome. Such an instrument or preoperative plan may be suitable for a majority of surgical procedures. And such instruments and/or preoperative plans may be referred to as instruments or preoperative plans that are “to plan.” However, in certain cases, a surgeon may desire intraoperative flexibility and/or feature that enable certain aspects an instrument or system to be changed intraoperatively. FIG. 18A illustrate an example of such an embodiment.

FIG. 18A is an anterior view of a bone positioner 1800 according to one embodiment. The bone positioner 1800 may have many structures, features, and functions, operations, and configuration similar or identical to those of the bone positioner 1400 described earlier, like parts are identified with the same reference numerals. However, the bone positioner 1800 may include a positioning member 1818 that differs from the positioning member 1418 of the bone positioner 1400.

One difference may be that where the positioning member 1418 of the bone positioner 1400 is configured to translate a second bone relative the first bone a predetermined or patient-specific distance, the positioning member 1818 may be configured to enable a surgeon to change the translation distance intraoperatively. The positioning member 1818 may initially be configured to translate the bones a predetermined or patient-specific distance, but using an offset adjustment member 1820 of the positioning member 1818 a surgeon can increase and/or decrease the translation distance.

The offset adjustment member 1820 is any structure, system, device and/or component that enables a user to adjust a translation distance between two bones and/or bone fragments from an initial position or distance to a subsequent distance. In the illustrated embodiment, the offset adjustment member 1820 may be integrated into a positioning member 1818. The offset adjustment member 1820 may include a sliding member 1822 and a base 1824. The sliding member 1822 may connect to a leg 1434 that includes a foot 1436 similar to the bone positioner 1400. The sliding member 1822 is configured to slide with respect to the base 1824. For example, in one embodiment, the sliding member 1822 may include one or more rails 1826 and the base 1824 may include one or more the channels 1828. The one or more rails 1826 and one or more channels 1828 are configured to engage with each other and permit the sliding member 1822 to move, when in use, laterally towards the bones and medially away from the bones.

Additionally, the offset adjustment member 1820 may include a fixation member 1830 configured to fix a position of the sliding member 1822 relative to the base 1824. In the illustrated embodiment, the fixation member 1830 may take the form of a slot 1832 in the sliding member 1822, a receiving opening 1834 having internal threads, and set screw 1836 having external threads and configured to screw into the receiving opening 1834.

In the illustrated embodiment, the offset adjustment member 1820 includes at one or more markings 1838. The one or more markings 1838 communicate to a surgeon or other user how much offset, or translation distance the positioning member 1818 is providing. In the illustrated embodiment, the markings 1838 includes a baseline marking 1840 and one or more sets of offset indicators 1842, such as unit of measure indicators 1844 and/or percentage indicators 1846.

The baseline marking 1840 may include a bar on the sliding member 1822 and one or more bars on the base 1824. When the bar on the sliding member 1822 aligns with the one or more bars on the base 1824 a surgeon or user knows the offset provided by the offset adjustment member 1820 is the same as the predetermined or patient-specific distance, the offset is “to plan.” Accordingly, when the bar on the sliding member 1822 aligns with the one or more marks of the unit of measure indicators 1844 and/or the percentage indicators 1846, the surgeon or user knows the offset provided by the offset adjustment member 1820 is the amount (+1 mm, +2 mm, or −1 mm, −2 mm) or the percentage (+10%, +20% or −10%, −20%) indicated greater than or less than the predetermined or patient-specific distance, the offset is adjusted relative to “plan.”

The set screw 1836 may be installed in the receiving opening 1834 and be loose enough to permit the sliding member 1822 to slide within the channels 1828. Advantageously, a user can then manually move the sliding member 1822 within the one or more channels 1828 until a desired offset or translation is achieved, the offset or translation may the same as the plan or may be greater or may be less than the plan. Next, the surgeon or user may tighten the set screw 1836 to fix the sliding member 1822 in place to achieve the desired translation of the two bones when the bone positioner 1800 is used. In this manner, a user can operate the offset adjustment member 1820 either preoperatively or intraoperatively to potentially change the amount of translation, the offset to accomplish a very precise offset and/or a patient-specific distance between a first bone, such as a first metatarsal 208 and a second bone, such as a capital fragment 260 from a first position to a second position for remediating a condition in a patient's foot.

FIG. 18B is an anterior view of a bone positioner 1802 according to one embodiment. The bone positioner 1802 may have many structures, features, and functions, operations, and configuration similar or identical to those of the bone positioner 1400 and/or bone positioner 1800 described earlier, like parts are identified with the same reference numerals. However, the bone positioner 1802 may include a reduction guide 1808 that differs from the reduction guide 1408 of the bone positioner 1400.

In the example bone positioner 1802, the reduction guide 1808 can be implemented using an anchor pin 716 or distal pin 714 rather than a shaft 1410. Instead of a shaft 1410, the bone positioner 1802 may include a proximal pin 712 in both openings of a proximal bone attachment feature 1426. In the illustrated embodiment, the distal pin 714 or anchor pin 716 may be a threaded shaft 1810, similar to the shaft 1410. In one embodiment, the threaded shaft 1810 may include each of the features and/or aspects of the shaft 1410 including alternative embodiments.

The reduction guide 1808 may include a knob or handle 1812. The knob or handle 1812 may be a knob or handle 1812, similar to the knob or handle 1412. In one embodiment, the knob or handle 1812 may include each of the features and/or aspects of the knob or handle 1412 including alternative embodiments. In the illustrated embodiment, the knob or handle 1812 may engage with the threaded shaft 1810 and include an opening that extends into and/or through the knob or handle 1812. In certain embodiments, the knob or handle 1812 may include a neck 1814 that may be longer than a corresponding neck of the knob or handle 1412. The longer neck 1814 may enable the knob or handle 1812 to contact the foot 1436 while extending a wider part of the knob or handle 1812 further away from the body 1402 to facilitate rotating the knob or handle 1812. Those of skill in the art will appreciate that the reduction guide 1808 may operate and function in a similar way to the reduction guide 1408.

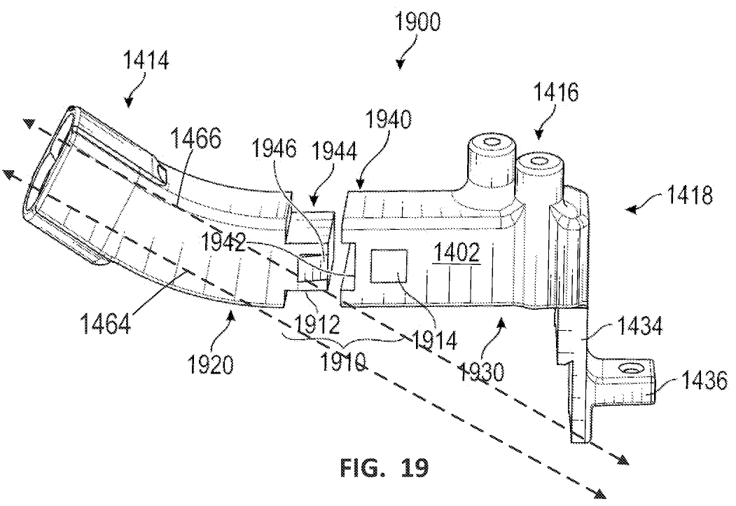

FIG. 19 is a side view of a bone positioner 1900 according to one embodiment. The bone positioner 1900 may have many structures, features, and functions, operations, and configuration similar or identical to those of the bone positioner 1400 and/or bone positioner 1800 and/or bone positioner 1802 described earlier, like parts are identified with the same reference numerals. However, the bone positioner 1900 may include a coupler 1910 that the other example bone positioner may not include.

A coupler 1910 enables the bone positioner 1900 to be made up of two or more parts. In the illustrated embodiment, the bone positioner 1900 includes a proximal part 1920 and a distal part 1930. The coupler 1910 enables the proximal part 1920 to be joined with the distal part 1930 for use during a surgical procedure, either preoperatively or intraoperatively.

In certain embodiments, having a bone positioner 1900 that includes two or more parts (e.g., proximal part 1920 and/or distal part 1930) enables one or more of the parts to be standardized and/or generic and one or more other parts to be patient-specific. For example, in the example embodiment of FIG. 19, the distal part 1930 may be standardized and the proximal part 1920 may be patient-specific.

The distal part 1930 may be standardized because a majority of patients may have a very similar shape and/or anatomical configuration for their first metatarsal 208. Thus, the positioning and offset of the capital fragment 260 in relation to the first metatarsal 208 may be standardized. Alternatively, or in addition, the distal part 1930 may include a positioning member 1818 that includes an offset adjustment member 1820 such that the amount of offset or translation can be adjusted intraoperatively. Consequently, the distal part 1930 may be reusable or may be part of a kit that includes a plurality of distal parts 1930 each with a different amount of translation for the capital fragment 260. Making the distal part 1930 reusable may reduce the overall cost for the bone positioner 1900, a system that includes the bone positioner 1900, and/or a surgical procedure.

In certain embodiments, the proximal part 1920 may be patient-specific in that a surgeon may have very specific requirements for the angle for the trajectories 1464, 1466 due to a patient's anatomy and/or treatment plan. Accordingly, the proximal part 1920 can be designed and fabricated according to the present disclosure to be a patient-specific guide, a patient-specific trajectory guide 1414. Those of skill in the art will appreciate that the proximal part 1920 may include one or more patient-specific aspects including but not limited to the trajectories 1464, 1466, a size of the proximal opening 1448, a size of the distal opening 1450, a size for the proximal guide pin 1452, a size for the distal guide pin 1454, or the like.

In one embodiment, the proximal part 1920 may be referred to as a trajectory guide because the features of a trajectory guide may be the features of the proximal part 1920. The distal part 1930 may be referred to as a bone positioner because the features of a bone positioner may be the features of the distal part 1930. Of course, the proximal part 1920 and/or distal part 1930 may go by other names. For example, the distal part 1930 may be referred to as a reduction guide. In certain embodiments, the a coupler 1910 is configured to join the bone positioner (e.g., the distal part 1930) and the trajectory guide (e.g., proximal part 1920). In such an embodiment, the bone positioner may be reusable in a subsequent surgical procedure and the trajectory guide may be a single use patient-specific apparatus.

Those of skill in the art will also appreciate that various embodiments of a coupler 1910 can be used to join the proximal part 1920 and the distal part 1930. Certain couplers 1910 may include one or more fasteners. Other couplers 1910 may include parts that snap, insert, or otherwise engage each other to join the proximal part 1920 and the distal part 1930.

In the illustrated embodiment, the coupler 1910 is implemented by way of one or more tabs 1912 that engage with one or more openings 1914. In one embodiment, a proximal end 1940 of the distal part 1930 may have an opening 1942 sized to accept a distal end 1944 of the proximal part 1920.

In one embodiment, the distal end 1944 may slide into the opening 1942. As the distal end 1944 slides into the opening 1942 the one or more tabs 1912 may slide into the one or more openings 1914. In the illustrated embodiment, each of the tabs 1912 may include a detent 1946 that can slide into the one or more openings 1914 and is biased to extend out from the one or more openings 1914.

The coupler 1910 serves to securely connect or join the proximal part 1920 to the distal part 1930. To couple the two parts, a user may slide the distal end 1944 of the proximal part 1920 into the opening 1942 until the one or more tabs 1912 position the one or more detents 1946 within the openings 1914. Once properly positioned, the one or more detents 1946 may spring, snap, click, or otherwise extend into the openings 1914. Advantageously, a user such as a surgeon may hear an auditory signal, a snap or click sound, and/or may feel a tactical signal that indicates when the coupler 1910 is properly engages and the two parts are properly coupled.

To separate the proximal part 1920 and the distal part 1930, a user can simply depress the detents 1946 within the one or more openings 1914 towards a center longitudinal axis of the bone positioner 1900 until the detent 1946 are clear of the openings 1914 and pull the proximal part 1920 and distal part 1930 apart.

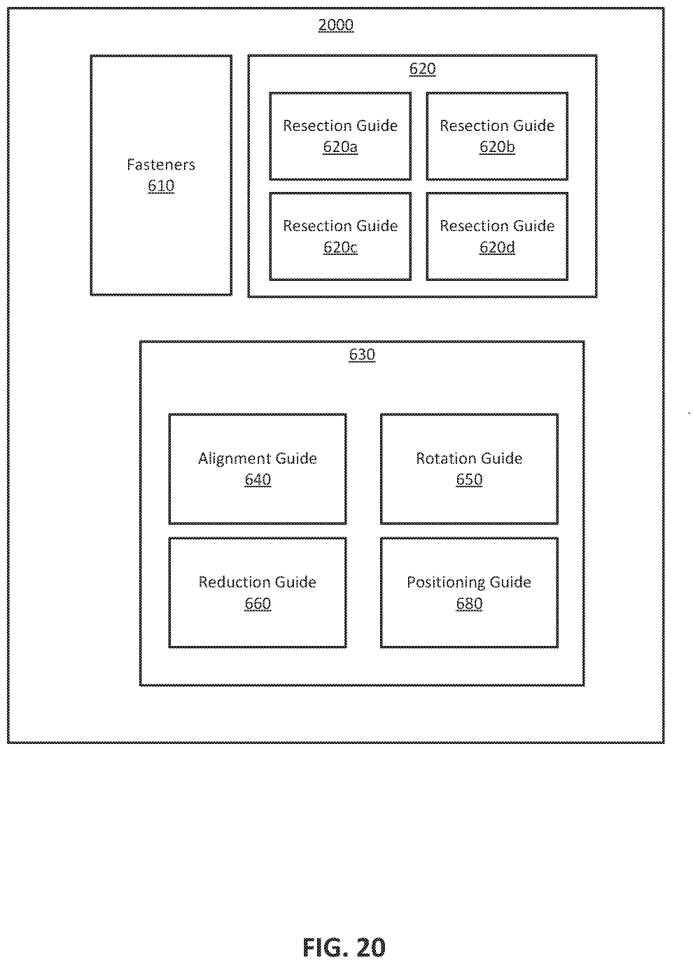

FIG. 20 illustrates an exemplary osteotomy system 2000, according to one embodiment. The osteotomy system 2000 may include one or more fasteners 610, one or more resection guides 620, one or more other complementary components 630. The osteotomy system 2000 can be used for a variety of surgical procedures.

In one embodiment, the osteotomy system 2000 may be used for a minimally invasive surgical procedure, such as a surgical procedure to correct a bunion condition. In such an embodiment, the one or more fasteners 610 can include one or more permanent fasteners and/or one or more temporary fasteners. Typically, the fasteners 610 may be used during a variety of different steps of a procedure. Temporary fasteners are often used because they can securely hold bone or parts/fragments of bones while steps of the procedure are conducted. A common temporary fastener that can be used with osteotomy system 2000 is a K-wire, also referred to as a pin or guide pin.

The osteotomy system 2000 may also include resection guide 2020 for performing an osteotomy that separates a head from a shaft of a first metatarsal 208 to form a capital fragment 260. The resection guide 2020 facilitates resection of hard tissue and/or soft tissue of a patient for a surgical procedure. In one embodiment, the resection guide 2020 can be a standalone, separate apparatus. In certain embodiments, the resection guide 2020 may be integrated with a pin guide that can be used to position a proximal pin 712 and/or a distal pin 714 in the first metatarsal 208 before an osteotomy.

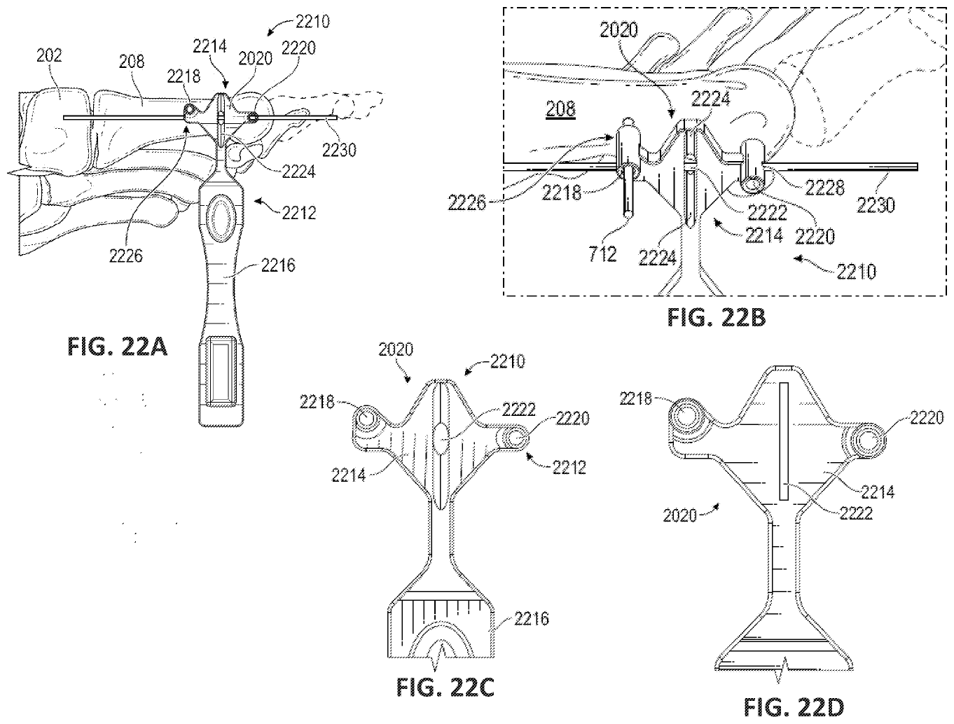

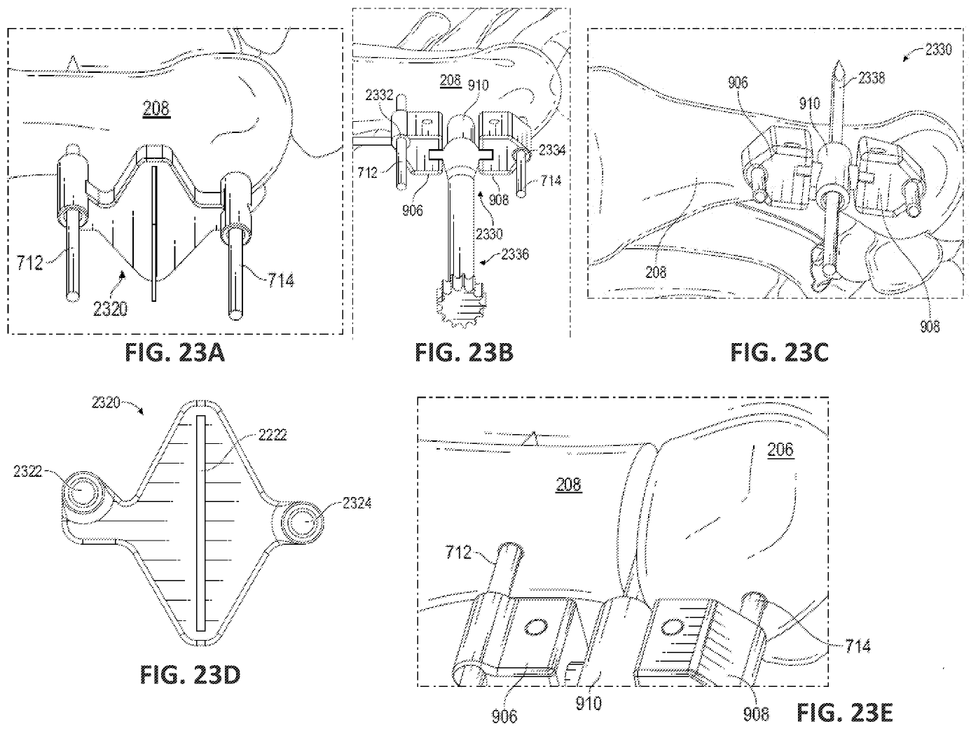

Examples of resection guide 2020 that can be used include the resection guide 700 (e.g., resection guide 620a) and/or the pivoting resection guide 902 (e.g., resection guide 620b) described herein. Other examples, of resection guide 2020 suitable for the osteotomy system 2000 include an instrument 2210 (e.g., resection guide 620c) or a resection guide 2020 (e.g., resection guide 620d) or a resection guide 2320 or a resection guide 2330 described in relation to FIGS. 22C, 22D, 23A, and 23B.

In certain embodiments, the resection guide 2020 is configured to optimize and facilitate the steps of a surgical procedure that includes the osteotomy system 2000. For example, the resection guide 2020 may include a resection guide body (e.g., body 702 or first bone attachment feature 906, second bone attachment feature 908, and cutter guide 910) that includes a proximal end that includes a proximal pin hole (e.g., proximal pin hole 708) configured to accept one of two or more pins or other fasteners and a distal end that includes a distal pin hole (e.g., distal pin hole 710) configured to accept one of two or more pins or other fasteners.

In one embodiment, the resection guide 2020 also includes a cut channel positioned to guide resection of the first metatarsal 208 for the osteotomy that forms the capital fragment 260. Where a resection guide 700 is used, the cut channel may be formed in the body 702. Where another resection guide is used, such as a pivoting resection guide 902 the cut channel may be the path a burr travels when the cutter guide 910 is used with the burr or other cutting tool to perform the osteotomy.

Those of skill in the art will appreciate that the osteotomy system 2000 can be used on humans and animals and on bones that are relatively small in comparison to other bones of the body (e.g., bones of the foot and hand). Advantageously, the osteotomy system 2000 seeks to minimize the number of fasteners or pins placed within the bones of a patient by planning a surgical procedure such that pins or fasteners placed in one stage are and/or can be reused in subsequent stages. Consequently, pins that hold the resection guide 2020 in place for the osteotomy can remain in the bone or bone fragment as instruments are deployed and/or subsequent stages of the surgical procedure are performed. For example, in one embodiment, a proximal pin 712 and distal pin 714 used for the osteotomy and with the resection guide 2020 can be reused by one or more of the complementary components 630 of the osteotomy system 2000. For example, the proximal pin 712 and/or the distal pin 714 can be reused with a positioning guide 680 such as one of the example bone positioners described herein.

The osteotomy system 2000 includes a plurality of complementary components 630. For example, the osteotomy system 2000 can include an alignment guide 640, a rotation guide 650, a reduction guide 660, and/or a positioning guide 680. Each of these complementary components 630 can be separate components or these components may be combined into one or more instruments that can be used for the surgical procedure.

In one embodiment, the alignment guide 640 can be used to align one bone with one or more other bones of the patient. In one example, a pin placement guide may include an integrated alignment guide 640 that may include holes strategically placed in the pin placement guide and a K-wire that passes through the holes to provide a visual indication of the alignment or misalignment of one bone in relation to other bones of the patient. Alternatively, or in addition, an alignment guide 640 can be used to align one or more instruments and/or implants with a desired angle, orientation, and/or trajectory. Accordingly, a trajectory guide 1414 can be one example of an alignment guide 640.

The rotation guide 650 facilitates rotational positioning of one bone fragment in relation to one or more other bones or bone fragments. In one embodiment, the rotation guide 1100 is one example of a rotation guide 650 that can be included in the osteotomy system 2000.

The reduction guide 660 facilitates reduction of one bone fragment in relation to one or more other bones or bone fragments. In one embodiment, the reduction guide 1408 is one example of a reduction guide 660 that can be included in the osteotomy system 2000. The reduction guide 1808 is another example.

The positioning guide 680 facilitates positioning of one bone fragment in relation to one or more other bones or bone fragments. In one embodiment, the positioning member 1418 is one example of a positioning guide 680 that can be included in the osteotomy system 2000. The positioning member 1818 is another example.

In certain embodiments, a single instrument may include an alignment guide 640 in the form of a trajectory guide 1414, a reduction guide 660 in the form of a reduction guide 1408, and a positioning guide 680 in the form of a positioning member 1418. In such an embodiment, a bone positioner 1400, bone positioner 1800, bone positioner 1802, and/or bone positioner 1900 can include an alignment guide 640, a reduction guide 660, and a positioning guide 680 in a single instrument.

In such an embodiment, the alignment guide 640 may include a trajectory guide 1414 that includes a proximal opening 1448 configured to accept a proximal sleeve 1442. The proximal sleeve 1442 may be configured to accept a proximal guide pin 1452 deployed into a medial cortex of a first metatarsal 208 and then into a capital fragment 260. The trajectory guide 1414 may also include a distal opening 1450 configured to accept a distal sleeve 1444. The distal sleeve 1444 may be configured to accept a distal guide pin 1454 deployed into a medial cortex of a first metatarsal 208 and then into a capital fragment 260.

In certain embodiments, the surgical procedure may call for the proximal guide pin 1452 to enter the medial cortex of the shaft of the first metatarsal 208, exit the lateral cortex of the shaft of the first metatarsal 208 and then enter the capital fragment 260 and call for the distal guide pin 1454 to enter the medial cortex of the shaft of the first metatarsal 208 and then enter the capital fragment 260, without exiting the lateral cortex of the shaft of the first metatarsal 208. In such an embodiment, the distal guide pin 1454 may be referred to as a transosseous placement feature. In other embodiments, with these requirements for the proximal guide pin 1452 the proximal guide pin 1452 may also be referred to as a transosseous placement feature.

In one embodiment, a proximal opening 1448 of the trajectory guide 1414 may extend through the trajectory guide 1414 at a first patient-specific trajectory predetermined to position a proximal fastener (e.g., a proximal guide pin 1452) within the first metatarsal 208 and the capital fragment 260. The distal opening 1450 of the trajectory guide 1414 may extend through the trajectory guide 1414 at a second patient-specific trajectory predetermined to position a distal fastener (e.g., a distal guide pin 1454) within the first metatarsal 208 and the capital fragment 260. In one embodiment, the first patient-specific trajectory may be a trajectory similar to, or the same as, the trajectory 1464. Similarly, the second patient-specific trajectory may be a trajectory similar to, or the same as, the trajectory 1466.

A bone positioner that is part of the complementary components 630 may also include a bone attachment feature, positioning member, and bone positioner body, similar in features, functions, and/or aspects to the bone attachment feature 1416, positioning member 1418, and/or body 1402 described herein. Specifically, the bone attachment feature 1416 may be configured to couple the bone positioner to the first metatarsal 208 and to the capital fragment 260 by way of two or more pins.

The positioning member 1418 is configured to translate the capital fragment 260 a patient-specific lateral offset from a position of a head of the first metatarsal 208 before an osteotomy that separates the head from the first metatarsal 208 to form the capital fragment 260. In other words, before an osteotomy that forms the capital fragment 260 the head of the first metatarsal 208 is at a distal end of the first metatarsal 208. After the osteotomy, the head is free and becomes the capital fragment 260. The patient-specific lateral offset is a patient-specific distance or amount of movement or translation that the capital fragment 260 is to make laterally relative to a new cut face of a distal end of the first metatarsal 208. In certain embodiments, this patient-specific lateral offset is determined before the surgical procedure and is based on bone models of the patient and/or a surgeon's recommendations, requests, and/or preferences. The bone positioner body is a body such as body 1402 that connects the trajectory guide 1414, bone attachment feature 1416, and positioning member 1418.

In one embodiment, a bone positioner that is part of the complementary components 630 may also include a first bone engagement surface configured to engage a medial surface of the first metatarsal. One example of the first bone engagement surface may be a first engagement surface 1616 (See FIG. 16E). The bone positioner may also include a second bone engagement surface configured to engage a medial surface of the capital fragment 260. One example of the second bone engagement surface may be a second engagement surface 1618 (See FIG. 16E).

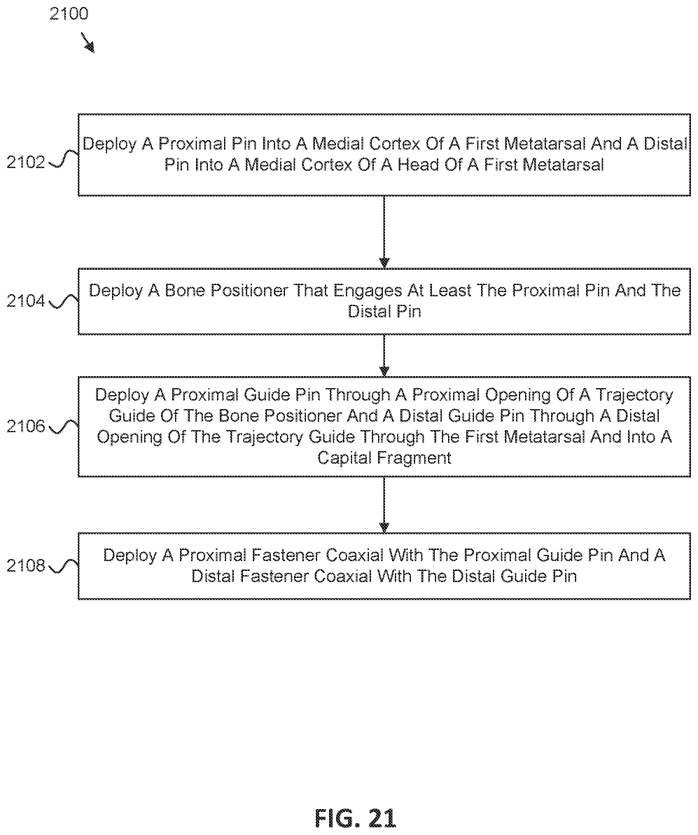

FIG. 21 is a flowchart diagram depicting a method 2100 for remediating a condition, according to one embodiment. In some implementations, one or more method steps of FIG. 21 may be performed by a device, an apparatus and/or a system.

As shown in FIG. 21, method 2100 may include deploying a proximal pin into a medial cortex of a first metatarsal and a distal pin into a medial cortex of a head of a first metatarsal (block 2102). For example, a surgeon may use an instrument 2210 (See FIG. 22) to deploy a proximal pin 712 into a medial cortex of a first metatarsal 208 and a distal pin 714 into a medial cortex of a head of a first metatarsal, as described herein. As also shown in FIG. 21, method 2100 may include deploying a bone positioner (e.g., bone positioner 1400, bone positioner 1800, bone positioner 1802, or bone positioner 1900) that engages at least the proximal pin 712 and the distal pin 714 (or an anchor pin 716 used in place of a distal pin 714) (block 2104). For example, a surgeon may deploy a bone positioner that engages at least the proximal pin 712 and the distal pin 714, as described above.

As further shown in FIG. 21, method 2100 may include deploying a proximal guide pin (e.g., proximal guide pin 1452) through a proximal opening (e.g., proximal opening 1448) of a trajectory guide (e.g., trajectory guide 1414) of the bone positioner and a distal guide pin (e.g., distal guide pin 1454) through a distal opening (e.g., distal opening 1450) of the trajectory guide (e.g., trajectory guide 1414) through the first metatarsal 208 and into a capital fragment 260 (block 2106). For example, a surgeon may deploy a proximal guide pin 1452 through a proximal opening 1448 of a trajectory guide 1414 of the bone positioner and a distal guide pin 1454 through a distal opening 1450 of the trajectory guide 1414 through the first metatarsal 208 and into a capital fragment 260, as described herein.

As also shown in FIG. 21, method 2100 may include deploying a proximal fastener (e.g., a cannulated bone screw) coaxial with the proximal guide pin (e.g., proximal guide pin 1452) and a distal fastener (e.g., a cannulated bone screw) coaxial with the distal guide pin (e.g., distal guide pin 1454) (block 2108). For example, a surgeon may deploy a proximal fastener coaxial with the proximal guide pin and a distal fastener coaxial with the distal guide pin, as described herein. In one embodiment, the surgeon may slide one or more cannulated bone screws over one or more of the proximal guide pin 1452 and/or distal guide pin 1454. Next, the surgeon may drive the one or more cannulated bone screws into the first metatarsal 208 using the proximal guide pin 1452 and distal guide pin 1454 to guide deployment.

In certain embodiments, the pin positioner may be patient-specific and may be configured to position a proximal pin 712 and a distal pin 714 and/or an anchor pin 716 in an optimal position for use by subsequent instruments. In one embodiment, the bone positioner is configured to engage with the proximal pin 712 and the distal pin 714 (and/or the anchor pin 716) that are deployed in the bone fragments. In such an embodiment, a distance between the a proximal hole that accepts the proximal pin 712 and a distal hole that accepts the distal pin 714 and/or the anchor pin 716 may be smaller than the distance used with a resection guide 620 and/or a pin positioner. The smaller distance may serve one or more of two purposes: first the smaller distance can close a gap between the first metatarsal 208 and the capital fragment 260 formed by the osteotomy and second, the smaller distance can be used to introduce compression that presses a cut face or part of the capital fragment 260 against a cut face or part of the first metatarsal 208. The compression can serve to stabilize the two bone fragments and can promote healing once the surgical procedure is completed.

Method 2100 may include additional implementations, such as any single implementation or any combination of implementations described below and/or in connection with one or more other methods or processes described elsewhere herein. In a first implementation, the process 2100 may further include deploying a pin positioner having a proximal pin hole, a distal pin hole, and a resection guide, the proximal pin hole is configured to guide the proximal pin into the first metatarsal, the distal pin hole is configured to guide the distal pin into the head of the first metatarsal; and resecting the head from the first metatarsal to form a capital fragment, by guiding a cutting tool within an opening in the resection guide.

A second or other implementation, alone or in combination with the first implementation, process 2100 may further include deploying a rotation guide (e.g., rotation guide 1100) over the proximal pin and the distal pin, the distal pin can be or is positioned within a rotation slot of the rotation guide; rotating the capital fragment relative to the first metatarsal by moving the distal pin laterally within the rotation slot to a desired position and deploying an anchor pin within an anchor hole of the rotation guide and into the capital fragment, the anchor pin replacing the distal pin (in certain embodiments, this means that the anchor pin may remain in the capital fragment 260 and the distal pin may be removed); and removing the rotation guide and leaving the proximal pin and anchor pin; and where deploying the anchor pin may include deploying a reduction guide that engages one of the first metatarsal and the capital fragment and interfaces with the bone positioner to reduce the capital fragment relative to the first metatarsal.

Although FIG. 21 shows example blocks or steps of a process 2100, in some implementations, A method 2100 may include additional steps, fewer steps, different steps, or differently arranged steps than those depicted in FIG. 21. Additionally, or alternatively, two or more of the steps of a method 2100 may be performed in parallel.

FIGS. 22A-22D illustrate different views of one or more stages in a surgical procedure that includes one or more embodiments of the present disclosure. FIG. 22 illustrates one example of a resection guide 620 and/or one or more complementary components 630 that combine functionality, features, and/or aspects of one or more instruments into a single instrument. FIG. 22 illustrates an instrument 2210 that includes functionality, features, and/or aspects of a resection guide 620, a pin positioner 2212, and an alignment guide 640.

The pin positioner 2212 facilitates placement in a bone, such as a first metatarsal 208 of a proximal pin 712 and a distal pin 714. The initial and accurate positioning of the proximal pin 712 and/or distal pin 714 can be an important step in a surgical procedure because other steps and/or instruments may rely on accurate positioning of these pins 712, 714 to perform their desired functions and aspects.

In the illustrated embodiment, the pin positioner 2212 may include a head 2214, a body 2216, proximal pin hole 2218, and a distal pin hole 2220. The head 2214 may include the proximal pin hole 2218 and the distal pin hole 2220. The head 2214 can be designed and/or fabricated based on medical imaging data of a patient's bone and bone models developed therefrom. The position and placement of the proximal pin hole 2218 and/or distal pin hole 2220 are configured to position a proximal pin 712 and a distal pin 714 respectively for subsequent steps in a surgical procedure. In the illustrated embodiment, the proximal pin hole 2218 is configured to guide the proximal pin 712 into a medial cortex of a first metatarsal 208. The distal pin hole 2220 is configured to guide the distal pin 714 into a medial cortex of a head of the first metatarsal 208.

In the illustrated embodiment, the head 2214 includes features of a resection guide 620. In particular, the head 2214 may include a slot, channel, hole, or other opening 2222 for guiding and accepting a cutting tool such as an oscillating blade and/or a burr. The size, shape, and/or configuration of the opening 2222 may depend on the kind of cutting tool that will be used. Accordingly, the type of opening 2222 included may be determined by a surgeon preference and/or equipment availability at a facility where the surgical procedure is performed.

In the illustrated embodiment, the cutting tool is a burr and the opening 2222 includes an open area that permits access to the bone. A burr can be inserted into the opening 2222 and resect the bone. Alternatively, or in addition, the opening 2222 can include a sloped carve out 2224 on two opposite sides of the part of the opening 2222 that passes through the head 2214. The two sloped carve outs 2224 may enable a surgeon to pivot a burr within the opening to resect in both a plantar and a dorsal direction. For example, when the burr is moved within a plantar sloped carve out 2224 the burr cuts in a dorsal direction and when the burr is moved within a dorsal sloped carve out 2224 the burr cuts in a plantar direction.

The body 2216 supports and connects to the head 2214. In the illustrated embodiment, the body 2216 can serve as a handle for use by a surgeon in initial positioning and placement of the instrument 2210.

Alternatively, or in addition, the instrument 2210 can includes an alignment guide 640 implemented by way of an alignment guide 2226. The alignment guide 2226 can include a plurality of holes 2228 (See FIG. 22B) and one or more alignment pins 2230. The holes 2228 can be strategically placed to pass through one or more parts of the instrument 2210 such that one or more alignment pin 2230 that are passed through the holes 2228 can serve as a visual guide for the orientation and/or direction of a long axis of a long bone such as the first metatarsal 208. In FIG. 22A an alignment pin 2230 has been passed through one or more holes 2228 of the head 2214. The alignment pin 2230 can be a K-wire. The alignment pin 2230 provides a reference for the surgeon to check visually or using a medical imaging technology such as fluoroscopy. With the alignment pin 2230 positioned and the instrument 2210 positioned a surgeon can quickly see whether the instrument 2210 is in the desired position.

In one embodiment, FIG. 22A illustrates the pin positioner 2212 as method step 2102 is about to be done. In the illustrated embodiment, the user is using the alignment guide 2226 to confirm that a distal pin hole 2220 is aligned with a long axis of the first metatarsal 208. Alternatively, or in addition, the user may use the alignment guide 2226 to confirm that the proximal pin hole 2218 is aligned with the long axis of the first metatarsal 208. In the illustrated embodiment, a horizontal alignment pin 2230 can be left in place as a proximal pin 712 is deployed into the proximal pin hole 2218. Alternatively, or in addition, a user may use flouroscopy to check the position of the pin positioner 2212 and its holes and the alignment pin 2230 relative to the parts of the bone. In certain embodiments, a surgeon may seek to position the pin positioner 2212 such that the distal pin 714 enters approximately a center of the head and proximal pin 712 enters a medial cortex of the first metatarsal 208.

FIG. 22B illustrates a stage in the surgical procedure in which the proximal pin 712 is deployed and the alignment pin 2230 is about to be removed. After removing the alignment pin 2230 the distal pin 714 can be deployed into the distal pin hole 2220.

As described above, the instrument 2210 includes a resection guide 2020 that includes the head 2214 and an opening 2222. FIGS. 22C and 22D illustrate two alternative embodiments of a resection guide 2020 that can be integrated into the instrument 2210. In FIG. 22C, the resection guide 2020 is configured to accept a burr or drill bit into the opening 2222 and includes two sloped carve outs 2224 that enable a surgeon to move the burr in a plantar and/or dorsal direction to perform the osteotomy. In FIG. 22D, the resection guide 2020 is configured to accept an oscillating saw blade (which may be narrower than a burr or drill bit) into the opening 2222 and may include a longer opening 2222 to facilitating cutting the first metatarsal 208 using the resection guide 2020 to perform the osteotomy.

FIGS. 23A and 23B illustrate two alternative embodiments of a resection guide 2020 that can be used for the method 2100 that are not integrated into an instrument such as the instrument 2210. FIG. 23A illustrates a resection guide 2320 similar to the resection guide 700 described herein connected to the first metatarsal 208. The resection guide 2320 can slide over the proximal pin 712 and distal pin 714. A surgeon may use the resection guide 2320 to perform the osteotomy of the head from the shaft of the first metatarsal 208 using an oscillating saw.

FIG. 23D illustrates a plan view of a resection guide 2320. The resection guide 2320 includes a proximal pin hole 2322 configured to slide over the proximal pin 712 and a distal pin hole 2324 configured to slide over the distal pin 714 and an opening 2222 sized to accept an oscillating saw blade.

FIG. 23B illustrates a resection guide 2330 that can be used with a burr or drill bit to do the osteotomy. The resection guide 2330 may include a proximal pin hole 2332 configured to slide over the proximal pin 712 and a distal pin hole 2334 configured to slide over the distal pin 714. The resection guide 2330 also includes a cutter guide 910 that pivots between a first bone attachment feature 906 and a second bone attachment feature 908. The resection guide 2330 may be similar to pivoting resection guide 902 described herein. The resection guide 2330 may include a handle 2336. FIG. 23C illustrates a close up view of the resection guide 2330 coupled to the first metatarsal 208. The resection guide 2330 is shown without a handle. The cutter guide 910 enables the burr 2338 to be directed dorsally and pivot to cut the bone to perform the osteotomy.

FIG. 23E illustrates the first metatarsal 208 with the connected resection guide 2330 after the osteotomy. The cut is visible and a cut face on a distal end of the first metatarsal 208 is formed and a cut face on a proximal end of the capital fragment 260 is formed. In one embodiment, the cut face on the distal end of the first metatarsal 208 can serve as a point or plane of reference for measuring where to position the proximal pin 712 and/or the distal pin 714. In one embodiment, a corresponding cut face on a bone model of a patient's first metatarsal 208 may be used to calculate measurements for the position of the proximal pin 712 and/or the distal pin 714. Advantageously, because the surgical procedure is preplanned it is known what kind of cutting tool will be used. Similarly, it is known what the width of the gap formed by the osteotomy will be. Consequently, a user may design one or more of the instruments to account for the width of this gap and/or to provide compression when closing this gap. Depending on the cutting tool used the width of the gap may be about 1 mm to about 1.5 mm.

It should be noted that during steps of a surgical procedure illustrated in stages in FIGS. 22A, 22B, 23A, 23B, 23C, 23E, very few and very small openings have been made in the skin to gain access to the first metatarsal 208. For example, in FIGS. 22A and 22B only puncture incisions or holes made by the proximal pin 712 and/or distal pin 714 may exist in the skin of the patient. In certain embodiments, prior to coupling the resection guide 2320 and/or resection guide 2330 to the bone, a small vertical (dorsal to plantar) incision may be made in the skin to provide access for the cutting tool. Said another way many of the stages of the surgical procedure to this point, may be percutaneous.

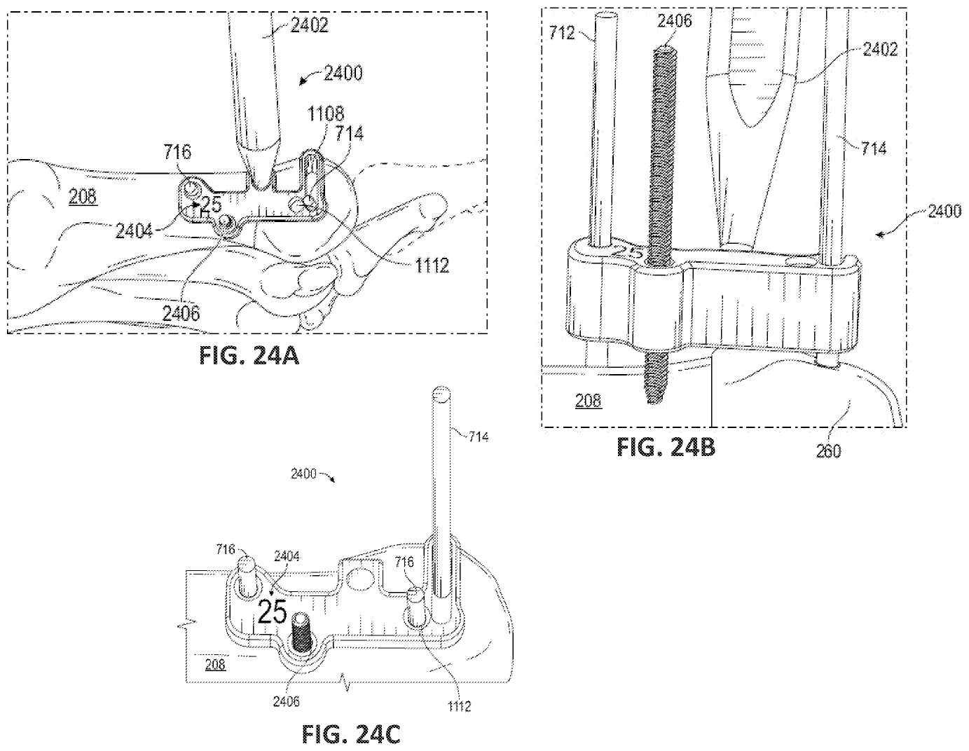

In certain embodiments, the method 2100 may also include deploying a rotation guide such as rotation guide 1100, rotating a capital fragment 260, and deploying an anchor pin 716. FIGS. 24A-24C illustrate views of a method 2100 that includes these stages. Of course, a surgeon may not use a rotation guide 1100 either because rotation is not needed or the surgeon has other ways to do the positioning and/or rotation.

FIGS. 24A-24C illustrate different views of one or more stages in a surgical procedure that includes one or more embodiments of the present disclosure. FIG. 24 illustrates an example rotation guide 2400 secured to the first metatarsal 208 using the proximal pin 712 and the distal pin 714. The rotation guide 2400 may be deployed over the proximal pin 712 and distal pin 714 used for the osteotomy. The distal pin 714 may fit within a rotation slot 1108 configured to draw the capital fragment 260 towards the first metatarsal 208 and close a gap of the osteotomy. The rotation guide 2400 may include a handle 2402. The handle 2402 may be detachable.

When the rotation guide 2400 is first deployed, the distal pin 714 may be in a plantar most position within the rotation slot 1108. A user may hold the handle 2402 and the distal pin 714 and move the distal pin 714 within the rotation slot 1108 (e.g., dorsally) to rotate the capital fragment 260 relative to the first metatarsal 208. In certain embodiments, when the distal pin 714 is moved to the dorsal most position in the rotation slot 1108 a user is assured that a predetermined amount or percentage of rotation has been done to the capital fragment 260. In one embodiment, the rotation guide 2400 may include a marking 2404 indicating how much rotation or what percentage of rotation can be provided. Of course, a user can rotate the distal pin 714 to any desired position within the rotation slot 1108 as needed.

Once a surgeon has rotated the capital fragment 260 to a desired position, the surgeon may deploy an anchor pin 716 within an anchor hole 1112 of the rotation guide 2400. Deploying the anchor pin 716 in the anchor hole 1112 secures the capital fragment 260 in a desired rotated position relative to the first metatarsal 208. At this stage, a surgeon may remove the distal pin 714. Thus, the anchor pin 716 may replace the distal pin 714. The anchor pin 716 becomes the new pin for working with the capital fragment 260 during a surgical procedure.

In certain embodiments, either before deploying the anchor pin 716 or after deploying the anchor pin 716 a user may deploy a fastener 2406. The fastener 2406 can be a shaft like shaft 1410, or another fastener 2406 that can be used with other components of an osteotomy system 2000, such as a proximal bone attachment feature 1426 and/or a reduction guide 1408. The fastener 2406 may fit within a fastener hole of the rotation guide 2400.

In one embodiment, the fastener 2406 is a threaded shaft and the fastener 2406 may be deployed by screwing the fastener 2406 into the first metatarsal 208. In one embodiment, the fastener 2406 includes threads, such as double threads along its outside surface, and the fastener 2406 may cut its own threads and drill into the bone as it is deployed. In one embodiment, the fastener 2406 is deployed as a bicortical fastener meaning the fastener 2406 may pass through both cortexes of the bone (e.g., first metatarsal 208). As described in the present disclosure, the fastener 2406 can be used for other stages such as with a bone fastener and/or with a reduction guide.

In certain embodiments, deployment of the fastener 2406 is part of deploying an anchor pin 716 and may include deploying a reduction guide that engages one of the first metatarsal 208 and the capital fragment 260. The reduction guide may interface with a bone positioner to reduce the capital fragment 260 relative to the first metatarsal 208.

In certain embodiments, an osteotomy system 2000 may include a plurality of rotation guides 2400 each configured to permit a predetermined amount or percentage of rotation for example 25% (as illustrated with marking 2404), 30%, 35%, 20%, 15%, 10%, or the like. Advantageously, a surgeon can choose which rotation guide 2400 to use based on what amount of rotation is needed either preoperatively or intraoperatively.

FIG. 24B illustrates a close up view of the rotation guide 2400 secured to the first metatarsal 208 and to the capital fragment 260 by the proximal pin 712, distal pin 714 and the fastener 2406. It should be noted that the proximal pin 712, distal pin 714 and the fastener 2406 are each parallel to each other and perpendicular to a long axis of the first metatarsal 208 and capital fragment 260. This is advantageous because with the proximal pin 712, distal pin 714 and the fastener 2406 so positioned a user can readily slide the rotation guide 2400 off of the proximal pin 712, distal pin 714 and the fastener 2406 without removing any of them. Similarly, the anchor pin 716 is configured to enter the bone parallel to the proximal pin 712 and the fastener 2406, thus with the proximal pin 712, anchor pin 716, and fastener 2406 deployed the rotation guide 2400 can be readily slid off of these fasteners/pins.

FIG. 24C illustrates a close up view of the rotation guide 2400 secured to the first metatarsal 208 and to the capital fragment 260 by the proximal pin 712, distal pin 714 and the fastener 2406. The anchor hole 1112 is shown and the handle 2402 is detached.

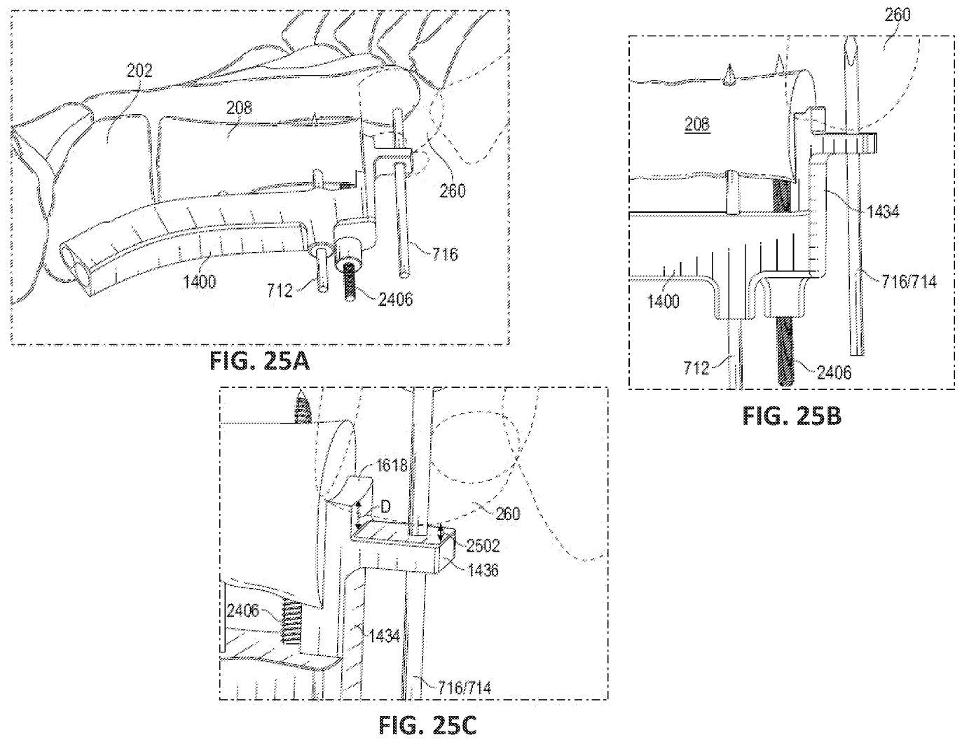

FIGS. 25A-25C illustrate different views of one or more stages in a surgical procedure that includes one or more embodiments of the present disclosure. FIG. 25A illustrates a stage of the method 2100 in which a bone positioner, such as bone positioner 1400, is deployed that engages the proximal pin 712, the anchor pin 716 and/or a fastener 2406. Those of skill in the art will appreciate that while the surgical procedure is a minimally invasive procedure and/or a percutaneous procedure, a surgeon may need to enlarge an incision or make an incision in medial skin of the patient to provide access for the bone positioner 1400 to access the first metatarsal 208 and capital fragment 260. In one embodiment, holes or openings in the bone positioner 1400 accept and slide over the proximal pin 712, fastener 2406, and/or distal pin 714.

At this stage, in FIG. 25A, the proximal pin 712 has been deployed into the medial cortex of a first metatarsal 208 and the anchor pin 716 (or a distal pin 714) has been deployed into the medial cortex of the head of the first metatarsal 208. In certain embodiments, a fastener 2406 is deployed into the first metatarsal 208. Those of skill in the art will appreciate that as the bone positioner 1400 is slid along the pins 712, 714, 716, 2406, the first engagement surface 1616 approaches the medial surface of the first metatarsal 208 and the second engagement surface 1618 approaches the medial surface of the capital fragment 260 (shown transparent). The first engagement surface 1616 and second engagement surface 1618 can be below the surface of the skin. The foot 1436 approaches the surface of the skin. As the bone positioner 1400 moves along the pins, we get to the stage illustrated in FIG. 25A, the first engagement surface 1616 contacts the medial surface of the first metatarsal 208, the second engagement surface 1618 contacts the medial surface of the capital fragment 260, and the foot 1436 contacts the surface of the skin. As the foot 1436 contacts the skin and the second engagement surface 1618 contacts the capital fragment 260 the bone positioner 1400 translates the capital fragment 260 laterally with respect to the first metatarsal 208.

Those of skill in the art will appreciate that in certain embodiments a bone positioner may not include a first engagement surface 1616 and/or a second engagement surface 1618 and instead of these surfaces contacting cortices of the bones, the foot 1436 may press against the skin adjacent to the capital fragment 260 and a lateral side 1612 may press against medial skin alongside the first metatarsal 208 to position and/or translate the capital fragment 260 relative to the first metatarsal 208. Alternatively, or in addition, the a bone positioner may include just one of a first engagement surface 1616 and/or a second engagement surface 1618 which may contact the first metatarsal 208 and/or the capital fragment 260 while a foot 1436 and/or the lateral side 1612 may contact skin of the patient to position and/or translate the capital fragment 260 relative to the first metatarsal 208.

FIG. 25B illustrates a close up view of the stage depicted in FIG. 25A. The bone positioner 1400, proximal pin 712, anchor pin 716 or distal pin 714, fastener 2406, the leg 1434, and the foot 1436 are illustrated for one embodiment. FIG. 25C illustrates a closer view that the stage depicted in FIG. 25B. FIG. 25C illustrates a gap 2502 between the foot 1436 and the capital fragment 260. A patient's skin and other soft tissue sits within this gap 2502. In certain embodiments, when the bone positioner 1400 is designed and fabricated based on medical imaging data of a patient, the size of the gap 2502 can be determined. In one embodiment, the size of the gap 2502 is considered and used to determine a patient-specific distance “D” between a lateral surface of the foot 1436 and the second engagement surface 1618.

In certain embodiments, a bone positioner 1400 may apply more pressure and/or translation force on the surface of the skin adjacent to the medial cortex of the capital fragment 260 than the second engagement surface 1618 applies to the medial cortex of the capital fragment 260. In some cases, the foot 1436 may contact and press on the skin and capital fragment 260 while the second engagement surface 1618 may not contact the medial cortex of the capital fragment 260. Advantageously, because the present disclosure uses a bone model of the patient's bones and, in certain embodiments, can include a model of the patient's skin in this part of the body, the sizes, dimensions, lengths and configurations of the positioning guide 680, positioning member 1418, and/or positioning member 1818 such as the sizes, offsets, and/or relationships between the leg 1434, foot 1436, first engagement surface 1616, second engagement surface 1618, hole that passes through the foot 1436, and/or anchor pin 716 can each be changed, adapted, revised, and/or customized to meet the needs and/or preferences of the patient and/or surgeon. For example, in one embodiment, a distance between holes for the proximal pin 712 and/or fastener 2406 and the anchor pin 716 can be smaller than the distance used in other instruments such as a resection guide 620, pin positioner 2212, or the like in order to provide compression of the capital fragment 260 towards the first metatarsal 208.

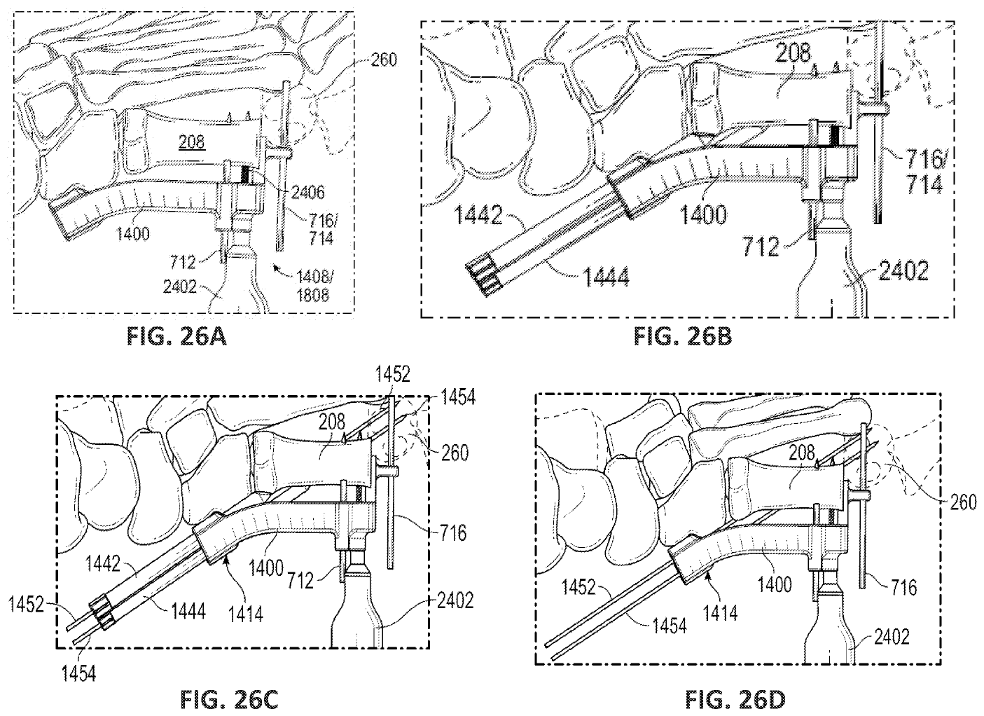

FIGS. 26A-26D illustrate different views of one or more stages in a surgical procedure that includes one or more embodiments of the present disclosure. FIG. 26A illustrates a stage of the method 2100 in which a bone positioner, such as bone positioner 1400, is deployed that engages the proximal pin 712, the anchor pin 716 and/or a fastener 2406. In addition, a handle 2402 has been screwed onto the fastener 2406. The handle 2402 and fastener 2406 may cooperate with a body 1402 of the bone positioner 1400 to form a reduction guide 1408, reduction guide 1808. A user such as a surgeon has screwed the handle 2402 down until it contacts the body 1402. The reduction guide 1408, 1808 can serve as a driver to translate the capital fragment 260 relative to the first metatarsal 208. A user may stop turning the handle 2402 when the first engagement surface 1616 contacts the surface of the first metatarsal 208. By using the bone positioner 1400, handle 2402 and fastener 2406 a user can readily reduce the first metatarsal 208 and capital fragment 260.

FIG. 26B illustrates a subsequent stage which may be part of the method 2100 in which the bone positioner 1400 is deployed and the bones are reduced and sleeves are deployed. In one embodiment, the proximal sleeve 1442 and/or distal sleeve 1444 can be deployed after the bone positioner 1400 is deployed and the bones are reduced. Alternatively, or in addition, the proximal sleeve 1442 and/or distal sleeve 1444 can be positioned within the bone positioner 1400 before the bone positioner 1400 is deployed.

FIG. 26C illustrates a subsequent stage which may be part of the method 2100 in which the bone positioner 1400 and sleeves are deployed and a user has deployed a proximal guide pin 1452 and a distal guide pin 1454. A surgeon may deploy the proximal guide pin 1452 through a proximal opening 1448 (in one embodiment, by way of the proximal sleeve 1442) of the trajectory guide 1414. A surgeon also may deploy the distal guide pin 1454 through a distal opening 1450 (in one embodiment, by way of the distal sleeve 1444) of the trajectory guide 1414.

FIG. 26C illustrates that in one embodiment, the proximal guide pin 1452 enters the first metatarsal 208 near a corner of a proximal medial cortex of the first metatarsal 208. The proximal guide pin 1452 travels through the first metatarsal 208 and exits the first metatarsal 208 near a corner of a distal lateral cortex of the first metatarsal 208. Next, the proximal guide pin 1452 passes into a proximal cut face of the capital fragment 260 and a distal end of the proximal guide pin 1452 may remain in the capital fragment 260 during this stage. In certain embodiments, the bone positioner 1400 and/or trajectory guide 1414 can be designed and fabricated to ensure that a cannulated screw (particularly a medial edge of the screw) that is deployed over the proximal guide pin 1452 exits the first metatarsal 208 and then enters the capital fragment 260.

FIG. 26C also illustrates that in one embodiment, the distal guide pin 1454 enters the first metatarsal 208 near a corner of a proximal medial cortex of the first metatarsal 208. The distal guide pin 1454 travels through the first metatarsal 208 and exits the first metatarsal 208 through a cut face formed by the osteotomy of the first metatarsal 208. Next, the distal guide pin 1454 passes into a proximal cut face of the capital fragment 260 and a distal end of the distal guide pin 1454 may remain in the capital fragment 260 during this stage. In certain embodiments, the bone positioner 1400 and/or trajectory guide 1414 can be designed and fabricated to ensure that a cannulated screw deployed over the distal guide pin 1454 remains within the first metatarsal 208 and the capital fragment 260.

In certain embodiments, the path of the proximal guide pin 1452 is referred to as in, out, in because proximal guide pin 1452 enters the first metatarsal 208, exits the first metatarsal 208, and then enters the capital fragment 260. Similarly, the path of the distal guide pin 1454 may be referred to as all in because distal guide pin 1454 enters the first metatarsal 208 and remains within the first metatarsal 208 until the distal guide pin 1454 enters the capital fragment 260. Those of skill in the art will appreciate that the path and configuration of the proximal guide pin 1452 and/or distal guide pin 1454 may be the same as that for the cannulated screws deployed using these guide pins.

FIG. 26D illustrates a subsequent stage which may be part of the method 2100 in which the bone positioner 1400 and guide pins 1452,1454 are deployed. At this stage, the first metatarsal 208 and capital fragment 260 are in a stable state and are fixed in their position and orientation. A surgeon can relax knowing the bones are reduced and stable.

FIG. 27 illustrates a stage in a surgical procedure that includes one or more embodiments of the present disclosure. FIG. 27 illustrates a stage which may be part of the method 2100 in which the guide pins 1452,1454 are deployed and a user has removed the sleeves and deployed a proximal fastener such as a bone screw 2702 and a distal fastener such as a bone screw 2704. Advantageously, the bone screw 2702 and bone screw 2704 are deployed using the proximal guide pin 1452 and the distal guide pin 1454. Thus, the proximal fastener (e.g., bone screw 2702) is deployed coaxial with the proximal guide pin 1452 and the distal fastener (e.g., bone screw 2704) is deployed coaxial with the distal guide pin 1454. In certain embodiments, a user may back out the anchor pin 716 in order to make room in the capital fragment 260 for the bone screws 2702,2704.

Advantageously, using the apparatus, systems, and/or methods of the present disclosure the surgeon may have a preoperative plan that identifies which specific bone screw (length, width, diameter, thread, pitch, etc.) to use for the proximal fastener and which specific bone screw to use for the distal fastener. In the illustrated embodiment, the bone screws 2702,2704 have a tapered proximal end so that the proximal end will be substantially flush with the medial cortex. Alternatively, or in addition, the bone screws 2702,2704 may include external threads, may be self-tapping, and may have a distal end that enables the bone screws 2702,2704 to be self-drilling. In certain embodiments, as a surgeon deploys the bone screws 2702,2704 and/or after deploying the bone screws 2702,2704 a surgeon may check the alignment, trajectory and/or depth using flouroscopy. In the illustrated embodiment, the bone screws 2702,2704 may be implants that serve as permanent fixation for the first metatarsal 208 and capital fragment 260.

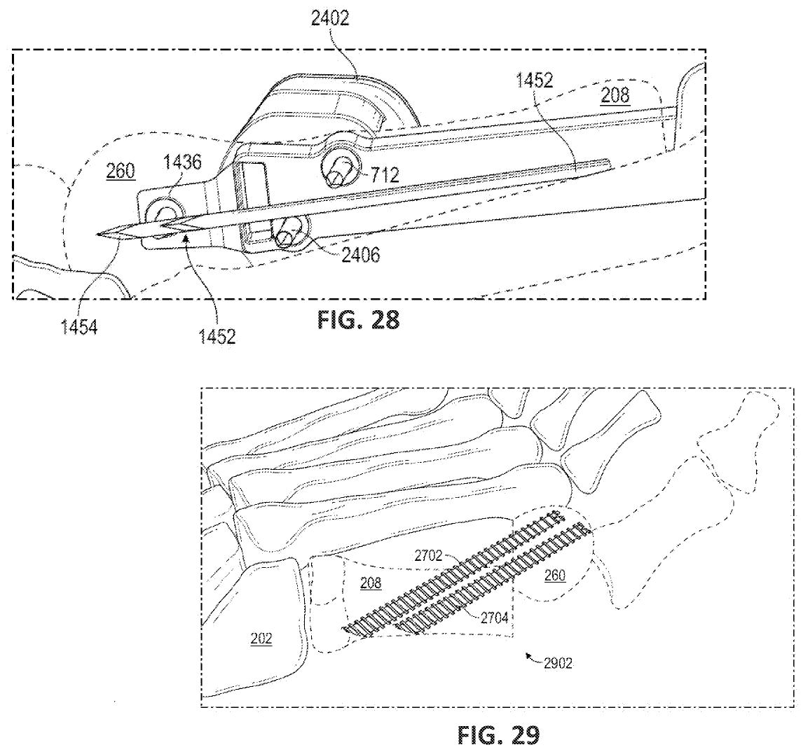

FIG. 28 illustrates a stage in a surgical procedure that includes one or more embodiments of the present disclosure. In FIG. 28, the proximal guide pin 1452 and distal guide pin 1454 have been deployed. FIG. 28 illustrates that the holes for the proximal pin 712 and fastener 2406 are specifically designed and/or positioned such that the proximal pin 712 and/or fastener 2406 within the first metatarsal 208 will not interfere with the proximal guide pin 1452 or the distal guide pin 1454 and/or not interfere with the bone screws 2702,2704 deployed in a later stage.

FIG. 29 illustrates a stage in a surgical procedure that includes one or more embodiments of the present disclosure. FIG. 29 illustrates a stage which may be part of the method 2100 in which the proximal fastener and distal fastener are deployed (e.g., bone screws 2702,2704). A user has removed the bone positioner 1400, proximal pin 712, anchor pin 716, and fastener 2406. In certain embodiments, a user may optionally then use a burr, oscillating saw, or other cutting tool (e.g., an osteotome) to shape, resect, or shave down a distal medial corner 2902 of the first metatarsal 208.

Advantageously, the present disclosure provides an apparatus, system, and/or method that can remediate a condition in a patient's foot. Those of skill in the art will appreciate that the methods, processes, apparatuses, systems, devices, and/or instruments of the present disclosure can be used to address a variety of conditions in a variety of procedures and/or parts of the body of the patient. The present disclosure can provide a patient-specific positioner and/or a plurality of positioners that each have differences in the trajectories or angles for the guide pins and/or an amount of translation provided between the capital fragment 260 and the first metatarsal 208.

Conventionally, correction methods, systems, and/or instrumentation for a condition such as a bunion and/or a hallux valgus, face several challenges. One example is how to target and position the fasteners including the proximal fastener (e.g., bone screw 2702) so that is passes into and out of the first metatarsal 208 and then into the capital fragment 260. Another example is how to keep a pin guide traversing through the first metatarsal 208 from bending as the pin guide contacts the lateral cortex of the first metatarsal 208. Another challenge is how to translate the capital fragment 260 and retain the capital fragment 260 to ensure proper healing of the bones after the procedure. Another challenge is how to rotate and manipulate the capital fragment 260 relative to the first metatarsal 208, for example to move the sesamoids to face plantarly. Advantageously, the present disclosure can address many, if not all of these challenges to assist a surgeon in performing the surgical procedure and improve the quality of patient care and outcomes.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

1. An apparatus for remediating a condition present in a patient's foot, the apparatus comprising:
a bone positioner comprising:
a bone attachment feature configured to couple the bone positioner to at least one of a first bone and a second bone; and
a positioning member configured to position the second bone a patient-specific distance relative to the first bone for remediating a condition present in a patient's foot.

2. The apparatus of claim 1, further comprising:
a trajectory guide configured to guide one or more fasteners into one or more bones at a patient-specific trajectory.

3. The apparatus of claim 2, further comprising:
a body having a proximal end and a distal end, the body connecting the positioning member and the trajectory guide; and
wherein the positioning member is near the distal end of the body and the trajectory guide is near the proximal end of the body.

4. The apparatus of claim 2, further comprising:
a coupler configured to join the bone positioner and the trajectory guide; and
wherein the bone positioner is reusable in a subsequent surgical procedure and the trajectory guide is a single use patient-specific apparatus.

5. The apparatus of claim 2, wherein the patient-specific distance and the patient-specific trajectory are determined based on a bone model of one or more bones of a patient, the bone model generated based on medical imaging of the patient's foot and wherein the bone positioner is a patient-specific bone positioner and the trajectory guide is a patient-specific trajectory guide.

6. The apparatus of claim 2, wherein the trajectory guide comprises:
a proximal opening configured to receive a proximal sleeve;
a distal opening configured to receive a distal sleeve; and
wherein one of the proximal opening and the distal opening are configured to orient the proximal sleeve and the distal sleeve at a patient-specific angle relative to a long axis of the first bone.

7. The apparatus of claim 1, wherein the positioning member further comprises:
a base that engages with the first bone;
a leg that extends from the base toward the second bone; and
a foot connected to the leg, the foot configured to engage with the second bone.

8. The apparatus of claim 1, wherein the bone positioner further comprises:
a bone engagement surface configured to match a surface contour of at least one of the first bone and the second bone.

9. The apparatus of claim 1, wherein the bone attachment feature comprises:
a proximal bone attachment feature configured to engage the first bone; and
a distal bone attachment feature configured to engage the second bone.

10. The apparatus of claim 9, wherein the proximal bone attachment feature comprises:
a first opening in the proximal bone attachment feature, the first opening configured to accept a fastener that engages the first bone; and
a second opening in the proximal bone attachment feature, the second opening configured to accept a reduction guide that engages the first bone, the reduction guide configured to draw at least one of the first bone and the bone positioner toward each other when activated in one manner and extend at least one of the first bone and the bone positioner away from each other when activated in another manner.

11. The apparatus of claim 10, wherein the reduction guide comprises:
a shaft comprising external threads along an external surface of the shaft and configured to engage the first bone;
a knob comprising an opening configured to accept the shaft, the opening comprising internal threads configured to engage the external threads of the shaft; and
wherein the knob is configured to traverse the shaft when the knob is rotated about the shaft in a first direction and draw the first bone towards the bone positioner and to traverse the shaft when the knob is rotated about the shaft in a second direction and extend the first bone away from the bone positioner.

12. The apparatus of claim 9, wherein the bone attachment feature is configured to couple the bone positioner to both the first bone and the second bone, the distal bone attachment feature comprising an opening, the opening configured to receive a fastener that engages the second bone.

13. The apparatus of claim 1, wherein the positioning member comprises:
an offset adjustment member coupled to the bone positioner and configured to change a position of the second bone relative to the first bone from an initial position to a second position for remediating a condition present in a patient's foot.

14. A system for remediating a condition present in a patient's foot, the system comprising:
a bone positioner comprising:
a trajectory guide comprising:
a proximal opening configured to accept a proximal sleeve configured to accept a proximal guide pin deployed into a medial cortex of a first metatarsal and a capital fragment;
a distal opening configured to accept a distal sleeve configured to accept a distal guide pin deployed into a medial cortex of the first metatarsal and the capital fragment;
a bone attachment feature configured to couple the bone positioner to the first metatarsal and to the capital fragment by way of two or more pins;
a positioning member configured to translate the capital fragment a patient-specific lateral offset from a position of a head of the first metatarsal before an osteotomy that separates the head from the first metatarsal to form the capital fragment; and
a bone positioner body that connects the trajectory guide, the bone attachment feature, and the positioning member; and
a resection guide comprising:
a resection guide body comprising:
a proximal end having a proximal pin hole configured to accept one of the two or more pins;
a distal end having a distal pin hole configured to accept one of the two or more pins; and
a cut channel positioned to guide resection of the first metatarsal for the osteotomy that forms the capital fragment.

15. The system of claim 14, wherein the proximal opening of the trajectory guide extends through the trajectory guide at a first patient-specific trajectory predetermined to position a proximal fastener within the first metatarsal and the capital fragment and the distal opening of the trajectory guide extends through the trajectory guide at a second patient-specific trajectory predetermined to position a distal fastener within the first metatarsal and the capital fragment and wherein the bone positioner comprises:
a first bone engagement surface configured to engage a medial surface of the first metatarsal; and
a second bone engagement surface configured to engage a medial surface of the capital fragment.

16. The system of claim 14, wherein the resection guide further comprises a pin positioner comprising a proximal pin hole configured to guide a proximal pin into a medial cortex of the first metatarsal and a distal pin hole configured to guide a distal pin into a medial cortex of a head of the first metatarsal.

17. The system of claim 14, further comprising:
a rotation guide comprising:
a rotation guide body having at least two pin holes configured to engage the two or more pins, the rotation guide body comprising:
a proximal end;
a distal end comprising a rotation slot configured to enable rotation of the capital fragment relative to the first metatarsal; and
an anchor hole configured to receive an anchor pin deployed in the capital fragment having a desired rotated position relative to the first metatarsal.

18. A method for remediating a condition present in a patient's foot, the method comprising:
deploying a proximal pin into a medial cortex of a first metatarsal and a distal pin into a medial cortex of a head of a first metatarsal;
deploying a bone positioner that engages at least the proximal pin and the distal pin;
deploying a proximal guide pin through a proximal opening of a trajectory guide of the bone positioner and a distal guide pin through a distal opening of the trajectory guide through the first metatarsal and into a capital fragment; and
deploying a proximal fastener coaxial with the proximal guide pin and a distal fastener coaxial with the distal guide pin.

19. The method of claim 18, further comprising:
deploying a pin positioner comprising a proximal pin hole, a distal pin hole, and a resection guide, the proximal pin hole configured to guide the proximal pin into the first metatarsal, the distal pin hole configured to guide the distal pin into the head of the first metatarsal; and
resecting the head from the first metatarsal to form a capital fragment, by guiding a cutting tool within an opening in the resection guide.

20. The method of claim 18, further comprising:
deploying a rotation guide over the proximal pin and the distal pin, the distal pin positioned within a rotation slot of the rotation guide;
rotating the capital fragment relative to the first metatarsal by moving the distal pin laterally within the rotation slot to a desired position and deploying an anchor pin within an anchor hole of the rotation guide and into the capital fragment, the anchor pin replacing the distal pin;
removing the rotation guide and leaving the proximal pin and anchor pin; and
wherein deploying the anchor pin further comprising deploying a reduction guide that engages one of the first metatarsal and the capital fragment and interfaces with the bone positioner to reduce the capital fragment relative to the first metatarsal.
